(19) United States
(12) Patent Application Publication
(10) Pub. No.: US 2026/0049101 A1
(43) Pub. Date: Feb. 19, 2026

(54) TOPICAL TREATMENT OF SKIN CANCER USING OLIGOPEPTIDES

(71) Applicant: BACOBA AG, Buonas (CH)

(72) Inventors: Michael ADAMS, Uster (CH); Michael KEMMLER, Lörrach (DE); Dimanthi PLIATSIKA, Wädenswil (CH); Rainer RIEDL, Wilen b. Wollerau (CH)

(21) Appl. No.: 19/100,968

(22) PCT Filed: Aug. 9, 2023
(86) PCT No.: PCT/EP2023/072062

§ 371 Date: Feb. 4, 2025

(30) Foreign Application Priority Data
Aug. 10, 2022 (EP) 22189654.1

(51) Int. Cl. C07K 7/06; A61K 38/00; A61P 35/00

(52) U.S. Cl. CPC C07K 7/06; A61P 35/00; A61K 38/00

(57) ABSTRACT

The present invention relates to a compound of formula (I), pharmaceutically acceptable salts thereof as well as pharmaceutical compositions comprising the same and, in particular, the compounds of formula (I) and pharmaceutical compositions for use in a method of topical treatment of a skin cancer of a mammal, wherein said method comprises topical administration of said compound to said mammal.

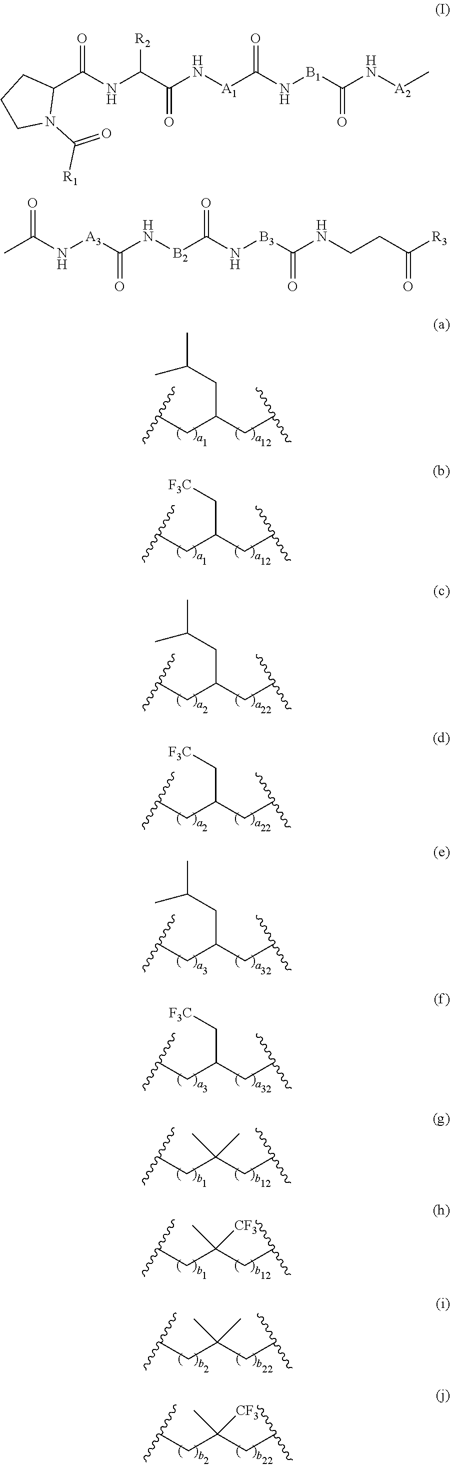

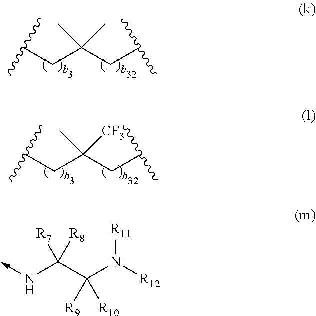

The present invention relates to compounds of formula (I), pharmaceutically acceptable salts thereof as well as pharmaceutical compositions comprising the same and, in particular, to the compounds of formula (I), pharmaceutically acceptable salts thereof as well as pharmaceutical compositions comprising the same for the use in methods of topical treatment of skin cancer including basal cell carcinoma, cutaneous squamous cell carcinoma and melanoma.

### RELATED ART

The skin is recognized as an interesting route for drug delivery since topical administration of drugs for dermal and/or transdermal applications not only avoids the risks of toxicity and the gastrointestinal side-effects often associated with other classical treatments such as systemic treatments which may be parenteral or oral but further it has greater acceptability from patients and does not require of valuations before, during or after treatment to check toxicity levels of the liver, kidney and the like. Therefore, the development of drugs suitable for topical applications in particular application to the skin receives a great deal of interest.

The human skin is the largest and most accessible organ for drug delivery. However, it represents a natural physicochemical barrier of our bodies and as a result, it is characterized by low permeability, in order to limit the transport of most pathogens, toxins and drug molecules. Thus, drug delivery into and across the skin is a serious challenge.

The main transport barrier for drug delivery into or across the skin is the stratum corneum which is typically 10-20 μm thick and consists of 10-15 layers of corneocytes surrounded by a lipid-enriched lipid matrix composed of ceramides, cholesterol and free fatty acids. To penetrate through the stratum corneum drugs must navigate through the tortuous lipid pathways surrounding the keratin-rich cells, or repeatedly partition between the aqueous, keratin-rich phase and the lipid phase. In general, small molecules are able to penetrate the stratum corneum while, in contrast, the delivery of larger molecules, such as peptides and proteins, remains a major challenge. For pharmaceutical purposes, it has been argued to restrict the development of new compounds to a molecular weight (MW) of under 500 Dalton when topical dermatological therapy or percutaneous systemic therapy is addressed since around 500 Dalton is the start of a rapid decline in skin absorption due to molecular size. This is nowadays known as the “500 Dalton rule”. Further physicochemical properties of a topical drug beneficial to achieve passive transportation through the stratum corneum are a partition coefficient octanol/water log P between 1 and 3 and a water solubility greater than 1 mg/ml and the absence of polar centers (Bos and Meinardi, Exp Dermatol 2000, 9:165-169; Gorouhi and Maibach, International Journal of Cosmetic Science, 2009, 31, 327-345).

Most skin cancers are locally destructive cancerous (malignant) growth of the skin. They originate from the cells of the epidermis, the superficial layer of the skin. Unlike cutaneous malignant melanoma, the vast majority of these sorts of skin cancers rarely spread to other parts of the body (metastasize) and become life-threatening. There are three major types of skin cancer: (1) basal cell carcinoma (BCC; the most common), (2) cutaneous squamous cell carcinoma (cSCC; the second most common), which originate from skin cells, and (3) melanoma, which originates from the pigment-producing skin cells (melanocytes) but is less common, though more dangerous, than the first two varieties. Other rare forms of skin cancer include lymphomas, Merkel cell cancers, and cancers of other tissue in the skin, including sarcomas as well as hair and sweat gland tumours (Ascierto P A, Garbe C, Immunotherapy (2020) 12(3):167-174; U. Hillen et al., European Journal of Cancer (2018) 96:34-43, Loh T Y et al., Dermatol Surg. (2017) 43(1):32-39).

### SUMMARY OF THE INVENTION

We have surprisingly found that the inventive compounds are suitable for topical application as potential pharmaceutical products for the treatment of skin cancer. In particular, it has not only been found that the inventive compounds are able to penetrate into skin as shown by pig skin permeating experiments but, in addition, are active against cutaneous squamous cell cancer, basal cell cancer and melanoma as shown by in vitro assays. Moreover, a preferred inventive compound shows successful treatment and anti-proliferative effects in an in vivo mouse model of melanoma using C67BLC/6 mice bearing B16-F10 cells tumours, the most used metastatic melanoma model for preclinical studies, and the topical drug treatment led to a significant reduction of the tumor volume in the treated group as compared to the control. Histological studies further exhibit strong proapoptotic potential. Furthermore, in ex vivo experiments using cancer tissue of several patients having basal cell carcinoma and cutaneous squamous cell carcinoma and treated with the inventive compounds led to significant reductions of proliferative cells at concentrations at as low as 500 nM.

Thus, in a first aspect, the present invention provides a compound of formula (I) for use in a method of topical treatment of a skin cancer of a mammal, preferably of a human, wherein said method comprises topical administration of said compound to said mammal, preferably to said human,

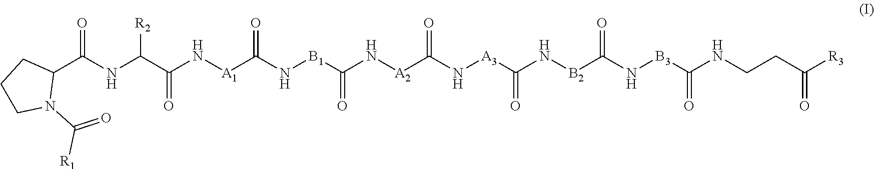

wherein
A₁ is

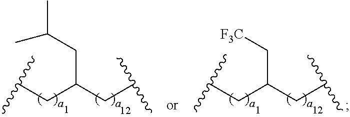

A₂ is

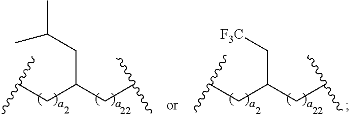

A₃ is

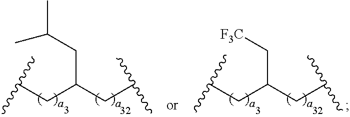

B₁ is

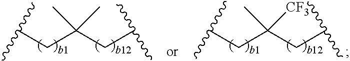

B₂ is

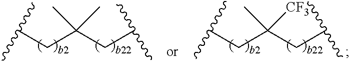

B₃ is

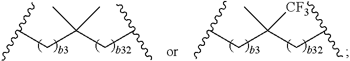

and wherein
a₁, a₁₂, a₂, a₂₂, a₃, a₃₂, b₁, b₁₂, b₂, b₂₂, b₃ and b₃₂ are independently selected from 0 and 1, wherein a₁+a₁₂, a₂+a₂₂, a₃+a₃₂, b₁+b₁₂, b₂+b₂₂, and b₃+b₃₂ are independently selected from 0 and 1, and wherein a₁+a₁₂+a₂+a₂₂+a₃+a₃₂ is 0, 1, 2 or 3, and wherein b₁+b₁₂+b₂+b₂₂+b₃+b₃₂ is 0, 1 or 2;
and wherein
R₁ is selected from carbocyclyl or heterocyclyl, each independently optionally and preferably substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl;
R₂ is selected from C₄-C₁₂alkyl, C₄-C₁₀alkoxy, C₁-C₃alkylene-cycloalkyl, C₁-C₃alkylene-aryl, C₁-C₃alkylene-heteroaryl, wherein independently in said C₁-C₃alkylene one —CH₂— moiety is optionally replaced by —CH(NH)— or —O—; and wherein said alkyl, cycloalkyl, aryl and heteroaryl are each independently optionally and preferably substituted with one or more, preferably one or two, substituents selected from C₁-C₂alkyl, C₁-C₂haloalkyl, oxo, OH, halogen, C₁-C₂alkoxy, C₆H₅ or C₆H₅ substituted with C₁-C₃alkyl or OC₁-C₃alkyl;
R₃ is

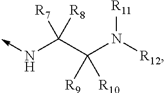

wherein
   R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a carbocyclic or heterocyclic ring, preferably a carbocyclic ring, and wherein
      R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a heteroaryl or a heterocyclyl, each independently optionally and preferably substituted with halogen, C₁-C₄alkyl, OR₁₃, NR₁₄R₁₅; wherein R₁₃, R₁₄, R₁₅ are independently at each occurrence H, C₁-C₄alkyl, and wherein the arrow indicates the attachment to the C(O)-moiety depicted in formula (I);
and pharmaceutically acceptable salts of said compound of formula (I), wherein preferably said method comprises topical administration of an effective amount of said compound to said mammal, preferably to said human.

In a further aspect, the present invention provides a compound of formula (I) for use in a method of treating a skin cancer of a mammal, preferably of a human, wherein said method comprises topical administration to said mammal, preferably to said human, of a compound of formula (I), or a pharmaceutically acceptable salt thereof, as defined herein, in particular as in any one of the appended claims, wherein preferably said method comprises topical administration of an effective amount of said compound, or said pharmaceutically acceptable salt thereof, to said mammal, preferably to said human.

In a further aspect, the present invention provides a pharmaceutical composition for use in a method of topical treatment of a skin cancer of a mammal, preferably of a human, wherein said pharmaceutical composition comprises a compound of formula (I), or a pharmaceutically acceptable salt thereof, as defined herein, in particular as in any one of the appended claims, and a pharmaceutically acceptable carrier or adjuvant, and wherein said method comprises topical administration of said pharmaceutical composition to said mammal, preferably to said human, and wherein preferably said pharmaceutical composition comprises an effective amount of said compound.

Thus, in particular, the present invention provides compounds and pharmaceutical compositions for use in methods of topical treatment of skin cancer of a mammal, preferably of a human, in particular for use in methods of topical treatment of melanoma and non-melanoma skin cancer, and preferably for melanoma, basal cell carcinoma and cutaneous squamous cell carcinoma, wherein said methods comprise topical administration of an effective amount of a compound of formula (I), or a pharmaceutically acceptable salt thereof, or a pharmaceutical composition comprising said compound of formula (I), or a pharmaceutically acceptable salt thereof, to said mammal, preferably to said human.

In another aspect, the present invention provides a method for topically treating a skin cancer of a mammal, preferably of a human, wherein said method comprises topical administration of a compound of formula (I) of the present invention, or a pharmaceutically acceptable salt thereof, or a pharmaceutical composition comprising said compound of formula (I), or a pharmaceutically acceptable salt thereof, to said mammal, preferably to said human, and wherein preferably said method comprises topical administration of an effective amount of said compound, or said pharmaceutically acceptable salt thereof, or said pharmaceutical composition to said mammal, preferably to said human.

In a further aspect, the present invention provides for the use of a compound of formula (I) of the present invention, or a pharmaceutically acceptable salt thereof, or a pharmaceutical composition comprising said compound of formula (I), or a pharmaceutically acceptable salt thereof, for the preparation of a medicament for topically treating a skin cancer of a mammal, preferably of a human, wherein said method comprises topical administration of said compound of formula (I), or said pharmaceutically acceptable salt thereof, or said pharmaceutical composition of the invention, to said mammal, preferably to said human, and wherein preferably said method comprises topical administration of an effective amount of said compound, or said pharmaceutically acceptable salt thereof, or said pharmaceutical composition, to said mammal, preferably to said human.

Further aspects and embodiments of the present invention will be become apparent as this description continues.

### DESCRIPTION OF FIGURES

FIG. 1A: Tumour slices taken from C57Bl/6 mice bearing B16F10 xenografts were harvested and studied histologically. Tumour slices were stained with proliferation marker Ki67 to identify tumour regions with proliferating cells and with apoptosis marker TUNEL to show areas within tumours with apoptotic cells.

FIG. 1B: Quantification of the Ki67 and TUNEL positively stained cells as described in FIG. 1A for group A (placebo) and for group B (6027).

FIG. 2A: Immunohistochemistry stainings of patient derived Basal Cell Carcinoma (BCC) which was cultivated for 4 days in medium containing 500 nM and 1 μM of 6027 or DMSO as negative control and Vismodegib (20 μM) as positive control.

FIG. 2B: Quantification of the Ki67 and TUNEL positively stained cells as described in FIG. 2A for A (DMSO), B (Vismodegib, 20 mM), C (6027, 500 nM) and D (6027, 1 mM).

FIG. 3A: Immunohistochemistry stainings of patient derived Basal Cell Carcinoma (BCC) which was cultivated for 4 days in medium containing 500 nM of 6027 or DMSO as negative control and Vismodegib (20 μM) as positive control.

FIG. 3B: Quantification of the Ki67 and TUNEL positively stained cells as described in FIG. 3A for A (DMSO), B (Vismodegib, 20 mM) and C (6027, 500 nM).

FIG. 4A: Histopathological slices of patient derived basal cell carcinoma (BCC) which was cultivated for 4 days with 500 nM of 6027 as well as with standard of care Vismodegib (20 μM) as positive control.

FIG. 4B: Quantification of the Ki67 positively stained cells as described in FIG. 4A for A (Vismodegib, 20 mM) and B (6027, 500 nM).

FIG. 5A: Histopathological slices of patient derived high grade cutaneous squamous cell carcinoma (cSCC) which was treated for 4 days with 500 nM of 6027 and DMSO as negative control.

FIG. 5B: Quantification of the Ki67 positively stained cells as described in FIG. 5A for A (DMSO) and B (6027, 500 nM).

### DETAILED DESCRIPTION OF THE INVENTION

Unless defined otherwise, all technical and scientific terms used herein have the same meanings as commonly understood by one of ordinary skill in the art to which this invention belongs. The herein described and disclosed embodiments, preferred embodiments and very preferred embodiments should apply to all aspects and other embodiments, preferred embodiments and very preferred embodiments irrespective of whether is specifically again referred to.

When the terms “a,” or “an” are used herein, they mean “at least one” unless indicated otherwise.

Each alkyl moiety either alone or as part of a larger group such as alkoxy, aminoalkyl or haloalkoxy or alkylene refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₆alkyl” denotes an alkyl group having 1 to 6 carbon atoms. Examples include methyl, ethyl, n-propyl, prop-2-yl, n-butyl, but-2-yl, 2-methyl-prop-1-yl or 2-methyl-prop-2-yl. Examples of an alkoxy include methoxy, ethoxy, propoxy, iso-propoxy, n-butoxy, sec-butoxy, tert-butoxy, n-pentoxy, neo-pentoxy, n-hexoxy. Examples of aminoalkyl include aminomethyl, aminoethyl, dimethylaminomethyl, dimethylaminoethyl. Haloalkoxy refers to alkoxy with further substitution of halogen.

As used herein, the term “alkylene” refers to an alkanediyl group, i.e. a divalent saturated acyclic hydrocarbon group which may be linear or branched. A “C₁₋₆alkylene” denotes an alkylene group having 1 to 6 carbon atoms. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—).

Each haloalkyl moiety either alone or as part of a larger group such as haloalkoxy is an alkyl group substituted by one or more of the same or different halogen atoms. Haloalkyl include for example 1 to 5 halo substituents, or 1 to 3 halo substituents. Examples include in particular fluoromethyl, difluoromethyl, trifluoromethyl, chlorodifluoromethyl and 2,2,2-trifluoro-ethyl.

Each alkenyl moiety either alone or as part of a larger group such as alkenyloxy or alkenylene is a straight or branched chain and is preferably C₂-C₁₆alkenyl, more preferably C₂-C₁₄alkenyl. Each moiety can be of either the (E)- or (Z)-configuration. Examples include vinyl and allyl. A compound of the present invention comprising an alkenyl moiety thus may include, if applicable, either said compound with said alkenyl moiety in its (E)-configuration, said compound with said alkenyl moiety in its (Z)-configuration and mixtures thereof in any ratio.

Halogen is fluorine, chlorine, bromine, or iodine.

As used herein, the term “carbocyclyl” refers to a monovalent hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl. The number of carbon atoms in the carbocyclyl group is not particularly limited and is preferably 3 to 14, more preferably 4 to 12 or 5 to 10.

As used herein, the term “heterocyclyl” refers to a monovalent ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. Unless defined otherwise, “heterocyclyl” preferably refers to heteroaryl, heterocycloalkyl or heterocycloalkenyl. The number of carbon atoms in the heterocyclyl group is not particularly limited and is preferably 5 to 14, more preferably 5 to 12 or 5 to 10.

The term “aryl”, as used herein, refers to a monovalent aromatic hydrocarbon radical of 6-14 carbon atoms (C₆-C₁₄). Aryl includes bicyclic, tricyclic or tetracyclic, preferably bicyclic, radicals comprising an aromatic ring to which saturated, partially unsaturated or aromatic carbocyclic or heterocyclic ring(s) are fused or bridged. Aryl groups are optionally substituted independently with one or more substituents, typically and preferably with one or two substituents, wherein said substituents are typically and preferably independently at each occurrence selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂. Typical aryl groups include, but are not limited to, phenyl, substituted phenyls, naphthyl, 1,2-dihydronapthalenyl, 1,2,3,4-tetrahydronaphthenyl, anthracenyl, phenanthrenyl, biphenyl, indenyl and indanyl. Unless defined otherwise, an “aryl” preferably has 5 to 14 ring atoms, more preferably 5 to 10 ring atoms, and most preferably refers to phenyl or phenyl substituted by one or two substituents, preferably by one substituent, independently selected from C₁-C₄alkyl, halogen, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). Heteroaryl groups are optionally substituted independently with one or more substituents, typically and preferably with one or two substituents independently selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂. Unless defined otherwise, a “heteroaryl” preferably has 5 to 14 ring atoms, more preferably 5 to 12 or 5 to 10 ring atoms.

In a preferred embodiment, said heteroaryl is a monovalent monocyclic aromatic or bicyclic aromatic ring group, wherein said aromatic ring group comprises one or more (such as, e.g., one, two, three, or four) ring heteroatoms independently selected from O, S and N, and the remaining ring atoms are carbon atoms, and wherein said aromatic ring group is optionally substituted independently with one or more substituents, typically and preferably with one or two substituents independently selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂. Unless defined otherwise, such a monocyclic or bicyclic heteroaryl preferably has 5 to 12, preferably 5 to 10 ring atoms.

Examples of heteroaryl groups are pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, thiadiazolyl, furazanyl, benzofurazanyl, benzopyranyl, benzothiophenyl, benzothiazolyl, benzooxazolyl, coumarinyl, quinazolinyl, quinoxalinyl, naphthyridinyl, and furopyridinyl, wherein said heteroaryl are optionally substituted independently with one or more, preferably one or two substituents independently selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂.

Further examples of such monocyclic heteroaryl radicals include, but are not limited to: 2-pyridyl, 3-pyridyl, 4-pyridyl, 3-isoxazolyl, 4-isoxazolyl, 5-isoxazolyl, 2-imidazolyl, 4-imidazolyl, 3-pyrazolyl, 4-pyrazolyl, 2-pyrrolyl, 3-pyrrolyl, 2-thiazolyl, 4-thiazolyl, 5-thiazolyl, 3-pyridazinyl, 4-pyridazinyl, 5-pyridazinyl, 2-pyrimidinyl, 5-pyrimidinyl, 6-pyrimidinyl, 2-pyrazinyl, 2-oxazolyl, 4-oxazolyl, 5-oxazolyl, 2-furanyl, 3-furanyl, 2-thienyl, 3-thienyl, 3-triazolyl, 1-triazolyl, 5-tetrazolyl, 1-tetrazolyl, and 2-tetrazolyl, which are optionally substituted independently with one or more substituents, typically and preferably with one or two substituents, wherein said substituents are independently at each occurrence independently selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂.

As used herein, the term “cycloalkyl” refers to a monovalent saturated hydrocarbon ring group, including monocyclic rings as well as bridged ring, spiro ring and/or fused ring systems (which may be composed, e.g., of two, three or four rings; such as, e.g., a fused ring system composed of two or three fused rings). Cycloalkyl groups are optionally substituted independently with one or more substituents, typically and preferably with one or two substituents independently selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂. Unless defined otherwise, “cycloalkyl” preferably refers to a C₃₋₁₄ cycloalkyl. Examples of cycloalkyl are cyclopropyl, cyclobutyl, cyclopentyl, cyclohexyl, cycloheptyl, or adamantyl.

As used herein, the term “heterocycloalkyl” refers to a monovalent saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). Heterocycloalkyl groups are optionally substituted independently with one or more substituents, typically and preferably with one or two substituents independently selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂. Unless defined otherwise, “heterocycloalkyl” preferably refers to a 3 to 14 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. Cycloalkenyl groups are optionally substituted independently with one or more substituents, typically and preferably with one or two substituents, wherein said substituents are typically and preferably independently at each occurrence selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₄cycloalkenyl, and more preferably refers to a C₃₋₇ cycloalkenyl.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms and carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. Heterocycloalkenyl groups are optionally substituted independently with one or more substituents, typically and preferably with one or two substituents, wherein said substituents are typically and preferably independently at each occurrence selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂. Unless defined otherwise, “heterocycloalkenyl” preferably refers to a 3 to 14 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

Where a group is said to be optionally substituted, preferably there are optionally 1-5 substituents, more preferably optionally 1-3 substituents, and again more preferably optionally one or two substituents.

The term “amino acid”, as used herein, refers to organic compounds containing the functional groups amine (—NH₂) and carboxylic acid (—COOH) and its zwitterions, typically and preferably, along with a side chain specific to each amino acid. The term “amino acid” typically and preferably includes amino acids that occur naturally, such as proteinogenic amino acids (produced by RNA-translation), non-proteinogenic amino acids (produced by other metabolic mechanisms, e.g. posttranslational modification), standard or canonical amino acids (that are directly encoded by the codons of the genetic code) and non-standard or non-canonical amino acids (not directly encoded by the genetic code). Naturally occurring amino acids include non-eukaryotic and eukaryotic amino acids. The term “amino acid”, as used herein, also includes unnatural amino acids that are chemically synthesized. Moreover, the term covers alpha- (α-), beta- (β-), gamma- (γ-) and delta- (δ-) etc. amino acids as well as mixtures thereof in any ratio, and any isomeric form of an amino acid, i.e. D- and L-stereoisomers (alternatively addressed by the (R) and (S) nomenclature) as well as mixtures thereof in any ratio, preferably in a racemic ratio of 1:1. Amino acids in this invention are preferably in L-configuration. The term “D-stereoisomer”, “L-stereoisomer”, “D-amino acid” or “L-amino acid” refers to the chiral alpha carbon of the amino acids.

Certain compounds of formula (I) of the present invention may contain one or two or more centers of chirality and such compounds may be provided as pure enantiomers or pure diastereoisomers as well as mixtures thereof in any ratio. The compounds of the invention also include all tautomeric forms of the compounds of formula (I). The compounds of formula (I) may also be solvated, especially hydrated, which are also included in the compounds of formula (I). The term “chiral” refers to compounds, which have the property of non-superimposability of the mirror image partner, while the term “achiral” refers to compounds, which are superimposable on their mirror image partner. The term “stereoisomers” refers to compounds, which have identical chemical constitution, but differ with regard to the arrangement of the atoms or groups in space. “Diastereomer” refers to a stereoisomer with two or more centers of chirality in which the compounds are not mirror images of one another. Diastereomers have different physical properties, e.g. melting points, boiling points, spectral properties, and chemical and biological reactivities. Mixtures of diastereomers may be separated under high resolution analytical procedures such as electrophoresis and chromatography. “Enantiomers” refer to two stereoisomers of a compound which are non-superimposable mirror images of one another. Stereochemical definitions and conventions used herein generally follow S. P. Parker, Ed., McRaw-Hiff Dictionary of Chemical Terms (1984), McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric or a scalemic mixture. A 50:50 mixture of enantiomers is referred to as a racemic mixture or a racemate. The term “tautomer” or “tautomeric form” refers to structural isomers of different energies, which are interconvertible via a low energy barrier. For example, proton tautomers include interconversions via migration of a proton, such as keto-enol and imine-enamine isomerizations.

The phrase “pharmaceutically acceptable salt” as used herein, refers to pharmaceutically acceptable organic or inorganic salts of a compound of the invention, in particular acid addition salts. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate (mesylate), ethanesulfonate, benzenesulfonate, p-toluenesulfonate, and pamoate salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such asp-toluenesulfonic acid or ethanesulfonic acid, or the like.

A “solvate” refers to an association or complex of one or more solvent molecules and a compound of the invention. Examples of solvents that form solvates include, but are not limited to, water, isopropanol, ethanol, methanol, dimethyl sulfoxide (DMSO), ethyl acetate, acetic acid, and ethanolamine. The term “hydrate” refers to the complex where the solvent molecule is water.

The terms “compound of this invention” and “compounds of the present invention” and “compounds of formula (I)” include stereoisomers, geometric isomers, tautomers, solvates, pharmaceutically acceptable salts, and solvates of the salts thereof.

The term “mammal” includes, but is not limited to, humans, mice, rats, guinea pigs, monkeys, dogs, cats, horses, cows, pigs, and sheep. The term “mammal”, as used herein, preferably refers to humans.

The term “treatment” of a disorder or disease as used herein (e.g., “treatment” of a skin cancer such as melanoma) is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment. The treatment of a disorder or disease may, inter alia, comprise curative treatment (preferably leading to a complete response and eventually to healing of the disorder or disease) and palliative treatment (including symptomatic relief). The “amelioration” of a disorder or disease may, for example, lead to a halt in the progression of the disorder or disease or a delay in the progression of the disorder or disease. “Treatment” can also mean prolonging survival as compared to expected survival if not receiving treatment.

The term “effective amount” means an amount of a compound of the present invention or an inventive pharmaceutical composition that (i) treats the particular disease or disorder or (ii) attenuates, ameliorates, or eliminates one or more symptoms of the particular disease or disorder. In the case of a skin cancer, in particular in case of a cancer is a melanoma, a basal cell carcinoma or a cutaneous squamous cell carcinoma, the effective amount of the drug may reduce the tumor volume and/or reduce clinical symptoms.

The term “skin cancer” as used herein, refers to a skin cancer such as a melanoma skin cancer and non-melanoma skin cancer (NMSC) but also refers and includes precancer and pre-invasive neoplastic skin proliferations such as cutaneous squamous cell carcinoma in situ (cSCCis or Bowen's disease) or Actinic Keratosis (AK, also called “solar keratosis” and “senile keratosis”). Moreover, the term “skin cancer” as used herein, shall also include cutaneous lymphomas such as cutaneous T-cell lymphoma (CTCL) or cutaneous B-cell lymphoma (CBCL), or a pre-invasive form thereof. Bowen's disease is a neoplastic skin disease which can be considered as an early stage or intraepidermal form of squamous cell carcinoma. Actinic keratosis is characterized by pre-cancerous patches of thick, scaly, or crusty skin, which are usually formed when skin gets damaged by ultraviolet (UV) radiation from the sun or indoor tanning beds.

The term “non-melanoma skin cancer”, (abbreviated as “NMSC”) is used herein to refer to a group of diseases in particular including cutaneous squamous cell carcinoma (cSCC), basal cell carcinoma (BCC), Merkel Cell carcinoma (MCC), Bowen's Disease (BD) and Actinic Keratosis (AK), as well as any precancer and pre-invasive forms thereof.

The term “field cancerization” refers to premalignant field defects and is a biological process in which large areas of cells at a tissue surface or within an organ are affected by carcinogenic alterations. The process arises from exposure to an injurious environment, such as UV radiation, often over a lengthy period. The initial step in field cancerization is associated with various molecular lesions such as acquired genetic mutations and epigenetic changes, occurring over a widespread, multi-focal “field”. The field is affected by subclinical (nonvisible, nonpalpable) AK lesions, early AK lesions, late AK lesions, and possibly even invasive cSCCs. The concept of field cancerization provides the rationale for field therapy, in which the entire field—rather than individual lesions—is treated. The goals of field therapy are to eliminate not only clinically visible lesions but also subclinical lesions and to prevent the development of invasive SCC.

The term “topical administration” as used herein, refers to application to body surfaces such as the skin. Typically and preferably, the term “topical administration” as used herein, refers to epicutaneous application meaning application directly to the skin. Thus, in a very preferred embodiment of the present invention, said topical administration is application on the skin, wherein preferably said topical administration is application directly on the skin. Typically and preferably, the present invention relates to locally applied and locally acting compounds for cutaneous use to cure infections of the skin, as referred to in the quality-equivalence guidelines for topical products of EMA (https://www.ema.europa.eu/en/documents/scientific-guideline/draft-guideline-quality-equivalence-topical-products_en.pdf).

In a first aspect, the present invention provides a compound of formula (I) for use in a method of topical treatment of a skin cancer of a mammal, wherein said method comprises topical administration of said compound to said mammal,

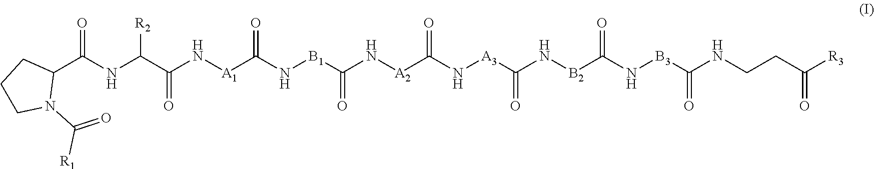

wherein
A₁ is

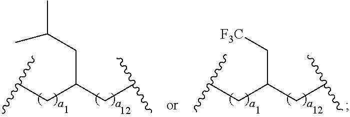

A₂ is

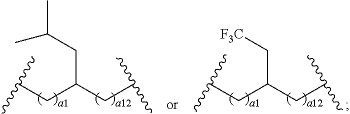

A₃ is

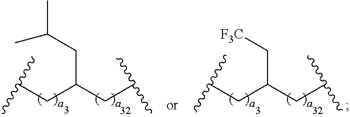

B₁ is

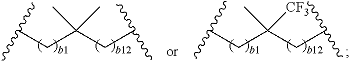

B₂ is

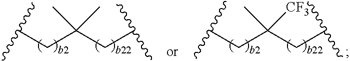

B₃ is

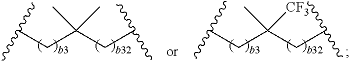

and wherein
a₁, a₁₂, a₂, a₂₂, a₃, a₃₂, b₁, b₁₂, b₂, b₂₂, b₃ and b₃₂ are independently selected from 0 and 1, wherein a₁+a₁₂, a₂+a₂₂, a₃+a₃₂, b₁+b₁₂, b₂+b₂₂, and b₃+b₃₂ are independently selected from 0 and 1, and wherein a₁+a₁₂+a₂+a₂₂+a₃+a₃₂ is 0, 1, 2 or 3, and wherein b₁+b₁₂+b₂+b₂₂+b₃+b₃₂ is 0, 1 or 2;
and wherein
R₁ is selected from carbocyclyl or heterocyclyl, each independently optionally and preferably substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl;
R₂ is selected from C₄-C₁₂alkyl, C₄-C₁₀alkoxy, C₁-C₃alkylene-cycloalkyl, C₁-C₃alkylene-aryl, C₁-C₃alkylene-heteroaryl, wherein independently in said C₁-C₃alkylene one —CH₂— moiety is optionally replaced by —CH(NH)— or —O—; and wherein said alkyl, cycloalkyl, aryl and heteroaryl are each independently optionally and preferably substituted with one or more, preferably one or two, substituents selected from C₁-C₂alkyl, C₁-C₂haloalkyl, oxo, OH, halogen, C₁-C₂alkoxy, C₆H₅ or C₆H₅ substituted with C₁-C₃alkyl or OC₁-C₃alkyl;
R₃ is

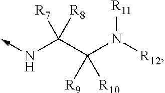

wherein
   R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a carbocyclic or heterocyclic ring, preferably a carbocyclic ring, and wherein
      R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a heteroaryl or a heterocyclyl, each independently optionally and preferably substituted with halogen, C₁-C₄alkyl, OR₁₃, NR₁₄R₁₅; wherein R₁₃, R₁₄, R₁₅ are independently at each occurrence H, C₁-C₄alkyl, and wherein the arrow indicates the attachment to the C(O)-moiety depicted in formula (I);
and pharmaceutically acceptable salts of said compound of formula (I), wherein preferably said method comprises topical administration of an effective amount of said compound to said mammal, preferably to a human.

In a further aspect, the present invention provides a compound of formula (I*) for use in a method of topically treating a skin cancer of a mammal, wherein said method comprises topical administration of said compound to said mammal, preferably to a human,

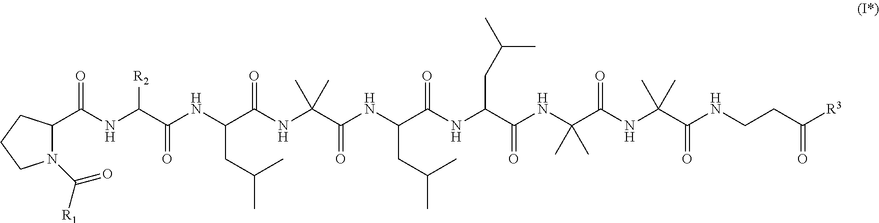

wherein
R₁ is selected from carbocyclyl or heterocyclyl, each independently optionally and preferably substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl;
R₂ is selected from C₅-C₁₂alkyl, C₄-C₁₀alkoxy, C₁-C₃alkylene-cycloalkyl, C₁-C₃alkylene-aryl, C₁-C₃alkylene-heteroaryl, wherein independently in said C₁-C₃alkylene one —CH₂— moiety is optionally replaced by —CH(NH)— or —O—; and wherein said alkyl, cycloalkyl, aryl and heteroaryl are each independently optionally and preferably substituted with one or more, typically and preferably, one or two substituents selected from C₁-C₂alkyl, C₁-C₂haloalkyl, oxo, OH, halogen, C₁-C₂alkoxy, C₆H₅ or C₆H₅ substituted with C₁-C₃alkyl or OC₁-C₃alkyl;
R₃ is

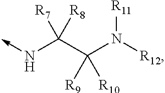

wherein
   R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a carbocyclic or heterocyclic ring, preferably a carbocyclic ring, and wherein
      R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a heteroaryl or a heterocyclyl, each independently optionally and preferably substituted with halogen, C₁-C₄alkyl, OR₁₃, NR₁₄R₁₅; wherein R₁₃, R₁₄, R₁₅ are independently at each occurrence H, C₁-C₄alkyl, and wherein the arrow indicates the attachment to the C(O)-moiety depicted in formula (I);
and pharmaceutically acceptable salts of said compound of formula (I*), wherein preferably said method comprises topical administration of an effective amount of said compound to said mammal, preferably to a human.

In another aspect, the present invention provides a method for topically treating a skin cancer of a mammal, preferably of a human, wherein said method comprises topical administration of a compound of formula (I), or a pharmaceutically acceptable salt thereof, or a pharmaceutical composition comprising said compound of formula (I), or a pharmaceutically acceptable salt thereof, to said mammal, preferably to said human, and wherein preferably said method comprises topical administration of an effective amount of said compound to said mammal, preferably to said human.

In a further aspect, the present invention provides for the use of a compound of formula (I) of the present invention, or a pharmaceutically acceptable salt thereof, or a pharmaceutical composition comprising said compound of formula (I), or a pharmaceutically acceptable salt thereof, for the preparation of a medicament for topically treating a skin cancer of a mammal, preferably of a human, wherein said method comprises topical administration of said compound of formula (I), or said pharmaceutically acceptable salt thereof, or said pharmaceutical composition of the invention, to said mammal, preferably to said human, and wherein preferably said method comprises topical administration of an effective amount of said compound, or said pharmaceutically acceptable salt thereof, or said pharmaceutical composition, to said mammal, preferably to said human.

In a preferred embodiment, said compound of formula (I) is a compound of any one of the formula (II) to (IV)

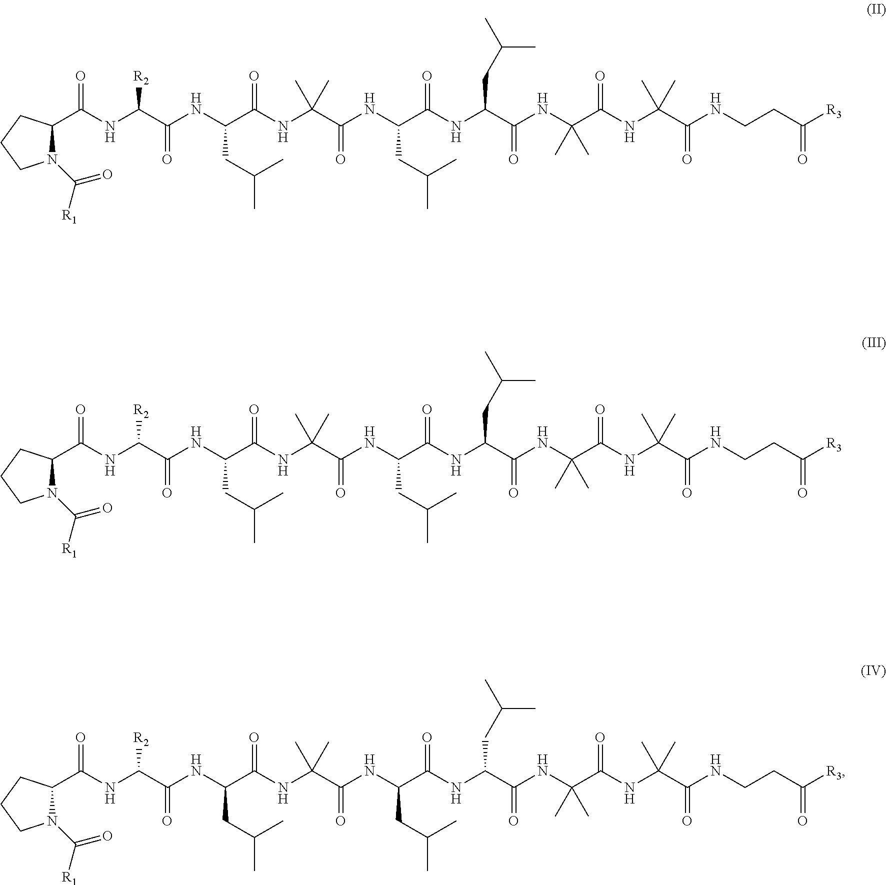

wherein preferably said compound of formula (I) is a compound of formula (II).

In another preferred embodiment, said compound of formula (I) is a compound of formula (II), formula (III) or formula (IV), wherein preferably said compound of formula (I) is a compound of formula (II). In another preferred embodiment, said compound of formula (I) is a compound of formula (II). In another preferred embodiment, said compound of formula (I) is a compound of formula (III). In another preferred embodiment, said compound of formula (I) is a compound of formula (IV).

In a further preferred embodiment, said R₁ is selected from phenyl, naphthyl, 1,2-dihydronapthalenyl, 1,2,3,4-tetrahydronaphthenyl, anthracenyl, phenanthrenyl, biphenyl, indenyl, indanyl, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, thiadiazolyl, furazanyl, benzofurazanyl, benzopyranyl, benzothiophenyl, benzothiazolyl, benzooxazolyl, coumarinyl, quinazolinyl, quinoxalinyl, naphthyridinyl, furopyridinyl, cyclopentyl, cyclopentenyl, cyclohexyl, cyclohexenyl, adamantanyl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In another preferred embodiment, said R₁ is selected from cycloalkyl, aryl or heteroaryl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from cycloalkyl, monocyclic or bicyclic aryl or monocyclic or bicyclic heteroaryl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from cycloalkyl, monocyclic or bicyclic aromatic aryl or monocyclic or bicyclic heteroaryl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl or a monocyclic or bicyclic heteroaryl comprising one or two heteroatoms selected from N, O and S, preferably from phenyl, coumarinyl or oxazolyl, further preferably phenyl or oxazolyl; each independently optionally substituted with C₁-C₄alkyl, halogen, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl In a further preferred embodiment, said R₁ is selected from phenyl, naphthyl, 1,2-dihydronapthalenyl, 1,2,3,4-tetrahydronaphthenyl, biphenyl, indenyl, indanyl, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, thiadiazolyl, furazanyl, benzofurazanyl, benzopyranyl, benzothiophenyl, benzothiazolyl, benzooxazolyl, coumarinyl, quinazolinyl, quinoxalinyl, naphthyridinyl, furopyridinyl, cyclopentyl, cyclohexyl, adamantanyl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from the formula

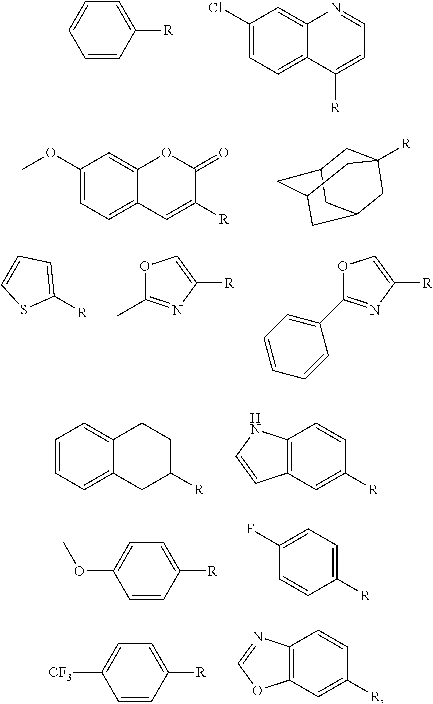

wherein R indicates the attachment to the C(O)-moiety depicted in formula (I).

In a further preferred embodiment, said R₁ is selected from phenyl or monocyclic or bicyclic heteroaryl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl. In a further preferred embodiment, said R₁ is selected from phenyl or monocyclic or bicyclic aromatic heteroaryl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, thiadiazolyl, benzofurazanyl, benzopyranyl, benzothiophenyl, benzothiazolyl, benzooxazolyl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl, imidazolyl, furyl, thienyl, isoxazolyl, thiazolyl, oxazolyl, isothiazolyl, pyrrolyl, quinolinyl, isoquinolinyl, indolyl, benzimidazolyl, benzopyranyl, benzothiophenyl, benzothiazolyl, benzooxazolyl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl, imidazolyl, furyl, thienyl, isoxazolyl, thiazolyl, oxazolyl, isothiazolyl, pyrrolyl, quinolinyl, isoquinolinyl, indolyl, benzimidazolyl, benzopyranyl, benzothiophenyl, benzothiazolyl, benzooxazolyl, coumarinyl, each independently optionally substituted with methyl, ethyl, chlorine, fluorine, oxo, CF₃, OC₁-C₂alkyl, NR₁₁R₁₂, C₆H₅, C₆H₅ substituted with methyl, ethyl, chlorine, fluorine, OC₁-C₂alkyl, NR₁₁R₁₂, wherein R₁₁, R₁₂ are independently at each occurrence H, methyl, ethyl.

In a further preferred embodiment, said R₁ is selected from the formula

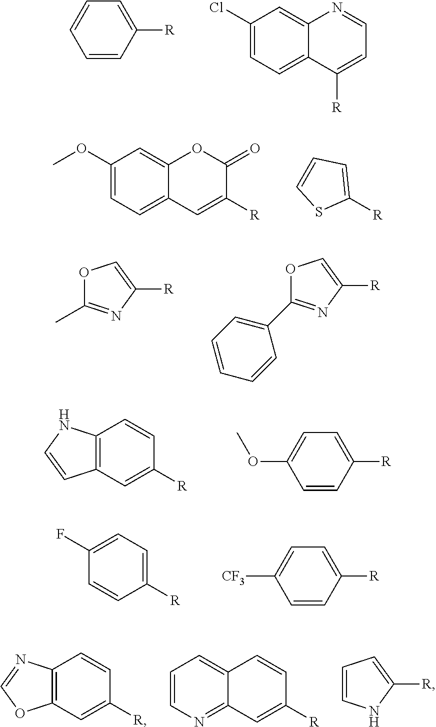

wherein R indicates the attachment to the C(O)-moiety depicted in formula (I).

In a further preferred embodiment said R₁ is selected from phenyl or a monocyclic or bicyclic heteroaryl, each independently optionally substituted with C₁-C₄alkyl, halogen, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl or a monocyclic or bicyclic heteroaryl, each independently optionally substituted, preferably mono-substituted, with methyl, ethyl, chlorine, fluorine, CF₃, OC₁-C₂alkyl, NR₅R₆, C₆H₅, C₆H₅ substituted with methyl, ethyl, chlorine, fluorine, OC₁-C₂alkyl, NR₅R₆, wherein R₅, R₆ are independently at each occurrence H, methyl, ethyl.

In a further preferred embodiment said R₁ is selected from phenyl or a monocyclic or bicyclic heteroaryl comprising one or two heteroatoms selected from N, O and S; each independently optionally substituted with C₁-C₄alkyl, halogen, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl or a monocyclic or bicyclic heteroaryl comprising one or two heteroatoms selected from N, O and S; each independently optionally substituted, preferably mono-substituted, with methyl, ethyl, chlorine, fluorine, CF₃, OC₁-C₂alkyl, NR₅R₆, C₆H₅, C₆H₅ substituted with methyl, ethyl, chlorine, fluorine, OC₁-C₂alkyl, NR₅R₆, wherein R₅, R₆ are independently at each occurrence H, methyl, ethyl.

In a further preferred embodiment, said R₁ is selected from phenyl, imidazolyl, furyl, thienyl, isoxazolyl, thiazolyl, oxazolyl, isothiazolyl, pyrrolyl, indolyl, coumarinyl, each independently optionally substituted with C₁-C₄alkyl, halogen, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl, imidazolyl, furyl, thienyl, isoxazolyl, thiazolyl, oxazolyl, isothiazolyl, pyrrolyl, indolyl, coumarinyl, each independently optionally substituted, preferably mono-substituted, with methyl, ethyl, chlorine, fluorine, CF₃, OC₁-C₂alkyl, NR₅R₆, C₆H₅, C₆H₅ substituted with methyl, ethyl, chlorine, fluorine, OC₁-C₂alkyl, NR₅R₆, wherein R₅, R₆ are independently at each occurrence H, methyl, ethyl.

In a further preferred embodiment, said R₁ is selected from phenyl, thienyl, oxazolyl, pyrrolyl, coumarinyl, each independently optionally substituted with C₁-C₄alkyl, halogen, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl, thienyl, oxazolyl, pyrrolyl, coumarinyl, each independently optionally substituted, preferably mono-substituted, with methyl, ethyl, chlorine, fluorine, CF₃, OC₁-C₂alkyl, NR₅R₆, C₆H₅, C₆H₅ substituted with methyl, ethyl, chlorine, fluorine, OC₁-C₂alkyl, NR₅R₆, wherein R₅, R₆ are independently at each occurrence H, methyl, ethyl.

In a further preferred embodiment, said R₁ is selected from the formula

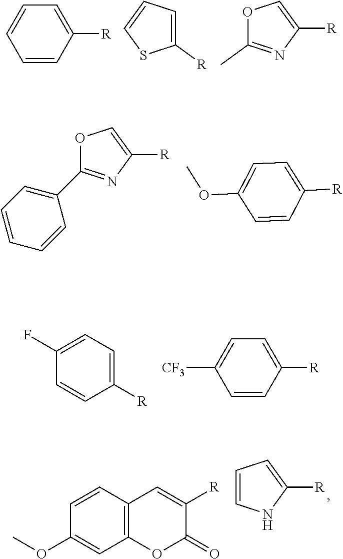

wherein R indicates the attachment to the C(O)-moiety depicted in formula (I).

In a further preferred embodiment, said R₁ is phenyl or oxazolyl, each independently optionally substituted with C₁-C₄alkyl, halogen, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is phenyl, coumarinyl or oxazolyl, each independently optionally substituted, preferably mono-substituted, with methyl, ethyl, chlorine, fluorine, CF₃, OC₁-C₂alkyl, NR₅R₆, C₆H₅, C₆H₅ substituted with methyl, ethyl, chlorine, fluorine, OC₁-C₂alkyl, NR₅R₆, wherein R₁₁, R₁₂ are independently at each occurrence H, methyl, ethyl.

In a further preferred embodiment, said R₁ is phenyl, coumarinyl or oxazolyl, each independently optionally substituted, preferably mono-substituted, with methyl, chlorine, fluorine, CF₃, OCH₃ C₆H₅, C₆H₅ substituted, preferably mono-substituted, with methyl or fluorine.

In a further very preferred embodiment, said R₁ is selected from the formula

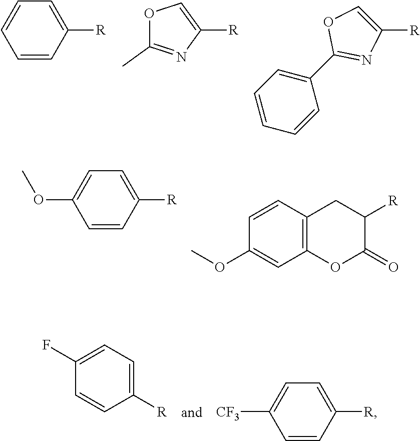

wherein R indicates the attachment to the C(O)-moiety depicted in formula (I).

In a further very preferred embodiment, said R₁ is

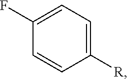

wherein R indicates the attachment to the C(O)-moiety depicted in formula (I).

In a another very preferred embodiment, said R₁ is w

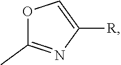

wherein R indicates the attachment to the C(O)-moiety depicted in formula (I).

In a further very preferred embodiment, said R₁ is

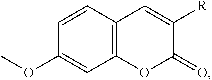

wherein R indicates the attachment to the C(O)-moiety depicted in formula (I).

In a another very preferred embodiment, said R₂ is selected from C₅-C₁₂alkyl, C₄-C₁₀alkoxy, C₁-C₃alkylene-cycloalkyl, C₁-C₃alkylene-aryl, C₁-C₃alkylene-heteroaryl, wherein independently in said C₁-C₃alkylene one —CH₂— moiety is optionally replaced by —CH(NH)— or —O—; and wherein said alkyl, cycloalkyl, aryl and heteroaryl are each independently optionally and preferably substituted with one or more, typically and preferably one or two, substituents selected from C₁-C₂alkyl, C₁-C₂haloalkyl, oxo, OH, halogen, C₁-C₂alkoxy, C₆H₅ or C₆H₅ substituted with C₁-C₃alkyl or OC₁-C₃alkyl.

In a another very preferred embodiment, said R₂ is selected from C₅-C₁₂alkyl, C₄-C₁₀alkoxy, C₁-C₃alkylene-C₅-C₆cycloalkyl, C₁-C₃alkylene-phenyl, C₁-C₃alkylene-biphenyl, C₁-C₃alkylene-(mono- or bicyclic-heteroaryl), wherein independently in said C₁-C₃alkylene one —CH₂— moiety is optionally replaced by —CH(NH)— or —O—, and wherein said phenyl, biphenyl, C₅-C₆-cycloalkyl, and mono- or bicyclic-heteroaryl are each independently optionally substituted with one or more, typically and preferably one or two, substituents selected from C₁-C₂alkyl, C₁-C₂haloalkyl, halogen, C₁-C₂alkoxy, wherein preferably said mono- or bicyclic-heteroaryl is selected from imidazolyl, furyl, thienyl, isoxazolyl, thiazolyl, oxazolyl, isothiazolyl, pyrrolyl, quinolinyl, isoquinolinyl, indolyl, benzimidazolyl, benzopyranyl, benzothiophenyl, benzothiazolyl, benzooxazolyl.

In a further preferred embodiment said R₂ is selected from C₅-C₁₂alkyl, C₄-C₁₀alkoxy, C₁-C₃alkylene-C₅-C₆cycloalkyl, C₁-C₃alkylene-phenyl, C₁-C₃alkylene-biphenyl, C₁-C₃alkylene-(mono- or bicyclic-heteroaryl), wherein independently in said C₁-C₃alkylene one —CH₂— moiety is optionally replaced by —CH(NH)— or —O—, and wherein said phenyl, biphenyl, C₅-C₆-cycloalkyl, and mono- or bicyclic-heteroaryl are each independently optionally substituted with one or more, typically and preferably one or two, substituents selected from methyl, ethyl, fluorine, chlorine, methoxy, wherein preferably said mono- or bicyclic-heteroaryl is selected from imidazolyl, furyl, thienyl, isoxazolyl, thiazolyl, oxazolyl, isothiazolyl, pyrrolyl, quinolinyl, isoquinolinyl, indolyl, benzimidazolyl, benzopyranyl, benzothiophenyl, benzothiazolyl, benzooxazolyl.

In a further preferred embodiment said R₂ is selected from C₅-C₁₂alkyl, C₄-C₁₀alkoxy, C₁-C₂alkylene-C₅-C₆cycloalkyl, C₁-C₂alkylene-phenyl, C₁-C₂alkylene-biphenyl, C₁-C₂alkylene-(mono- or bicyclic-heteroaryl), wherein independently in said C₁-C₂alkylene one —CH₂— moiety is optionally replaced by —CH(NH)— or —O—, and wherein said phenyl, biphenyl, C₅-C₆-cycloalkyl, and mono- or bicyclic-heteroaryl are each independently optionally substituted with one or more, typically and preferably one or two, substituents selected from C₁-C₂alkyl, C₁-C₂haloalkyl, halogen, C₁-C₂alkoxy, wherein preferably said mono- or bicyclic-heteroaryl is selected from imidazolyl, furyl, thienyl, isoxazolyl, thiazolyl, oxazolyl, isothiazolyl, pyrrolyl, quinolinyl, isoquinolinyl, indolyl, benzimidazolyl, benzopyranyl, benzothiophenyl, benzothiazolyl, benzooxazolyl, and wherein further preferably said mono- or bicyclic-heteroaryl is selected from isoxazolyl, thiazolyl, oxazolyl, isothiazolyl, indolyl, benzimidazolyl, benzothiazolyl, benzooxazolyl.

In a further preferred embodiment said R₂ is selected from C₅-C₁₂alkyl, C₄-C₁₀alkoxy, C₁-C₂alkylene-C₅-C₆cycloalkyl, C₁-C₂alkylene-phenyl, C₁-C₂alkylene-biphenyl, C₁-C₂alkylene-(mono- or bicyclic-heteroaryl), wherein independently in said C₁-C₂alkylene one —CH₂— moiety is optionally replaced by —CH(NH)— or —O—, and wherein said phenyl, biphenyl, C₅-C₆-cycloalkyl, and mono- or bicyclic-heteroaryl are each independently optionally substituted with one or more, typically and preferably one or two, substituents selected from methyl, ethyl, fluorine, chlorine, methoxy, wherein preferably said mono- or bicyclic-heteroaryl is selected from imidazolyl, furyl, thienyl, isoxazolyl, thiazolyl, oxazolyl, isothiazolyl, pyrrolyl, quinolinyl, isoquinolinyl, indolyl, benzimidazolyl, benzopyranyl, benzothiophenyl, benzothiazolyl, benzooxazolyl, and wherein further preferably said mono- or bicyclic-heteroaryl is selected from isoxazolyl, thiazolyl, oxazolyl, isothiazolyl, indolyl, benzimidazolyl, benzothiazolyl, benzooxazolyl.

In a further preferred embodiment said R₂ is selected from C₅-C₁₂alkyl, C₄-C₁₀alkoxy, C₁-C₂alkylene-C₅-C₆cycloalkyl, C₁-C₂alkylene-phenyl, C₁-C₂alkylene-biphenyl, C₁-C₂alkylene-(mono- or bicyclic-heteroaryl), wherein independently in said C₁-C₂alkylene one —CH₂— moiety is optionally replaced by —CH(NH)— or —O—, and wherein said phenyl, biphenyl, C₅-C₆-cycloalkyl, and mono- or bicyclic-heteroaryl are each independently optionally substituted with one or more, typically and preferably one or two, substituents selected from methyl, ethyl, fluorine, chlorine, methoxy, wherein said mono- or bicyclic-heteroaryl is selected from imidazolyl, furyl, thienyl, isoxazolyl, thiazolyl, oxazolyl, isothiazolyl, pyrrolyl, quinolinyl, isoquinolinyl, indolyl, benzimidazolyl, benzopyranyl, benzothiophenyl, benzothiazolyl, benzooxazolyl.

In a further preferred embodiment said R₂ is selected from C₅-C₁₂alkyl, C₄-C₁₀alkoxy, C₁-C₂alkylene-C₅-C₆cycloalkyl, C₁-C₂alkylene-phenyl, C₁-C₂alkylene-biphenyl, C₁-C₂alkylene-(mono- or bicyclic-heteroaryl), wherein independently in said C₁-C₂alkylene one —CH₂— moiety is optionally replaced by —CH(NH)— or —O—, and wherein said phenyl, biphenyl, C₅-C₆-cycloalkyl, and mono- or bicyclic-heteroaryl are each independently optionally substituted with one or more, typically and preferably one or two, substituents selected from methyl, ethyl, fluorine, chlorine, methoxy, wherein said mono- or bicyclic-heteroaryl is selected from isoxazolyl, thiazolyl, oxazolyl, isothiazolyl, indolyl, benzimidazolyl, benzothiazolyl, benzooxazolyl.

In a further preferred embodiment said R₂ is selected from C₅-C₁₂alkyl, C₄-C₁₀alkoxy, C₁-C₂alkylene-C₅-C₆cycloalkyl, CH₂-phenyl, CH₂-biphenyl, CH₂—O-biphenyl, CH₂-(mono- or bicyclic-heteroaryl), wherein said phenyl, biphenyl, C₅-C₆-cycloalkyl, and mono- or bicyclic-heteroaryl are each independently optionally substituted with one or more, typically and preferably one or two, substituents selected from C₁-C₂alkyl, C₁-C₂haloalkyl, halogen, C₁-C₂alkoxy, wherein preferably said mono- or bicyclic-heteroaryl is selected from isoxazolyl, thiazolyl, oxazolyl, isothiazolyl, indolyl, benzimidazolyl, benzothiazolyl, benzooxazolyl, and wherein further preferably said mono- or bicyclic-heteroaryl is selected from, thiazolyl, indolyl, benzothiazolyl and benzooxazolyl.

In a further preferred embodiment said R₂ is selected from C₅-C₁₂alkyl, C₄-C₁₀alkoxy, C₁-C₂alkylene-C₅-C₆cycloalkyl, CH₂-phenyl, CH₂-biphenyl, CH₂—O-biphenyl, CH₂-(mono- or bicyclic-heteroaryl), wherein said phenyl, biphenyl, C₅-C₆-cycloalkyl, and mono- or bicyclic-heteroaryl are each independently optionally substituted with one or more, typically and preferably one or two, substituents selected from methyl, ethyl, fluorine, chlorine, methoxy, wherein preferably said mono- or bicyclic-heteroaryl is selected from isoxazolyl, thiazolyl, oxazolyl, isothiazolyl, indolyl, benzimidazolyl, benzothiazolyl, benzooxazolyl, and wherein further preferably said mono- or bicyclic-heteroaryl is selected from, thiazolyl, indolyl, benzothiazolyl and benzooxazolyl.

In a further preferred embodiment said R₂ is selected from C₅-C₁₂alkyl, C₄-C₁₀alkoxy, C₁-C₂alkylene-C₅-C₆cycloalkyl, CH₂-phenyl, CH₂-biphenyl, CH₂—O-biphenyl, CH₂-(mono- or bicyclic-heteroaryl), wherein said phenyl, biphenyl, C₅-C₆-cycloalkyl, and mono- or bicyclic-heteroaryl are each independently optionally substituted with one or more, typically and preferably one or two, substituents selected from methyl, ethyl, fluorine, chlorine, methoxy, wherein said mono- or bicyclic-heteroaryl is selected from isoxazolyl, thiazolyl, oxazolyl, isothiazolyl, indolyl, benzimidazolyl, benzothiazolyl, benzooxazolyl.

In a further preferred embodiment said R₂ is selected from C₅-C₁₂alkyl, C₄-C₁₀alkoxy, C₁-C₂alkylene-C₅-C₆cycloalkyl, CH₂-phenyl, CH₂-biphenyl, CH₂—O-biphenyl, CH₂-(mono- or bicyclic-heteroaryl), wherein said phenyl, biphenyl, C₅-C₆-cycloalkyl, and mono- or bicyclic-heteroaryl are each independently optionally substituted with one or more, typically and preferably one or two, substituents selected from methyl, ethyl, fluorine, chlorine, methoxy, wherein said mono- or bicyclic-heteroaryl is selected from, thiazolyl, indolyl, benzothiazolyl and benzooxazolyl.

In a further very preferred embodiment said R₂ is selected from

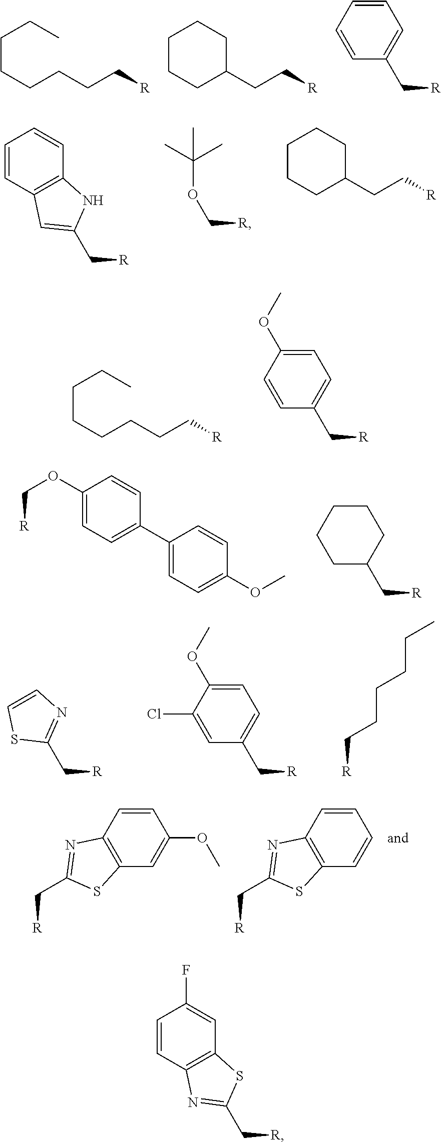

wherein R indicates the attachment to the CH-moiety depicted in formula (I).

In a further very preferred embodiment, said R₂ is selected from C₅-C₁₂alkyl, C₁-C₂alkylene-C₅-C₆cycloalkyl, CH₂-phenyl, CH₂-biphenyl and CH₂—O-biphenyl and a mono- or bicyclic-heteroaryl selected from, thiazolyl, indolyl, benzothiazolyl and benzooxazolyl, wherein said phenyl, biphenyl and mono- or bicyclic-heteroaryl is optionally substituted with one or two substituents selected from methyl, ethyl, fluorine, chlorine and methoxy.

In a further very preferred embodiment, said R₂ is selected from

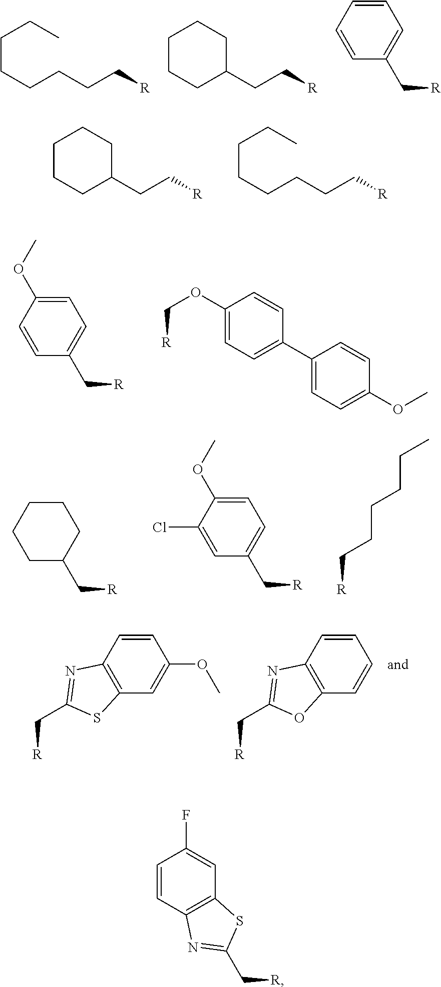

wherein R indicates the attachment to the CH-moiety depicted in formula (I).

In a further very preferred embodiment, said R₂ is selected from C₅-C₁₂alkyl, C₁-C₂alkylene-C₅-C₆cycloalkyl, CH₂-biphenyl and CH₂—O-biphenyl and a bicyclic-heteroaryl selected from benzothiazolyl and benzooxazolyl.

In a further very preferred embodiment, said R₂ is selected from

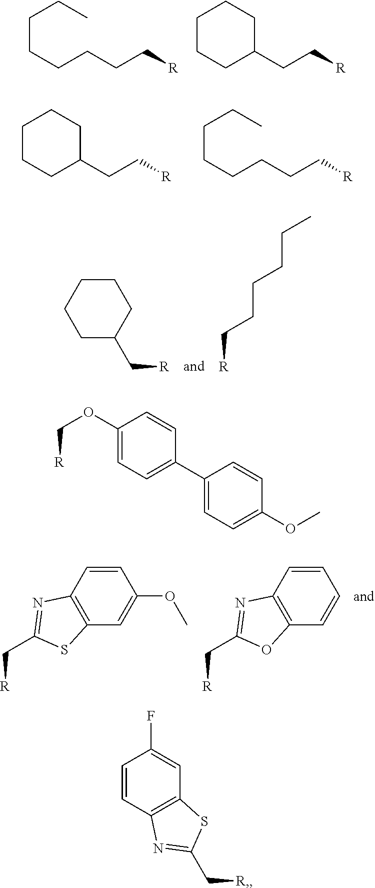

wherein R indicates the attachment to the CH-moiety depicted in formula (I).

In a further very preferred embodiment, said R₂ is

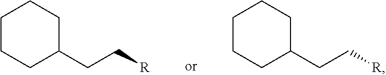

wherein R indicates the attachment to the CH-moiety depicted in formula (I).

In a further very preferred embodiment, said R₂ is

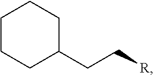

wherein R indicates the attachment to the CH-moiety depicted in formula (I).

In a further preferred embodiment, said R₃ is

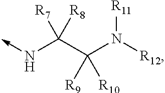

wherein
R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a carbocyclic ring, and wherein
   R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a heteroaryl or a heterocyclyl, each independently optionally and preferably substituted with halogen, C₁-C₄alkyl, OR₁₃, NR₁₄R₁₅; wherein R₁₃, R₁₄, R₁₅ are independently at each occurrence H, C₁-C₄alkyl, and wherein the arrow indicates the attachment to the C(O)-moiety depicted in formula (I).

In a further preferred embodiment, said R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a carbocyclic or heterocyclic ring, preferably a carbocyclic ring, and wherein R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a mono- or bicyclic heteroaryl or a a mono- or bicyclic heterocyclyl, preferably selected from a pyridinyl, imidazolyl, imidazopyridinyl, pyrimidinyl, pyrazolyl, triazolyl, pyrazinyl, tetrazolyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, indazolyl, indolizinyl, pyridazinyl, triazinyl, isoindolyl, purinyl, pyrrolidinyl, piperidinyl, morpholinyl, thiomorpholinyl, piperazinyl, homopiperazinyl, 2-pyrrolinyl, 3-pyrrolinyl, indolinyl, dihyrooxazolyl, pyrazolinyl, pyrazolidinyl, imidazolinyl, imidazolidinyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic or monocyclic heterocyclic ring, preferably a monocyclic carbocyclic ring, wherein further preferably said monocyclic carbocyclic ring is selected from cyclopropyl, cyclobutyl, cyclopentyl or cyclohexyl, and wherein again further preferably said monocyclic carbocyclic ring is cyclobutyl or cyclopentyl, and wherein R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a mono- or bicyclic heteroaryl or a a mono- or bicyclic heterocyclyl, preferably selected from a pyridinyl, imidazolyl, imidazopyridinyl, pyrimidinyl, pyrazolyl, triazolyl, pyrazinyl, tetrazolyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, indazolyl, indolizinyl, pyridazinyl, triazinyl, isoindolyl, purinyl, pyrrolidinyl, piperidinyl, morpholinyl, thiomorpholinyl, piperazinyl, homopiperazinyl, 2-pyrrolinyl, 3-pyrrolinyl, indolinyl, dihyrooxazolyl, pyrazolinyl, pyrazolidinyl, imidazolinyl, imidazolidinyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic or monocyclic heterocyclic ring, preferably a monocyclic carbocyclic ring, wherein further preferably said monocyclic carbocyclic ring is selected from cyclopropyl, cyclobutyl, cyclopentyl or cyclohexyl, and wherein again further preferably said monocyclic carbocyclic ring is cyclobutyl or cyclopentyl, and wherein R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heteroaryl or a monocyclic heterocyclyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic or monocyclic heterocyclic ring, preferably a monocyclic carbocyclic ring, wherein further preferably said monocyclic carbocyclic ring is selected from cyclopropyl, cyclobutyl, cyclopentyl or cyclohexyl, and wherein again further preferably said monocyclic carbocyclic ring is cyclobutyl or cyclopentyl, and wherein R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heteroaryl or a monocyclic heterocyclyl, wherein said monocyclic heteroaryl or said monocyclic heterocyclyl comprise one or two heteroatoms (including said nitrogen atom to which R₁₁ and R₁₂ are attached) selected from nitrogen, oxygen and sulphur, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic or monocyclic heterocyclic ring, preferably a monocyclic carbocyclic ring, wherein further preferably said monocyclic carbocyclic ring is selected from cyclopropyl, cyclobutyl, cyclopentyl or cyclohexyl, and wherein again further preferably said monocyclic carbocyclic ring is cyclobutyl or cyclopentyl, and wherein R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heteroaryl or a monocyclic heterocyclyl selected from a pyridinyl, imidazolyl, pyrimidinyl, pyrazolyl, triazolyl, pyrazinyl, tetrazolyl, isoxazolyl, thiazolyl, oxadiazolyl, oxazolyl, isothiazolyl, pyrrolyl, pyridazinyl, triazinyl, pyrrolidinyl, piperidinyl, morpholinyl, thiomorpholinyl, piperazinyl, 2-pyrrolinyl, 3-pyrrolinyl, dihyrooxazolyl, pyrazolinyl, pyrazolidinyl, imidazolinyl, imidazolidinyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic ring, wherein further preferably said monocyclic carbocyclic ring is selected from cyclopropyl, cyclobutyl, cyclopentyl or cyclohexyl, and wherein again further preferably said monocyclic carbocyclic ring is cyclobutyl or cyclopentyl, and wherein R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heterocyclic ring, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic or monocyclic heterocyclic ring, preferably a monocyclic carbocyclic ring, wherein further preferably said monocyclic carbocyclic ring is selected from cyclopropyl, cyclobutyl, cyclopentyl or cyclohexyl, and wherein again further preferably said monocyclic carbocyclic ring is cyclobutyl or cyclopentyl, and wherein R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heterocyclyl, wherein said monocyclic heterocyclyl comprise one or two heteroatoms (including said nitrogen atom to which R₁₁ and R₁₂ are attached) selected from nitrogen, oxygen and sulphur, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic ring, wherein further preferably said monocyclic carbocyclic ring is selected from cyclopropyl, cyclobutyl, cyclopentyl or cyclohexyl, and wherein again further preferably said monocyclic carbocyclic ring is cyclobutyl or cyclopentyl, and wherein R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heterocyclic ring selected from imidazolyl, pyrimidinyl, pyrazolyl, triazolyl, pyrazinyl, tetrazolyl, isoxazolyl, thiazolyl, oxadiazolyl, oxazolyl, isothiazolyl, pyrrolyl, pyridazinyl, triazinyl, pyrrolidinyl, piperidinyl, morpholinyl, thiomorpholinyl, piperazinyl, 2-pyrrolinyl, 3-pyrrolinyl, dihyrooxazolyl, pyrazolinyl, pyrazolidinyl, imidazolinyl, imidazolidinyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic ring selected from cyclopropyl, cyclobutyl, cyclopentyl or cyclohexyl, preferably said monocyclic carbocyclic ring is cyclobutyl or cyclopentyl, and wherein R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heterocyclic ring selected from piperidinyl, morpholinyl, thiomorpholinyl, preferably from piperidinyl or morpholinyl, and further preferably from morpholinyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further very preferred embodiment said R₃ is selected from

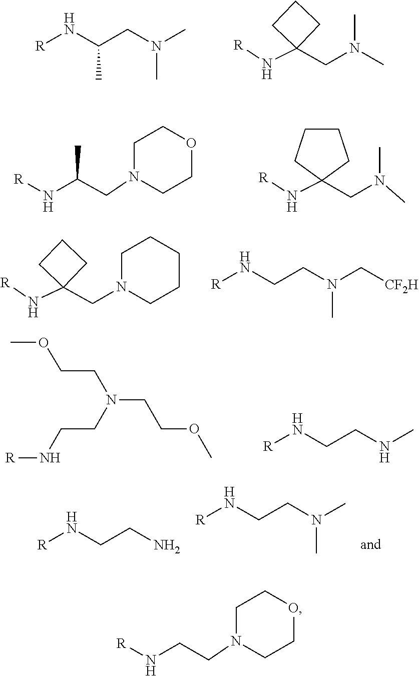

wherein R indicates the attachment to the C(O)-moiety depicted in formula (I).

In a further very preferred embodiment, said R₃ is selected from

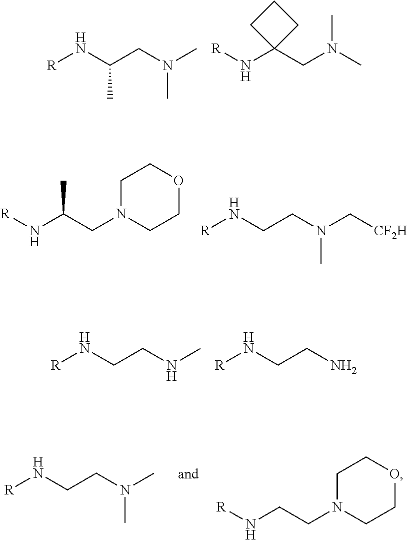

wherein R indicates the attachment to the C(O)-moiety depicted in formula (I).

In a further very preferred embodiment, said R₃ is

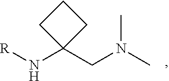

wherein R indicates the attachment to the C(O)-moiety depicted in formula (I).

In a further very preferred embodiment, said compound of formula (I) is selected from
6025: (2S)-2-[(2S)-4-cyclohexyl-2-{[(2S)-1-(2-methyl-1,3-oxazole-4-carbonyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
6027: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
6253: (2S)—N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}decanamide;
6328: (2S)—N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methyl-2-[(2S)-2-{[(2S)-1-(2-methyl-1,3-oxazole-4-carbonyl)pyrrolidin-2-yl]formamido}-3-phenylpropanamido]pentanamide;
6781: (2S)—N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}hexanamide;
7058: (2S)-2-{[(2S)-1-(2-methyl-1,3-oxazole-4-carbonyl)pyrrolidin-2-yl]formamido}-N-[(1S)-3-methyl-1-[(1-methyl-1-{[(1S)-3-methyl-1-{[(1S)-3-methyl-1-{[1-methyl-1-({1-methyl-1-[(2-{[2-(methylamino)ethyl]carbamoyl}ethyl)carbamoyl]ethyl}carbamoyl) ethyl]carbamoyl}butyl]carbamoyl}butyl]carbamoyl}ethyl)carbamoyl]butyl]decanamide;
7059: (2S)-2-[(2S)-3-cyclohexyl-2-{[(2S)-1-(2-methyl-1,3-oxazole-4-carbonyl)pyrrolidin-2-yl]formamido}propanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
7824: (2S)—N-{1-[(1-{[2-({1-[(dimethylamino)methyl]cyclobutyl}carbamoyl) ethyl]carbamoyl}-1-methylethyl)carbamoyl]-1-methylethyl}-2-[(2S)-2-{2-[(2S)-2-[(2S)-2-{[(2S)-1-(4-fluorobenzoyl)pyrrolidin-2-yl]formamido}-3-({4′-methoxy-[1,1′-biphenyl]-4-yl}oxy)propanamido]-4-methylpentanamido]-2-methylpropanamido}-4-methylpentanamido]-4-methylpentanamide;
7844: (2S)—N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-2-{[(2S)-1-(4-fluorobenzoyl)pyrrolidin-2-yl]formamido}-3-(6-methoxy-1,3-benzothiazol-2-yl)propanamido]-4-methylpentanamide;
7848: (2S)—N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-3-(6-fluoro-1,3-benzothiazol-2-yl)-2-{[(2S)-1-(4-fluorobenzoyl)pyrrolidin-2-yl]formamido}propanamido]-4-methylpentanamide;
7850: (2S)-2-[(2S)-3-(1,3-benzoxazol-2-yl)-2-{[(2S)-1-(4-fluorobenzoyl) pyrrolidin-2-yl]formamido}propanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
8341: (2S)-2-[(2S)-4-cyclohexyl-2-{[(2S)-1-(4-fluorobenzoyl)pyrrolidin-2-yl]formamido}butanamido]-4-methyl-N-(1-methyl-1-{[(1S)-3-methyl-1-{[(1S)-3-methyl-1-{[1-methyl-1-({1-methyl-1-[(2-{[2-(morpholin-4-yl)ethyl]carbamoyl}ethyl)carbamoyl]ethyl}carbamoyl)ethyl]carbamoyl}butyl]carbamoyl}butyl]carbamoyl}ethyl)pentanamide;
8342: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({2-[(2,2-difluoroethyl)(methyl)amino]ethyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
8343: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-{[1-({1-[(2-{[2-(dimethylamino)ethyl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)-4-methylpentanamide;
21164: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(2-{[2-({1-[(dimethylamino)methyl]cyclobutyl}carbamoyl)ethyl]carbamoyl}-2,2-dimethylethyl)carbamoyl]-1-methylethyl}carbamoyl)-3-methylbutyl]carbamoyl}-3-methylbutyl]carbamoyl}-1-methylethyl)-4-methylpentanamide;
21287: (2S)-2-[(2S)-4-cyclohexyl-2-{[(2S)-1-(7-methoxy-2-oxo-2H-chromene-3-carbonyl) pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
21314: (3S)-3-[(2S)-4-cyclohexyl-2-{[(2S)-1-(4-fluorobenzoyl)pyrrolidin-2-yl]formamido}butan-amido]-N-(1-{[(1S)-1-{[(1S)-1-({1-[(1-{[2-({1-[(dimethylamino) methyl]cyclobutyl}carba-moyl)ethyl]carbamoyl}-1-methylethyl)carbamoyl]-1-methyl ethyl}carbamoyl)-3-methylbutyl]carbamoyl}-3-methylbutyl]carbamoyl}-1-methylethyl)-5-methylhexanamide;
21315: (3S)-3-[(2S)-4-cyclohexyl-2-{[(2S)-1-(4-fluorobenzoyl)pyrrolidin-2-yl]formamido}butanamido]-N-(1-{[(2S)-1-{[(2S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-4-methylpentan-2-yl]carbamoyl}-4-methylpentan-2-yl]carbamoyl}-1-methylethyl)-5-methylhexanamide;
21359: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-[(1-{[(1S)-1-{[2-({1-[(dimethylamino)methyl]cyclobutyl}carbamoyl)ethyl]carbamoyl}-2,2,2-trifluoro-1-methylethyl]carbamoyl}-1-methylethyl)carbamoyl]-3-methylbutyl]carbamoyl}-3-methylbutyl]carbamoyl}-1-methylethyl)-4-methylpentanamide;
21362: (2S)-2-[(2S)-4-cyclohexyl-2-{[(2S)-1-(4-fluorobenzoyl)pyrrolidin-2-yl]formamido}butanamido]-N-[(1S)-1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-2,2,2-trifluoro-1-methylethyl]-4-methylpentanamide;
21365: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-2-methylpropan-2-yl)carbamoyl]-1-methylethyl}carbamoyl)-3-methylbutyl]carbamoyl}-3-methylbutyl]carbamoyl}-1-methylethyl)-4-methylpentanamide;
21369: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3,3,3-trifluoropropyl]carbamoyl}-3-methylbutyl]carbamoyl}-1-methylethyl)-4-methylpentanamide;
21372: (2S)-4-cyclohexyl-N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3,3,3-trifluoropropyl]carbamoyl}-3,3,3-trifluoropropyl]carbamoyl}-1-methylethyl)carbamoyl]-3,3,3-trifluoropropyl]-2-{[(2S)-1-(4-fluorobenzoyl)pyrrolidin-2-yl]formamido}butanamide; and wherein preferably said compound of formula (I) is
6025: (2S)-2-[(2S)-4-cyclohexyl-2-{[(2S)-1-(2-methyl-1,3-oxazole-4-carbonyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide; 6027: (2S)-2-[(2S)-4-cyclohexyl-2-{[(2S)-1-(4-fluorobenzoyl) pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide; 6253: (2S)—N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}decanamide; 7824: (2S)—N-{1-[(1-{[2-({1-[(dimethylamino)methyl]cyclobutyl}carbamoyl)ethyl]carbamoyl}-1-methylethyl) carbamoyl]-1-methylethyl}-2-[(2S)-2-{2-[(2S)-2-[(2S)-2-{[(2S)-1-(4-fluorobenzoyl) pyrrolidin-2-yl]formamido}-3-({4′-methoxy-[1,1′-biphenyl]-4-yl}oxy)propanamido]-4-methylpentanamido]-2-methylpropanamido}-4-methylpentanamido]-4-methylpentanamide;
7848: (2S)—N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-3-(6-fluoro-1,3-benzothiazol-2-yl)-2-{[(2S)-1-(4-fluorobenzoyl)pyrrolidin-2-yl]formamido}propanamido]-4-methylpentanamide; 8343: (2S)-2-[(2S)-4-cyclohexyl-2-{[(2S)-1-(4-fluorobenzoyl)pyro-lidin-2-yl]formamido}butanamido]-N-(1-{[(1S)-1-{[(1S)-1-{[1-({1-[(2-{[2-(dimethylamino) ethyl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)-4-methylpentanamide;
21314: (3S)-3-[(2S)-4-cyclohexyl-2-{[(2S)-1-(4-fluorobenzoyl)pyrrolidin-2-yl]formamido}butan-amido]-N-(1-{[(1S)-1-{[(1S)-1-({1-[(1-{[2-({1-[(dimethylamino) methyl]cyclobutyl}carba-moyl)ethyl]carbamoyl}-1-methylethyl)carbamoyl]-1-methyl ethyl}carbamoyl)-3-methylbutyl]carbamoyl}-3-methylbutyl]carbamoyl}-1-methylethyl)-5-methylhexanamide; 21359: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-[(1-{[(1S)-1-{[2-({1-[(dimethylamino)methyl]cyclobutyl}carbamoyl)ethyl]carbamoyl}-2,2,2-trifluoro-1-methylethyl]carbamoyl}-1-methylethyl)carbamoyl]-3-methylbutyl]carbamoyl}-3-methylbutyl]carbamoyl}-1-methylethyl)-4-methylpentanamide; or 21369: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3,3,3-trifluoropropyl]carbamoyl}-3-methylbutyl]carbamoyl}-1-methylethyl)-4-methylpentanamide.

In a further very preferred embodiment, said compound of formula (I) is 6027: (2S)-2-[(2S)-4-cyclohexyl-2-{[(2S)-1-(4-fluorobenzoyl) pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide.

In a further very preferred embodiment, said compound of formula (I) is 6253: (2S)—N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methyl butyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}decanamide.

In a further very preferred embodiment, said compound of formula (I) is 7848: (2S)—N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-3-(6-fluoro-1,3-benzothiazol-2-yl)-2-{[(2S)-1-(4-fluorobenzoyl)pyrrolidin-2-yl]formamido}propanamido]-4-methylpentanamide.

In a further very preferred embodiment, said compound of formula (I) is 8343: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-{[1-({1-[(2-{[2-(dimethylamino)ethyl]carbamoyl}ethyl)carbamoyl]-1-methylethyl}carbamoyl)-1-methylethyl]carbamoyl}-3-methylbutyl]carbamoyl}-3-methyl butyl]carbamoyl}-1-methylethyl)-4-methylpentanamide.

In a further very preferred embodiment, said compound of formula (I) is 21369: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3,3,3-trifluoropropyl]carbamoyl}-3-methylbutyl]carbamoyl}-1-methylethyl)-4-methylpentanamide.

In a further very preferred embodiment, said compound is selected from a formula as depicted in claim 11 and selected from 6025, 6027, 6253, 6328, 6781, 7058, 7059, 7824, 7844, 7848, 7850, 8341, 8342, 8343, 21164, 21287, 21314, 21315, 21359, 21362, 21365, 21369, or 21372, and wherein preferably said compound of formula (I) is selected from a formula as depicted in claim 11 and selected from 6025, 6027, 6253, 7824, 7848, 8343, 21314, 21359, or 21369. In a further very preferred embodiment, said compound of formula (I) is formula 6027 as depicted in claim 11. In a further very preferred embodiment, said compound of formula (I) is formula 6253 as depicted in claim 11. In a further very preferred embodiment, said compound of formula (I) is formula 7848 as depicted in claim 11. In a further very preferred embodiment, said compound of formula (I) is formula 8343 as depicted in claim 11. In a further very preferred embodiment, said compound of formula (I) is formula 21369 as depicted in claim 11.

In a further aspect, the present invention provides for a compound of formula (I), or a pharmaceutically acceptable salt thereof, wherein said compound is selected from

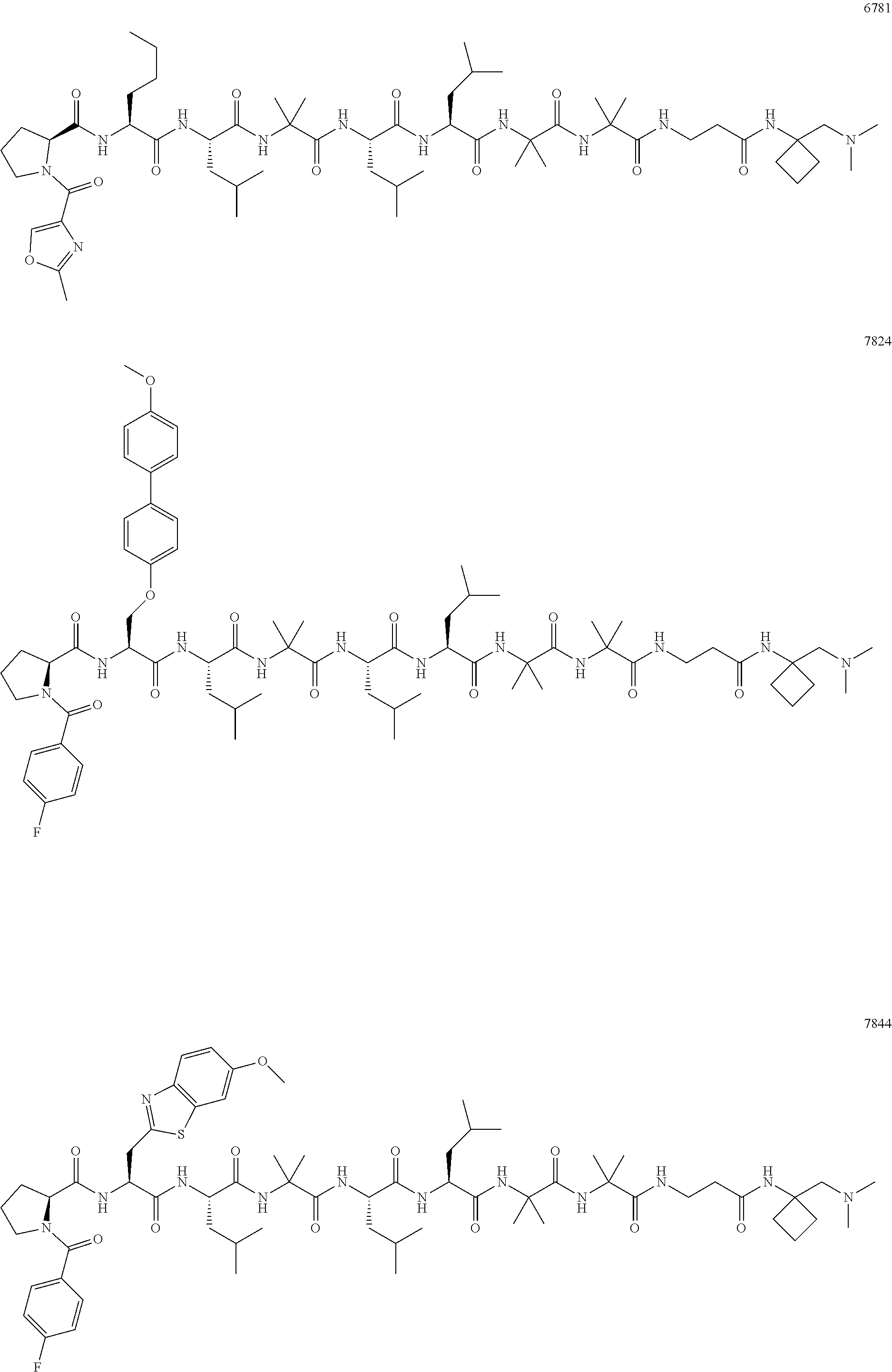

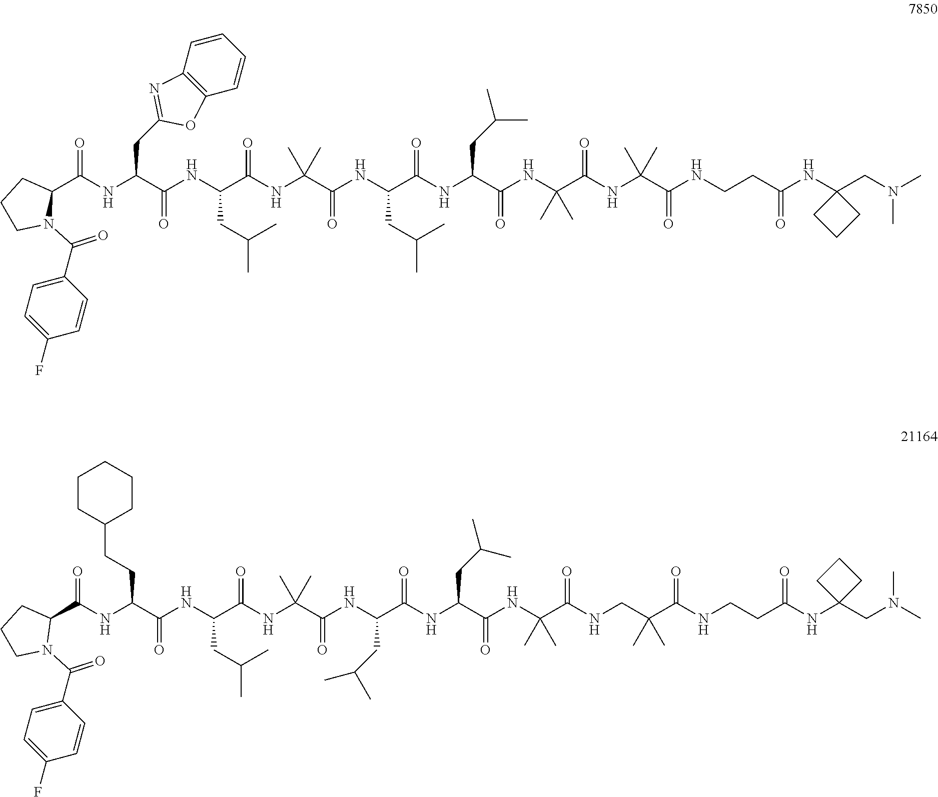 [SMILES: CC(C)C[C@H](NC(=O)[C@H](Cc1nc2ccccc2o1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC1(CN(C)C)CCC1 ; CC(C)C[C@H](NC(=O)[C@H](CC(C)C)NC(=O)C(C)(C)NC(=O)[C@H](CC(C)C)NC(=O)[C@H](CCC1CCCCC1)NC(=O)[C@@H]1CCCN1C(=O)c1ccc(F)cc1)C(=O)NC(C)(C)C(=O)NCC(C)(C)C(=O)NCCC(=O)NC1(CN(C)C)CCC1]

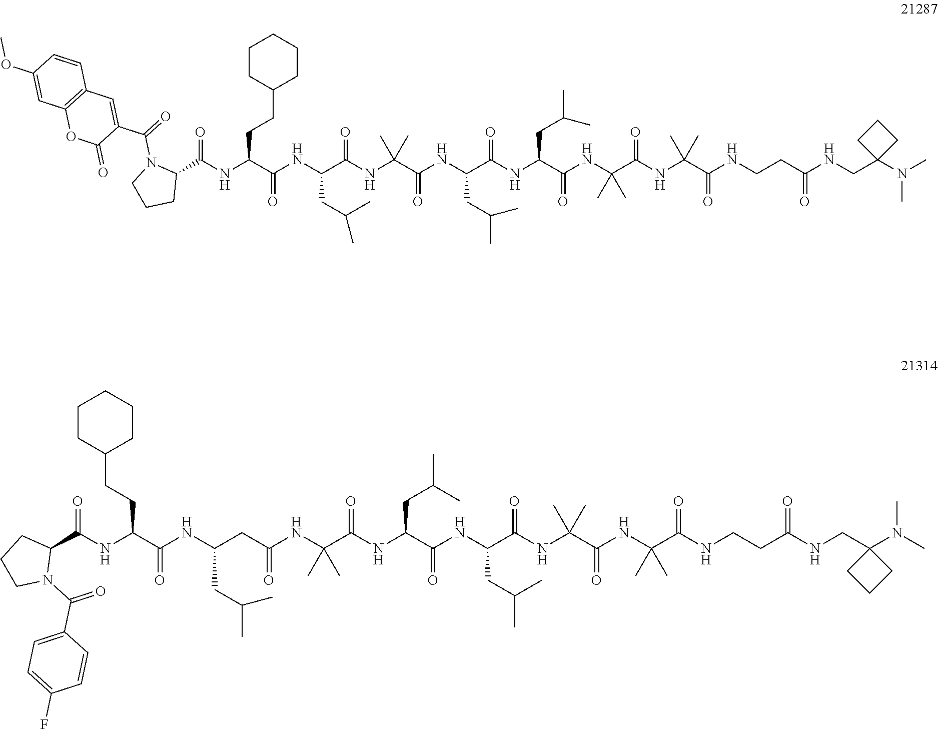

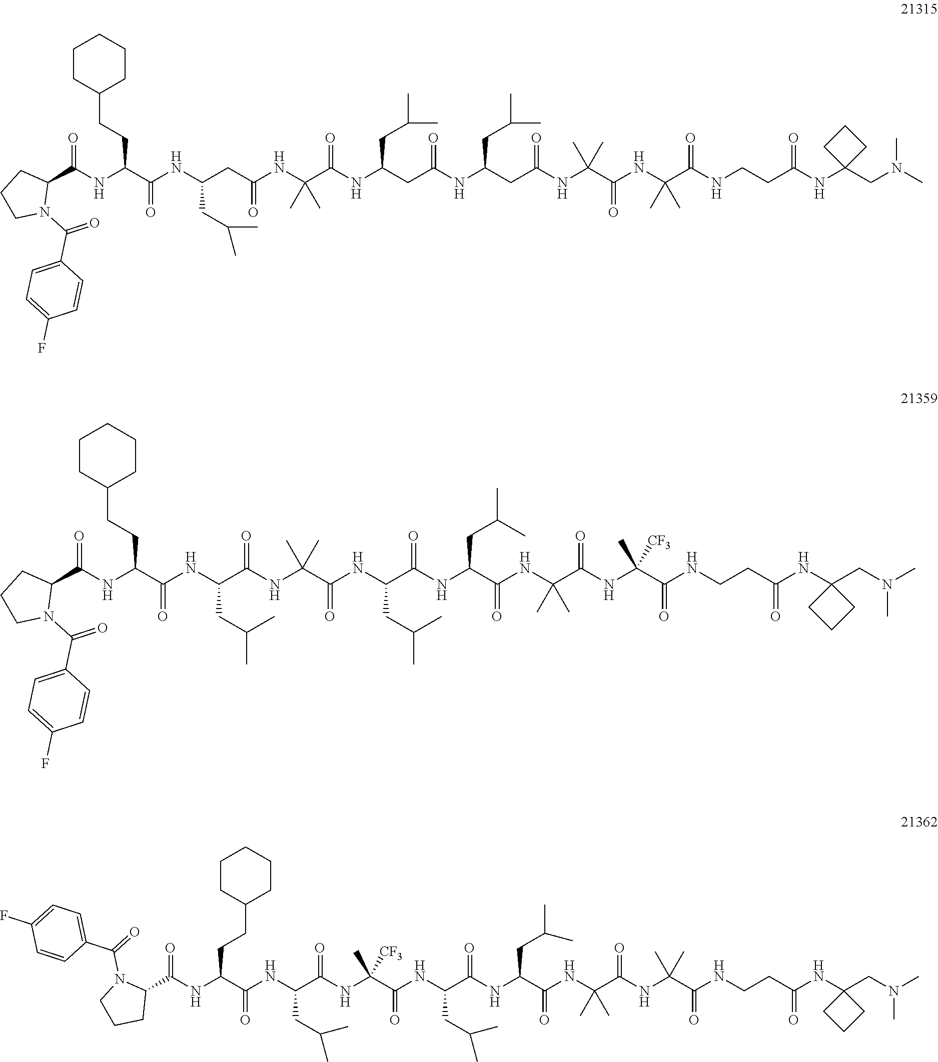

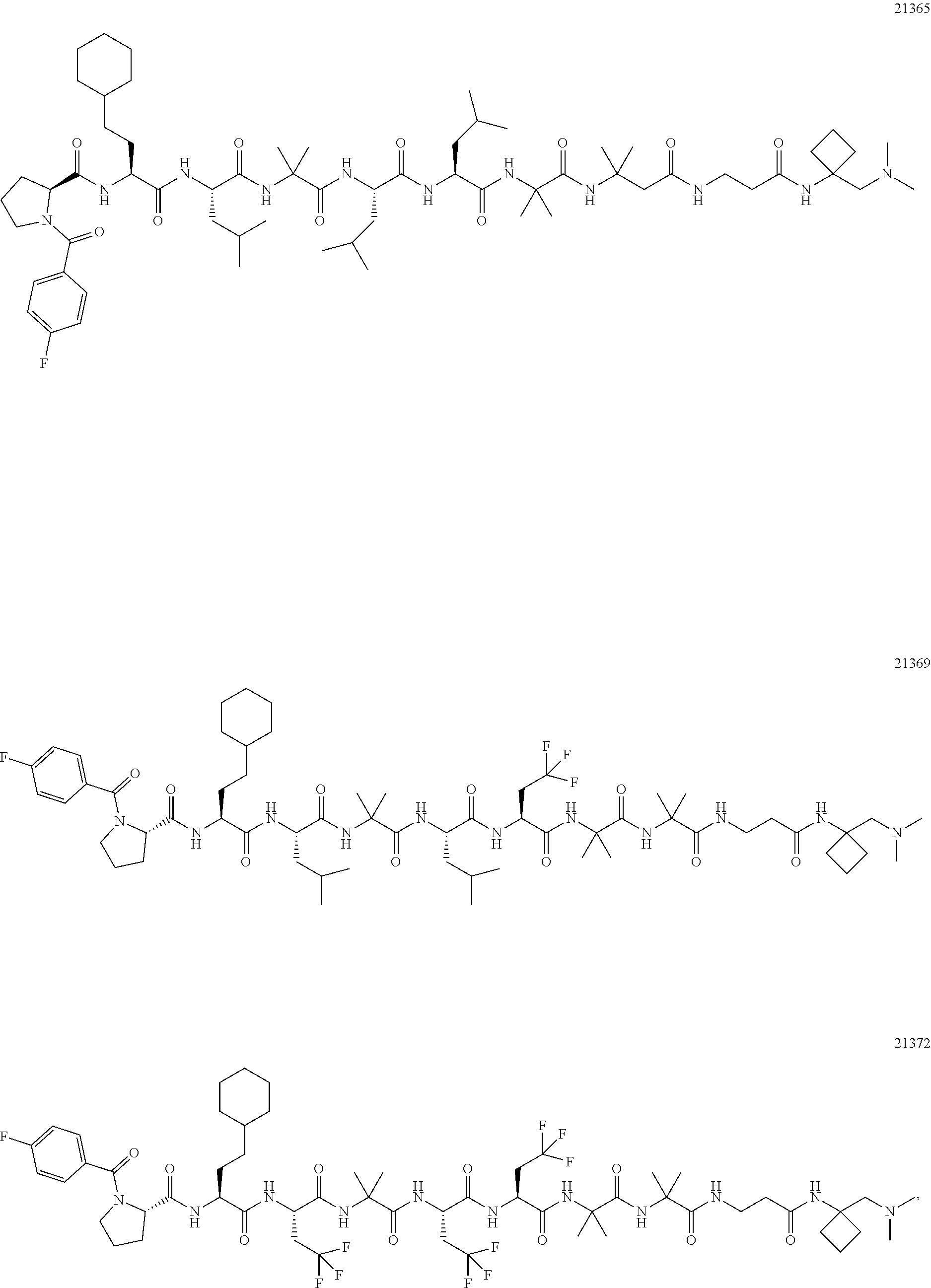

wherein preferably a compound of formula (I) is a compound selected from

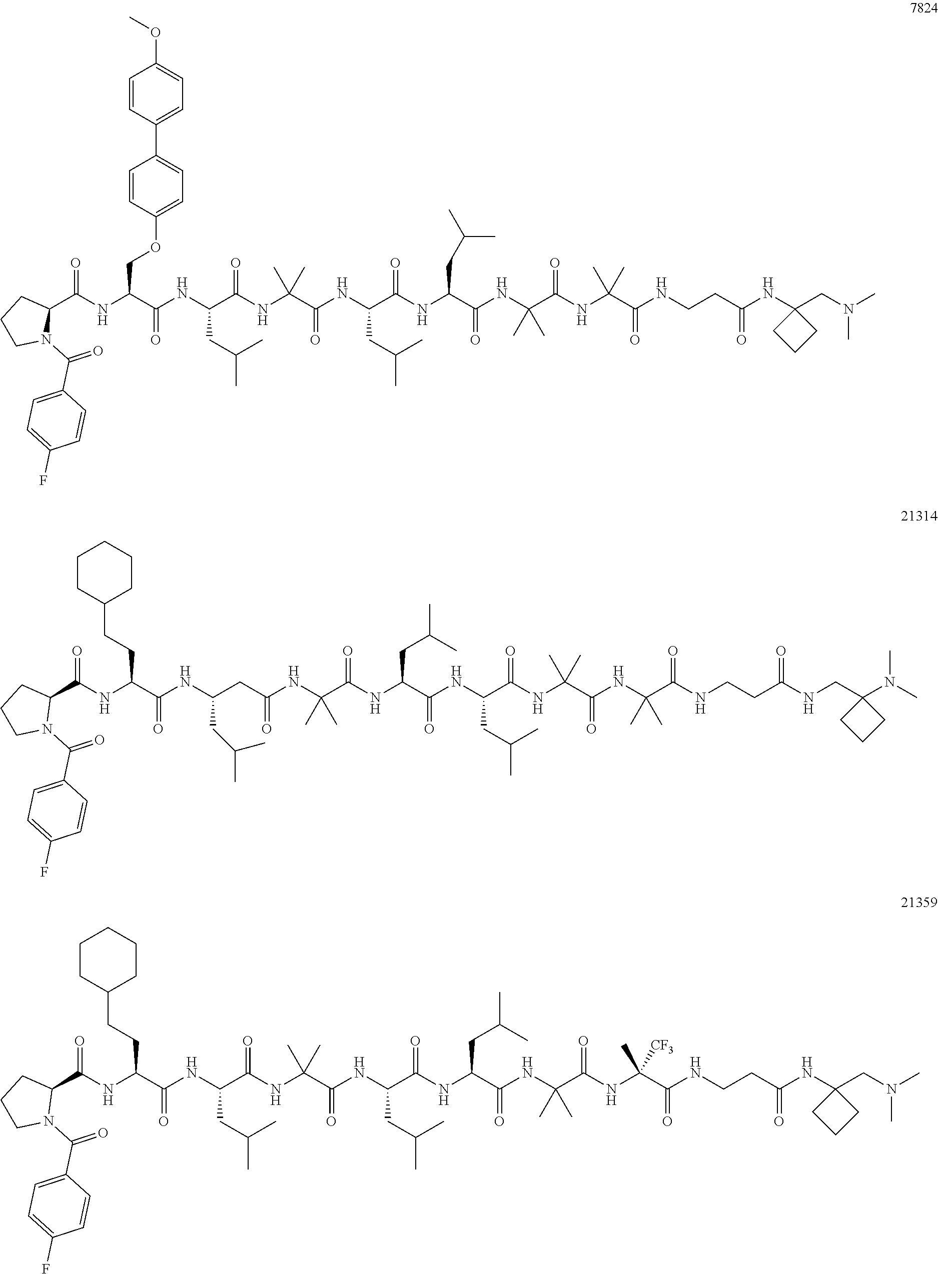

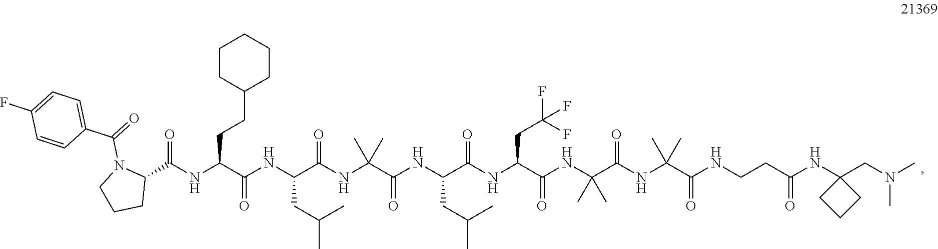 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(F)(F)F)C(=O)NC(C)(C)C(=O)NC(C)(C)C(=O)NCCC(=O)NC1(CN(C)C)CCC1]

wherein further preferably said compound is 21369: (2S)-2-[(2S)-4-cyclohexyl-2-{[(2S)-1-(4-fluorobenzoyl)pyrrolidin-2-yl]formamido}butan-amido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carba-moyl]-1-methylethyl}carbamoyl)-3,3,3-trifluoropropyl]carbamoyl}-3-methylbutyl]carba-moyl}-1-methylethyl)-4-methylpentanamide.

In a further aspect, the present invention provides for a compound of formula (I), wherein said compound is selected from
6781: (2S)—N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}hexanamide;
7824: (2S)—N-{1-[(1-{[2-({1-[(dimethylamino)methyl]cyclobutyl}carbamoyl) ethyl]carbamoyl}-1-methylethyl)carbamoyl]-1-methylethyl}-2-[(2S)-2-{2-[(2S)-2-[(2S)-2-{[(2S)-1-(4-fluorobenzoyl)pyrrolidin-2-yl]formamido}-3-({4′-methoxy-[1,1′-biphenyl]-4-yl}oxy)propanamido]-4-methylpentanamido]-2-methylpropanamido}-4-methylpentanamido]-4-methylpentanamide;
7844: (2S)—N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-2-{[(2S)-1-(4-fluorobenzoyl)pyrrolidin-2-yl]formamido}-3-(6-methoxy-1,3-benzothiazol-2-yl)propanamido]-4-methylpentanamide;
7850:(2S)-2-[(2S)-3-(1,3-benzoxazol-2-yl)-2-{[(2S)-1-(4-fluorobenzoyl) pyrrolidin-2-yl]formamido}propanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
21164: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(2-{[2-({1-[(dimethylamino)methyl]cyclobutyl}carbamoyl)ethyl]carbamoyl}-2,2-dimethylethyl)carbamoyl]-1-methylethyl}carbamoyl)-3-methylbutyl]carbamoyl}-3-methylbutyl]carbamoyl}-1-methylethyl)-4-methylpentanamide;
21287: (2S)-2-[(2S)-4-cyclohexyl-2-{[(2S)-1-(7-methoxy-2-oxo-2H-chromene-3-carbonyl) pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
21314: (3S)-3-[(2S)-4-cyclohexyl-2-{[(2S)-1-(4-fluorobenzoyl)pyrrolidin-2-yl]formamido}butan-amido]-N-(1-{[(1S)-1-{[(1S)-1-({1-[(1-{[2-({1-[(dimethylamino) methyl]cyclobutyl}carba-moyl)ethyl]carbamoyl}-1-methylethyl)carbamoyl]-1-methyl ethyl}carbamoyl)-3-methylbutyl]carbamoyl}-3-methylbutyl]carbamoyl}-1-methylethyl)-5-methylhexanamide;
21315: (3S)-3-[(2S)-4-cyclohexyl-2-{[(2S)-1-(4-fluorobenzoyl)pyrrolidin-2-yl]formamido}butanamido]-N-(1-{[(2S)-1-{[(2S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-4-methylpentan-2-yl]carbamoyl}-4-methylpentan-2-yl]carbamoyl}-1-methylethyl)-5-methylhexanamide;
21359: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-[(1-{[(1S)-1-{[2-({1-[(dimethylamino)methyl]cyclobutyl}carbamoyl)ethyl]carbamoyl}-2,2,2-trifluoro-1-methylethyl]carbamoyl}-1-methylethyl)carbamoyl]-3-methylbutyl]carbamoyl}-3-methylbutyl]carbamoyl}-1-methylethyl)-4-methylpentanamide;
21362: (2S)-2-[(2S)-4-cyclohexyl-2-{[(2S)-1-(4-fluorobenzoyl)pyrrolidin-2-yl]formamido}butanamido]-N-[(1S)-1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-2,2,2-trifluoro-1-methylethyl]-4-methylpentanamide;
21365: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-2-methylpropan-2-yl)carbamoyl]-1-methylethyl}carbamoyl)-3-methylbutyl]carbamoyl}-3-methylbutyl]carbamoyl}-1-methylethyl)-4-methylpentanamide;
21369: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3,3,3-trifluoropropyl]carbamoyl}-3-methylbutyl]carbamoyl}-1-methylethyl)-4-methylpentanamide;
21372: (2S)-4-cyclohexyl-N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3,3,3-trifluoropropyl]carbamoyl}-3,3,3-trifluoropropyl]carbamoyl}-1-methylethyl)carbamoyl]-3,3,3-trifluoropropyl]-2-{[(2S)-1-(4-fluorobenzoyl)pyrrolidin-2-yl]formamido}butanamide wherein preferably said compound is selected from
7824: (2S)—N-{1-[(1-{[2-({1-[(dimethylamino)methyl]cyclobutyl}carbamoyl) ethyl]carbamoyl}-1-methylethyl)carbamoyl]-1-methylethyl}-2-[(2S)-2-{2-[(2S)-2-[(2S)-2-{[(2S)-1-(4-fluorobenzoyl)pyrrolidin-2-yl]formamido}-3-({4′-methoxy-[1,1′-biphenyl]-4-yl}oxy)propanamido]-4-methylpentanamido]-2-methylpropanamido}-4-methylpentanamido]-4-methylpentanamide;
21314: (3S)-3-[(2S)-4-cyclohexyl-2-{[(2S)-1-(4-fluorobenzoyl)pyrrolidin-2-yl]formamido}butan-amido]-N-(1-{[(1S)-1-{[(1S)-1-({1-[(1-{[2-({1-[(dimethylamino) methyl]cyclobutyl}carba-moyl)ethyl]carbamoyl}-1-methylethyl)carbamoyl]-1-methyl ethyl}carbamoyl)-3-methylbutyl]carbamoyl}-3-methylbutyl]carbamoyl}-1-methylethyl)-5-methylhexanamide;
21359: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-[(1-{[(1S)-1-{[2-({1-[(dimethylamino)methyl]cyclobutyl}carbamoyl)ethyl]carbamoyl}-2,2,2-trifluoro-1-methylethyl]carbamoyl}-1-methylethyl)carbamoyl]-3-methylbutyl]carbamoyl}-3-methylbutyl]carbamoyl}-1-methylethyl)-4-methylpentanamide; and
21369: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3,3,3-trifluoropropyl]carbamoyl}-3-methylbutyl]carbamoyl}-1-methylethyl)-4-methylpentanamide; and
   wherein further preferably said compound is 21369: (2S)-2-[(2S)-4-cyclohexyl-2-{[(2S)-1-(4-fluorobenzoyl)pyrrolidin-2-yl]formamido}butan-amido]-N-(1-{[(1S)-1-{[(1S)-1-({1-[(1-{[2-({1-[(dimethylamino)methyl]cyclobutyl}carbamoyl)ethyl]carbamoyl}-1-methylethyl) carbamoyl]-1-methylethyl}carbamoyl)-3,3,3-trifluoropropyl]carbamoyl}-3-methylbutyl]carbamoyl}-1-methylethyl)-4-methylpentanamide.

The present invention provides a compound of formula (I) for use in a method of topical treatment of a skin cancer of a mammal, wherein said method comprises topical administration of an effective amount of said compound, or a pharmaceutically acceptable salt thereof, to said mammal. In a preferred embodiment, said mammal is a human.

In a further preferred embodiment, said topical administration is applying said compound, or a pharmaceutically acceptable salt thereof, to a skin of a mammal. In a preferred embodiment, said topical administration is applying said compound, or a pharmaceutically acceptable salt thereof, to a skin of a human. In a further preferred embodiment, said topical administration is applying said compound, or a pharmaceutically acceptable salt thereof, directly to a skin of a mammal. In a preferred embodiment, said topical administration is applying said compound, or a pharmaceutically acceptable salt thereof, directly to a skin of a human.

In a further preferred embodiment, said skin cancer is selected from a melanoma skin cancer, a non-melanoma skin cancer and a cutaneous lymphoma, and any pre-invasive form thereof. In a further preferred embodiment, said skin cancer is selected from a melanoma, a non-melanoma skin cancer, and any pre-invasive form thereof, wherein preferably said non-melanoma skin cancer is a basal cell carcinoma, Merkel cell carcinoma or a cutaneous squamous cell carcinoma, and wherein further preferably said non-melanoma skin cancer is a basal cell carcinoma or a cutaneous squamous cell carcinoma, or any precancer form thereof. In a further preferred embodiment, said skin cancer is selected from a melanoma skin cancer, a non-melanoma skin cancer and a cutaneous lymphoma, and any pre-invasive form thereof, wherein preferably said non-melanoma skin cancer is a basal cell carcinoma, Merkel cell carcinoma or a cutaneous squamous cell carcinoma, and wherein further preferably said non-melanoma skin cancer is a basal cell carcinoma or a cutaneous squamous cell carcinoma, or any pre-invasive form thereof. In a further preferred embodiment, said skin cancer is selected from a melanoma, a non-melanoma skin cancer, and any pre-invasive form thereof, wherein preferably said non-melanoma skin cancer is a basal cell carcinoma, Merkel cell carcinoma or a cutaneous squamous cell carcinoma, and wherein further preferably said non-melanoma skin cancer is a basal cell carcinoma or a cutaneous squamous cell carcinoma, or any pre-invasive form thereof.

In a further preferred embodiment, said skin cancer is selected from a melanoma and a non-melanoma skin cancer, wherein preferably said non-melanoma skin cancer is a basal cell carcinoma or a cutaneous squamous cell carcinoma. In a further preferred embodiment, said skin cancer is a melanoma. In a further preferred embodiment, said skin cancer is a non-melanoma skin cancer, wherein preferably said non-melanoma skin cancer is a basal cell carcinoma, Merkel cell carcinoma or a cutaneous squamous cell carcinoma, or any pre-invasive form thereof, and wherein further preferably said non-melanoma skin cancer is a basal cell carcinoma or a cutaneous squamous cell carcinoma, or any pre-invasive form thereof. In a further preferred embodiment, said skin cancer is a basal cell carcinoma. In a further preferred embodiment, said skin cancer is a cutaneous squamous cell carcinoma. In a preferred embodiment, said skin cancer is a pre-invasive form of non-melanoma skin cancer. In another preferred embodiment, said skin cancer is a non-melanoma skin cancer. In another preferred embodiment, said skin cancer is a cutaneous lymphoma.

In a further preferred embodiment, said cutaneous squamous cell carcinoma (cSCC) is an invasive cSCC. In a further preferred embodiment, said cutaneous squamous cell carcinoma (cSCC) is a metastatic cSCC. In a further preferred embodiment, said basal cell carcinoma is selected from the group consisting of superficial basal cell carcinoma (also known as “in situ basal cell carcinoma” or “superficial multicentric basal-cell carcinoma”), infiltrative basal cell carcinoma and nodular basal cell carcinoma. In a preferred embodiment, said basal cell carcinoma is a superficial basal cell carcinoma (also known as “in situ basal cell carcinoma” or “superficial multicentric basal-cell carcinoma”). In a further preferred embodiment, said basal cell carcinoma is an infiltrative basal cell carcinoma. In a further preferred embodiment, said basal cell carcinoma is a nodular basal cell carcinoma. In another embodiment, said basal cell carcinoma is selected from the group consisting of cystic basal cell carcinoma, cicatricial basal cell carcinoma (also known as “morpheaform basal cell carcinoma” or “morphoeic basal cell carcinoma”), micronodular basal cell carcinoma, pigmented basal cell carcinoma, rodent ulcer (also known as “Jacob's ulcer”), fibroepithelioma of Pinkus, polypoid basal cell carcinoma, pore-like basal cell carcinoma and aberrant basal cell carcinoma.

In another preferred embodiment, said skin cancer is a pre-invasive form of a non-melanoma skin cancer, wherein said pre-invasive form is selected from the group consisting of cutaneous squamous cell carcinoma in situ (cSCCis, also known as “Bowen's disease”) and precancerous actinic keratosis (AK). In another preferred embodiment, said skin cancer is a pre-invasive form of a non-melanoma skin cancer, wherein said pre-invasive form is cutaneous squamous cell carcinoma in situ (cSCCis, also known as “Bowen's disease”). In a further preferred embodiment, said skin cancer is a pre-invasive form of a non-melanoma skin cancer, wherein said pre-invasive form is precancerous actinic keratosis (AK). In another preferred embodiment, said skin cancer is actinic keratosis (AK). In another preferred embodiment, said skin cancer is cutaneous squamous cell carcinoma in situ (cSCCis). In another preferred embodiment, said skin cancer is a cutaneous lymphoma, wherein preferably said cutaneous lymphoma is a cutaneous T-cell lymphoma (CTCL) or a cutaneous B-cell lymphoma (CBCL). In another preferred embodiment, said skin cancer is a cutaneous lymphoma, wherein said cutaneous lymphoma is a cutaneous T-cell lymphoma (CTCL). In another preferred embodiment, said skin cancer is a cutaneous lymphoma, wherein said cutaneous lymphoma is a cutaneous B-cell lymphoma (CBCL). In another preferred embodiment, said skin cancer is a pre-invasive form of a non-melanoma skin cancer, and wherein said pre-invasive form is actinic keratosis (AK). In another preferred embodiment, said skin cancer is a pre-invasive form of a non-melanoma skin cancer, and said pre-invasive form is cSCC in situ (cSCCis).

The compounds and methods described herein are advantageously used to inhibit proliferation of skin cancer cells. Preferably, the compounds and methods provide topical treatment of a mammal, preferably a human, against skin cancer. The methods provides topical treatment against the various types of skin cancer, in particular of basal cell carcinoma, cutaneous squamous cell carcinoma or melanoma or cutaneous lymphoma. Thus, topical administering of a compound of formula (I) or a pharmaceutically acceptable salt thereof, or a pharmaceutical composition comprising the same provide treatment of a patient against skin cancer.

### EXAMPLES

All reagents and solvents used in the synthesis were purchased from Sigma Aldrich, Bachem, Iris Biotech, Fluorochem, Enamine, Combi-Blocks and used as received. Solvents were stored over molecular sieves 4 Å. Peptides were synthesized on a 0.25-mmol or 0.10-mmol scale on Fmoc-3-Ala Wang resin (0.70 mmol g⁻¹) using a Liberty Blue microwave peptide synthesizer (CEM Corp., Matthews, NC) employing Fmoc solid-phase techniques with repeated steps of coupling, deprotection, and washing. Coupling was performed as follows: Fmoc-L-amino acids or the capping group (5.0 equiv., 0.2 M in DMF), DIC (5.0 equiv., 0.5 M in DMF), and Oxyma (5.0 equiv., 1.0 M in DMF) for 4 minutes with microwave irradiation at 90° C. For the second AIB coupling, a double coupling with each 15 minutes microwave irradiation at 90° C. was performed. Fmoc deprotection was performed as follows: 10% piperazine in NMP/ethanol (9:1) or 20% piperidine in DMF for 1 minute with microwave irradiation at 90° C. Following synthesis, the peptide was cleaved from the resin by treatment with a cleavage mixture (1 mL/0.1 g resin) consisting of TFA/H₂O (95:5) for 90 minutes at ambient temperature. The suspended resin was removed by filtration and concentrated in vacuo. The crude peptides were dissolved in acetonitrile and were purified by ISCO chromatography system using as mobile phase H₂O/acetonitrile (95:5, v/v) and acetonitrile/H₂O, gradient ACN 10% to 100%, to give peptide acids ≥90% pure. The purified peptide acid (1.0 equiv.) was coupled in solution with suitable primary or secondary amines (2.0 equiv.) in presence of DIPEA (3.0 equiv.) and COMU (2.0 equiv.) or HATU to give the final peptide. Solvent was partially removed, and the crude peptide was directly purified by Gilson PLC 2020 Personal Purification System, as described above, to give final products ≥95% pure.

High-resolution mass spectrometry was performed on an Agilent Technologies 6530 Q-TOF. Mass and purity of final compounds was determined by UPLC-MS using a Waters Acquity system equipped with a Waters BEH C18 column and diode array detector (254 nm). The mobile phase consisted of H₂O-Acetonitrile (solvent A, 97:3 v/v, LC-MS Ultra Chromasolv®, UHPLC grade, Sigma-Aldrich, Germany) and 0.1% Formic acid (LC-MS grade, Sigma-Aldrich, Germany) and Acetonitrile-H₂O (solvent B, 97:3 v/v, LC-MS Ultra Chromasolv®, UHPLC grade, Sigma-Aldrich, Germany) and 0.1% Formic acid (LC-MS grade, Sigma-Aldrich, Germany) with a 4 min run time, 0.6 ml/min flow rate, 10 μL injection volume and a gradient elution according to the following program: linear increment starting with 100% A to 100% Bin 3 min and returning to the initial conditions within the next 1 min. MS detection of analytes was performed on a Waters Xevo® triple quadrupole mass spectrometer equipped with electrospray ionization (ESI) interface (Waters, Xevo® TQD QCA065) in positive and negative ion mode with mass range from 100-1500 m/z.

### Example 1

### General Procedure for the Synthesis for Compounds of Formula (I)

The peptides were synthesized using a Liberty Blue microwave peptide synthesizer (CEM Corp., Matthews, NC) starting from Fmoc-beta-Ala linked to Wang resin as depicted in Scheme 1, employing Fmoc solid-phase techniques with repeated steps of coupling, deprotection, and washing. Coupling was performed as follows: Fmoc-L-amino acids or the capping group (5.0 equiv., 0.2 M in DMF), DIC (5.0 equiv., 0.5 M in DMF), and Oxyma (5.0 equiv., 1.0 M in DMF) for 4 minutes with microwave irradiation at 90° C. For the second Aib coupling, a double coupling with each 15 minutes microwave irradiation at 90° C. was performed. Fmoc deprotection was performed as follows: 10% piperazine in NMP/ethanol (9:1) or 20% piperidine in DMF for 1 minute with microwave irradiation at 90° C. By employing sequentially the building blocks listed in Table 1 (Amino acids used in steps 1 to 8, Scheme 1) and free acid derivatives (step 9, Scheme 1) listed in Table 2, the different peptides were assembled on solid phase support in typically and preferably 9 steps. Following synthesis, the peptide was cleaved from the resin by treatment with a cleavage mixture (1 mL/0.1 g resin) consisting of TFA/H₂O (95:5) for 90 minutes at ambient temperature. The suspended resin was removed by filtration and concentrated in vacuo. The crude peptides were dissolved in acetonitrile and were purified by ISCO chromatography system. The purified peptide acid was coupled in solution with suitable primary or secondary amines reported in Table 3 (step 10, Scheme 1) in presence of DIPEA and COMU or HATU to give the final peptide, which was then directly purified on Gilson PLC 2020 Personal Purification System.

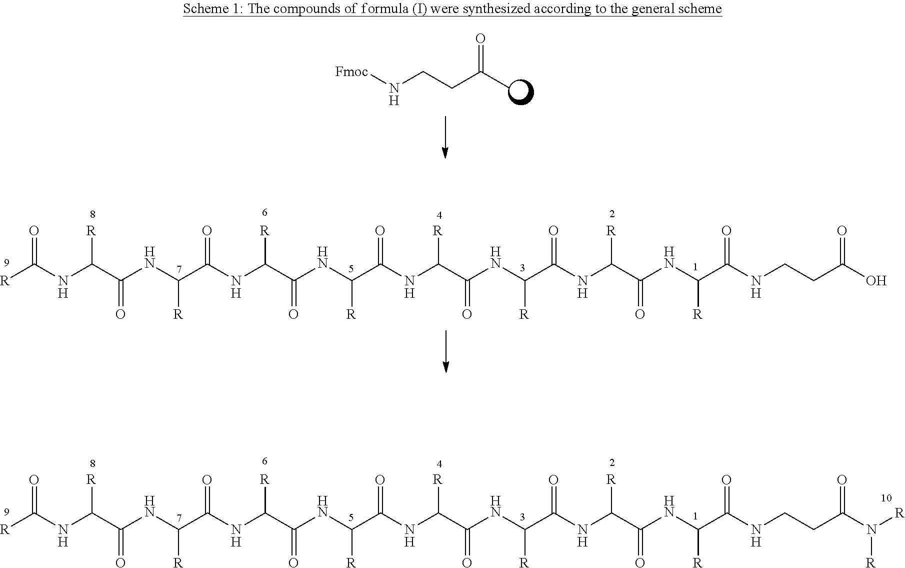

TABLE 1
Amino acid building blocks used for the synthesis of compounds of formula (I)
AMINO ACIDS

| | Structure | IUPAC Name |
|---|---|---|
| Fmoc-Aib-OH | 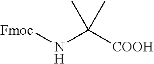 | 2-((((9H-fluoren-9-yl)methoxy) carbonyl)amino)-2- methylpropanoic acid |
| Fmoc-3-amino-3-methyl- butyric acid | 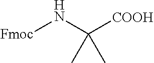 | 3-(9H-fluoren-9- ylmethoxycarbonylamino)-3- methylbutanoic acid |
| Fmoc-3-amino-2,2- dimethyl-propionic acid | 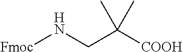 | 3-((((9H-Fluoren-9- yl)methoxy)carbonyl)amino)- 2,2-dimethylpropanoic acid |
| Fmoc-alpha-Tfm-L-Ala- OH | 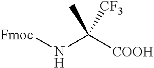 | (2S)-2-({[(9H-fluoren-9- yl)methoxy]carbonyl}amino)- 3,3,3-trifluoro-2- methylpropanoic acid |
| Fmoc-Leu-OH | 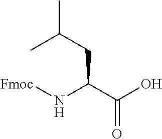 | (((9H-fluoren-9-yl)methoxy) carbonyl)-L-leucine |
| Fmoc-L-TfAbu-OH | 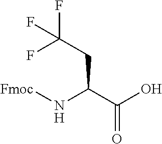 | (2S)-2-({[(9H-fluoren-9- yl)methoxy]carbonyl}amino)- 4,4,4-trifluorobutanoic acid |
| Fmoc-β-Homoleu-OH | 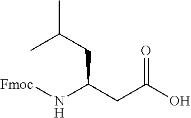 | (3S)-3-({[(9H-fluoren-9- yl)methoxy]carbonyl}amino)-5- methylhexanoic acid |
| Fmoc-(S)-2-amino- decanoic acid | 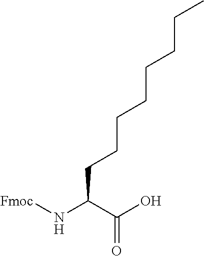 | (S)-2-((((9H-fluoren-9-yl) methoxy)carbonyl)amino) decanoic acid |
| Fmoc-Nle-OH | 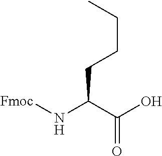 | (S)-2-((((9H-fluoren-9-yl) methoxy)carbonyl)amino) hexanoic acid |
| Fmoc-Pro-OH | 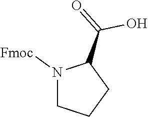 | (((9H-fluoren-9-yl)methoxy) carbonyl)-L-proline |
| Fmoc-L- homocyclohexylalanine | 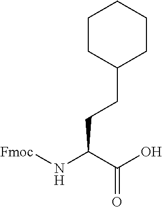 | (S)-2-((((9H-fluoren-9-yl) methoxy)carbonyl)amino)-4- cyclohexylbutanoic acid |
| Fmoc-Phe-OH | 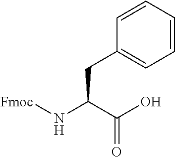 | (2S)-2-(9H-fluoren-9-yl methoxycarbonylamino)-3- phenyl-propanoic acid |
| Fmoc-Cha-OH | 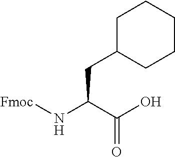 | (S)-2-((((9H-Fluoren-9-yl) methoxy)carbonyl)amino)-3- cyclohexylpropanoic acid |
| (2S)-2-({[(9H-fluoren-9- yl)methoxy]carbonyl} amino)-3-(6-fluoro-1,3- benzothiazol-2-yl) propanoic acid | 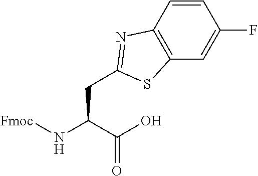 | (2S)-2-({[(9H-fluoren-9-yl) methoxy]carbonyl}amino)-3-(6- fluoro-1,3-benzothiazol-2-yl) propanoic acid |
| (2S)-3-(1,3-benzoxazol-2- yl)-2-{[(9H-fluoren-9- ylmethoxy)carbonyl]amino} propanoic acid | 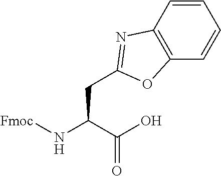 | (2S)-3-(1,3-benzoxazol-2-yl)-2- {[(9H-fluoren-9- ylmethoxy)carbonyl]amino} propanoic acid |
| (2S)-2-{[(9H-fluoren-9- ylmethoxy)carbonyl]amino}- 3-(6-methoxy-1,3- benzothiazol-2-yl)propanoic acid | 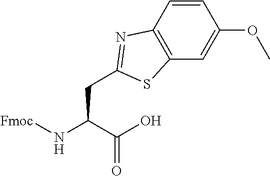 | (2S)-2-{[(9H-fluoren-9- ylmethoxy)carbonyl]amino}-3-(6- methoxy-1,3-benzothiazol-2- yl)propanoic acid |
| Fmoc-L-Ser(4′-OMe-Bip)- OH | 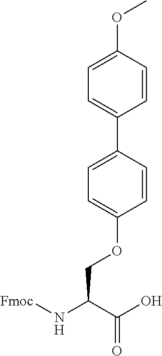 | (2S)-2-({[(9H-fluoren-9-yl) methoxy]carbonyl}amino)-3- ({4′-methoxy-[1,1′-bi-phenyl]-4- yl}oxy)propanoic acid |

TABLE 2
Free acids used for the synthesis of compounds of formula (I) (step 9, Scheme 1)
ACIDS

| Short Name | Structure | IUPAC Name |
|---|---|---|
| 2-methyloxazole-4- carboxylic acid | 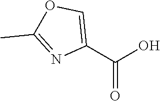 | 2-methyloxazole-4- carboxylic acid |
| 4-Fluorobenzoic acid | 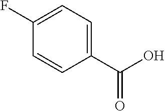 | 4-Fluorobenzoic acid |
| 7-Methoxycoumarin-3- carboxylic acid | 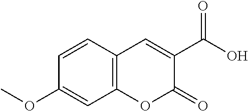 [SMILES: COc1ccc2cc(C(=O)O)c(=O)oc2c1] | 7-methoxy-2-oxo-2H- chromene-3-carboxylic acid |

TABLE 3
Primary or secondary amines used for the synthesis of compounds of formula (I) (step
10, Scheme 1).
AMINES

| Short Name | Structure | IUPAC Name |
|---|---|---|
| 1-[(dimethylamino)methyl] cyclobutan-1-amine | 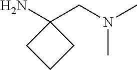 | 1-[(dimethylamino)methyl] cyclobutan-1-amine |
| (2-aminoethyl)dimethylamine | 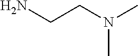 | (2-aminoethyl)dimethylamine |
| 2-(morpholin-4-yl)ethan-1- amine | 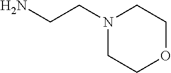 | 2-(morpholin-4-yl)ethan-1- amine |
| (2,2-difluoro-ethyl)-methyl- amine | 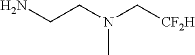 | (2,2-difluoro-ethyl)-methyl- amine |
| N-Boc-N-methylethylene diamine | 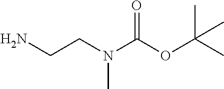 | tert-Butyl(2-aminoethyl) (methyl)carbamate |

### Example 2

### Synthesis of Amino Acids as Building Blocks for Compounds of Formula (I)

Some amino acids needed for the synthesis of compounds of formula (I) were not commercially available. In this case, they were synthesized. In Scheme 2 these syntheses are presented.

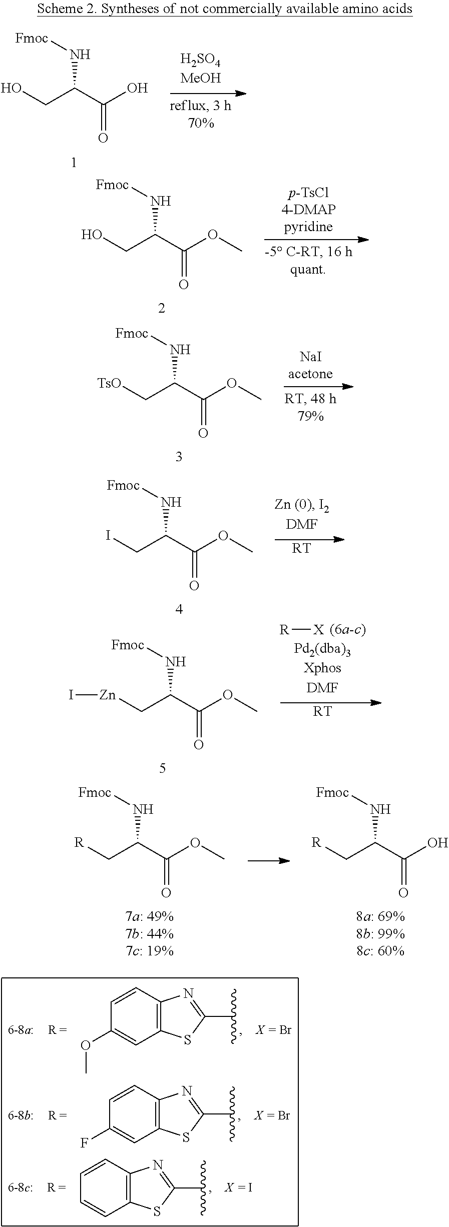

The synthesis of the amino acids 8a-c is depicted at Scheme 2. To begin with, FMOC-Ser-OH (1, 30.6 mmol, 1.0 equiv.) was suspended in MeOH (6 ml/mmol of 1) and drops of conc. H₂SO₄ were added. The reaction mixture was set under reflux for 3 h. Then it was cooled down to RT, the pH was adjusted to 8.0 using an aq. 20% w/v Na₂CO₃ sol. and extracted with EtOAc (3×150 ml). The organic layer was washed with brine, dried over MgSO₄, and evaporated to give 2 as a white solid.

In the next step, 2 (1.0 equiv.) and 4-DMAP (0.12 equiv.) were dissolved in dry pyridine (0.6 ml/mmol of 2) and cooled to −5° C. p-TsOH (3.3 equiv.) was added to the solution and the resultant yellow reaction mixture was stirred under nitrogen for 16 h. Then, the reaction was poured into ice-water and the aqueous mixture was extracted into ethyl acetate (3×100 mL). The combined organic layer was washed with water (2×100 ml), 10% w/v KHSO₄ solution (2×100 mL), sat. NaHCO₃ sol. (2×100 mL) and brine (2×100 ml). The organic layers were dried over Na₂SO₄, filtered, and concentrated under reduced pressure to give the tosylate 3. Following, a solution of sodium iodide (5.0 equiv.) in acetone (0.0 mL/mmol of NaI) was added dropwise to a solution of the tosylate 3 (1.0 equiv.) in acetone (1.0 mL/mmol of 3) under nitrogen. The resulting yellow solution was stirred for 48 h at RT. The reaction mixture was filtered, and the filtrate was concentrated under reduced pressure. The residue was dissolved in chloroform (100 mL), washed with water (2×50 mL), sat. Na₂S₂O₃ sol. (2×50 mL) and brine (2×30 mL). The organic layer was dried upon Na₂SO₄, filtered and concentrated under reduced pressure to give 4 a pale-yellow solid. Afterwards, Zn (0) (dust) (4.0 equiv.) was weighted into a flame-dried flask and I₂ (0.22 equiv.) dissolved in dry DMF (1.0 ml/mmol of Zn(0)) was added. Iodine 4 (1.0 equiv.) together with I₂ was added and the reaction mixture was stirred until full consumption (TLC indicated).

Most of the supernatant was taken out in a syringe and directly added to a pre-stirred solution of Pd₂(dba)₃ (0.05 mml), Xphos (0.10 mmol) and the respective halide 6a-c (1.3 mmol) in dry DMF (1.0 ml/mmol of Zn(0)). The reaction mixtures were stirred for 2 h at 40° C. Then the mixture was poured into sat. NaHCO₃ sol (10 ml) and extracted with EtOAc (2×10 ml). The combined organic layers were washed with sat. NaCl sol., dried over MgSO₄, concentrated, and dried under vacuum. The crude product was purified via flash column chromatography to furnish the esters 7a-c. Finally, to a suspension of 7a-c (1.0 equiv.) in 0.8 M CaCl₂ solution in ⁱPrOH/H₂O (20 ml/mmol of 7), NaOH (1.2 equiv.) was added and the mixture was stirred at RT overnight. The reaction was quenched by the addition of acetic acid and the solvent was removed under reduced pressure. The final amino acids 8a-c were obtained by the means of RP chromatography of the crude products.

### Example 3

### Synthesis of Compounds of Formula (I)

The synthesis of compounds of formula (I) has already described in EP 3′345′917 A1 including the synthesis of compounds 6025, 6027 and 6253. Analogously, except if mentioned otherwise, the compounds of formula (I) further described thereafter have been synthesized according to the general procedure depicted in Scheme 1. Each of the synthesized compounds are identified by its compound number (4- or 5-digit number), its structural formula and its IUPAC name as generated by the MarvinSketch software. Furthermore, the synthesis of 6328, 6781, 7058, 7059, 7824, 7844, 7848, 7850, 8341, 8342, 8343, 21164, 21189, 21287, 21314, 21315, 21359, 21362, 21365, 21368, 21369 and 21372 is described by way of the building blocks, acids and amines used as disclosed in Tables 1-3. Moreover, the experimentally measured molecular mass is provided. Finally, for each specifically identified compound of formula (I) the characterization of the substituents R₁, R₂, and R₃ in accordance with formula (I) is given, wherein the residue R within said definition of said substituents R₁, R₂, and R₃ corresponds to and indicates the respective attachment within formula (I).

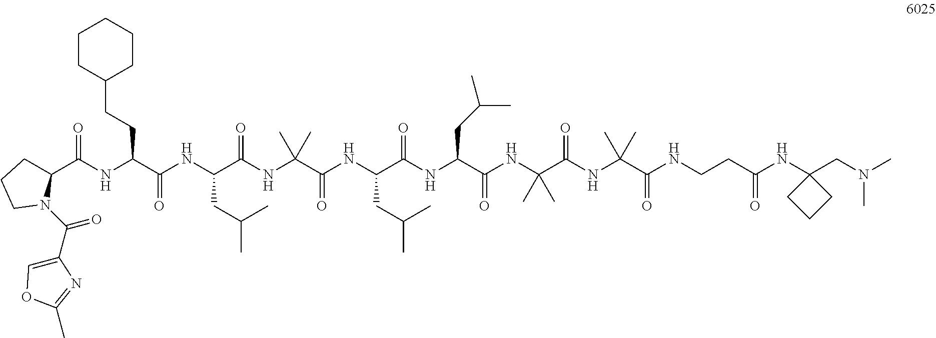 [SMILES: Cc1nc(C(=O)N2CCC[C@H]2C(=O)N[C@@H](CCC2CCCCC2)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC2(CN(C)C)CCC2)co1]

### (2S)-2-[(2S)-4-cyclohexyl-2-{[(2S)-1-(2-methyl-1,3-oxazole-4-carbonyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide

| Cpd | R1 | R2 | R3 |
|---|---|---|---|
| 6025 | 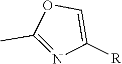 | 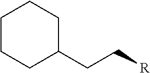 | 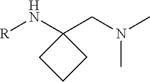 |

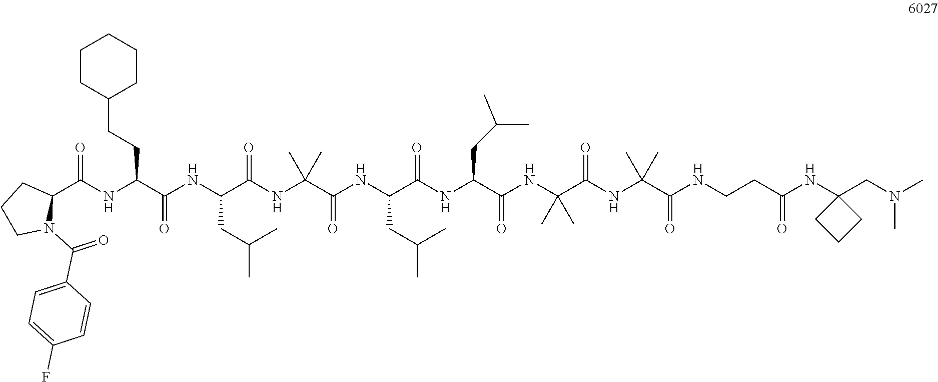 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC1(CN(C)C)CCC1]

### (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide

| Cpd | R1 | R2 | R3 |
|---|---|---|---|
| 6027 | 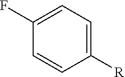 | 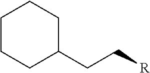 | 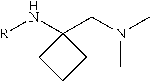 |

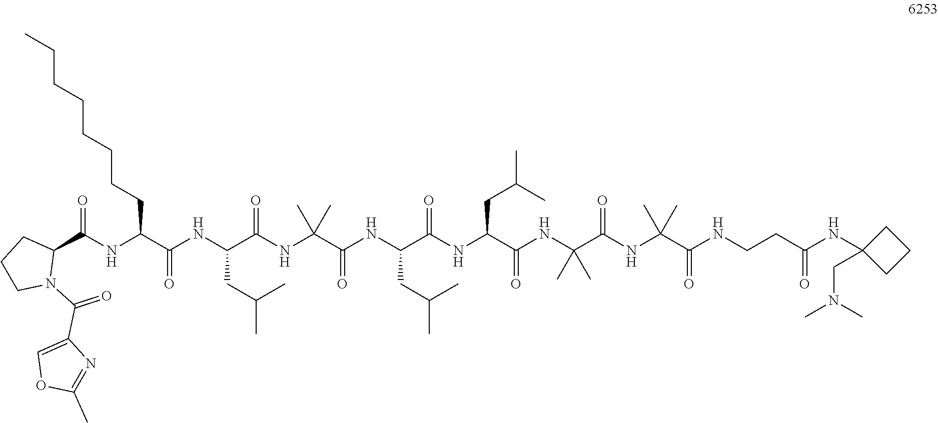 [SMILES: CCCCCCCC[C@H](NC(=O)[C@@H]1CCCN1C(=O)c1coc(C)n1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC1(CN(C)C)CCC1]

### (2S)—N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}decanamide

| Cpd | R1 | R2 | R3 |
|---|---|---|---|
| 6253 | 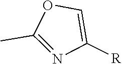 | 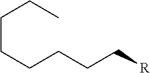 | 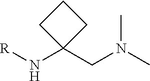 |

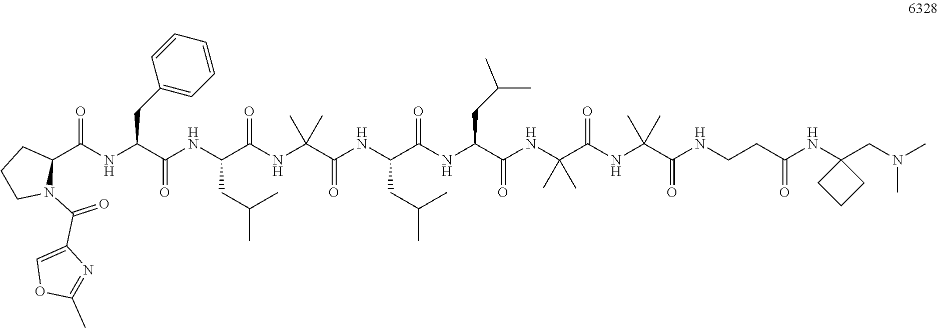 [SMILES: Cc1nc(C(=O)N2CCC[C@H]2C(=O)N[C@@H](Cc2ccccc2)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC2(CN(C)C)CCC2)co1]

### (2S)—N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methyl-2-[(2S)-2-{[(2S)-1-(2-methyl-1,3-oxazole-4-carbonyl)pyrrolidin-2-yl]formamido}-3-phenylpropanamido]pentanamide

| Cpd | 1 | 2 | 3 | 4 | 5 |
|---|---|---|---|---|---|
| 6328 | Fmoc- | Fmoc- | Fmoc- | Fmoc-Leu-OH | Fmoc-Aib-OH |
| | Aib-OH | Aib-OH | Leu-OH | | |
| | 6 | 7 | 8 | 9 | 10 |
| | Fmoc- | Fmoc- | Fmoc- | 2- | 1-[(dimethylamino) |
| | Leu-OH | Phe-OH | Pro-OH | methyloxazole- | methyl]cyclobutan- |
| | | | | 4-carboxylic | 1-amine |
| | | | | acid | |

| | Molecular | Experimentally |
|---|---|---|
| Cpd | mass | measured mass |
| 6328 | 1147.72 | 1147.76 |
| [M + H] + | | |

| Cpd | R1 | R2 | R3 |
|---|---|---|---|
| 6328 | 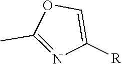 | 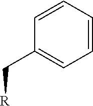 | 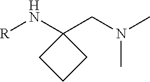 |

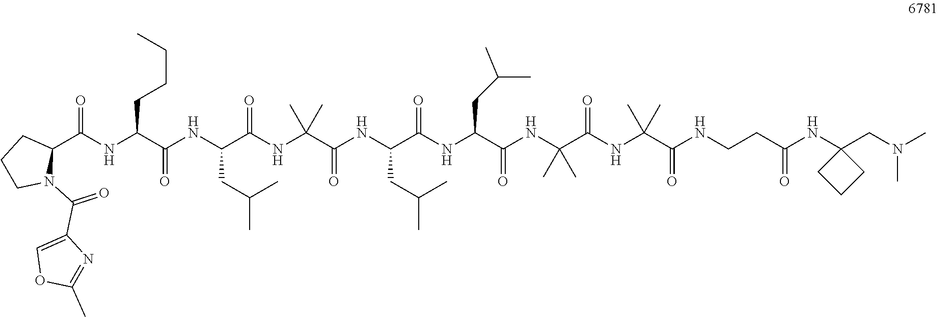 [SMILES: CCCC[C@H](NC(=O)[C@@H]1CCCN1C(=O)c1coc(C)n1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC1(CN(C)C)CCC1]

### (2S)—N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}hexanamide

| Cpd | 1 | 2 | 3 | 4 | 5 |
|---|---|---|---|---|---|
| 6781 | Fmoc-Aib-OH | Fmoc-Aib-OH | Fmoc-Leu-OH | Fmoc-Leu-OH | Fmoc-Aib-OH |
| | 6 | 7 | 8 | 9 | 10 |
| | Fmoc-Leu-OH | Fmoc-Nle-OH | Fmoc-Pro-OH | 2-methyloxazole- | 1-[(dimethylamino) |
| | | | | 4-carboxylic acid | methyl]cyclobutan- |
| | | | | | 1-amine |

| Cpd | Molecular mass | Experimentally measured mass |
|---|---|---|
| 6781 [M + H]+ | 1113.74 | 1113.60 |

| Cpd | R1 | R2 | R3 |
|---|---|---|---|
| 6781 | 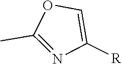 | 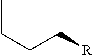 | 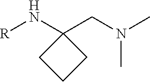 |

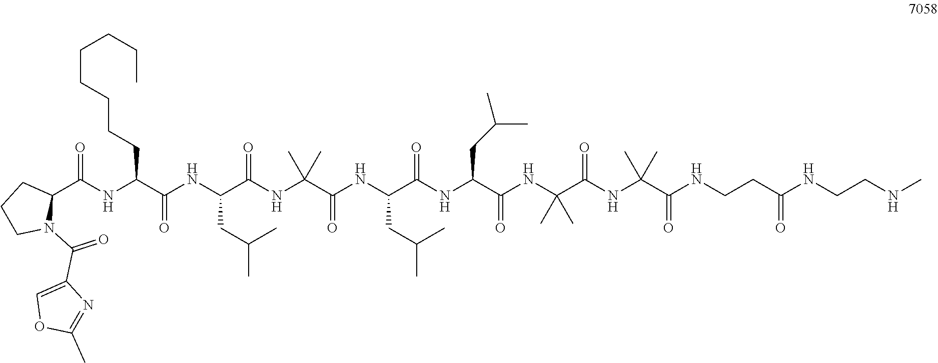 [SMILES: CCCCCCCC[C@H](NC(=O)[C@@H]1CCCN1C(=O)c1coc(C)n1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCNC]

### (2S)-2-{[(2S)-1-(2-methyl-1,3-oxazole-4-carbonyl)pyrrolidin-2-yl]formamido}-N-[(1S)-3-methyl-1-[(1-methyl-1-{[(1S)-3-methyl-1-{[(1S)-3-methyl-1-{[1-methyl-1-({1-methyl-1-[(2-{[2-(methylamino)ethyl]carbamoyl}ethyl)carbamoyl]ethyl}carbamoyl) ethyl]carbamoyl}butyl]carbamoyl}butyl]carbamoyl}ethyl)carbamoyl]butyl]decanamide

| Cpd | 1 | 2 | 3 | 4 | 5 |
|---|---|---|---|---|---|
| 7058 | Fmoc-Aib-OH | Fmoc-Aib-OH | Fmoc-Leu-OH | Fmoc-Leu-OH | Fmoc-Aib-OH |
| | 6 | 7 | 8 | 9 | 10 |
| | Fmoc-Leu-OH | Fmoc-(S)-2- | Fmoc-Pro-OH | 2-methyloxazole- | tert-Butyl (2- |
| | | amino-decanoic | | 4-carboxylic acid | aminoethyl) |
| | | acid | | | (methyl) |
| | | | | | carbamate |

| Cpd | Molecular mass | Experimentally measured mass |
|---|---|---|
| 7058 [M + H] + | 1115.76 | 1115.70 |

| Cpd | R1 | R2 | R3 |
|---|---|---|---|
| 7058 | 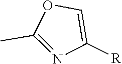 | 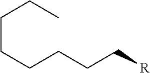 | 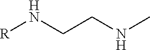 |

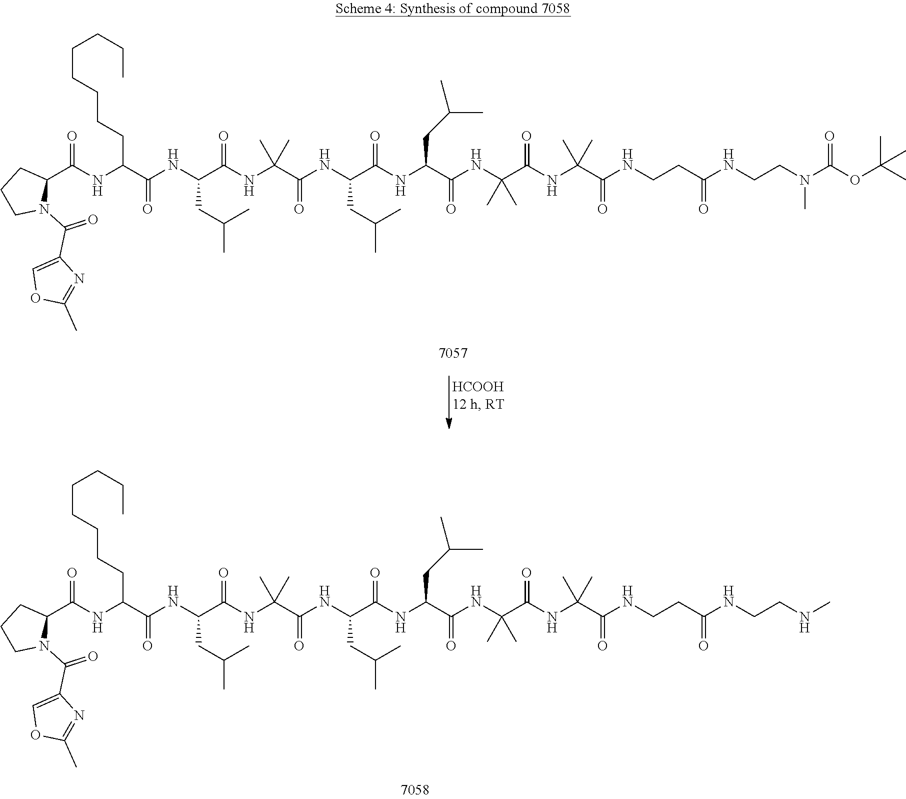 [SMILES: CCCCCCCCC(NC(=O)[C@@H]1CCCN1C(=O)c1coc(C)n1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCN(C)C(=O)OC(C)(C)C ; CCCCCCCCC(NC(=O)[C@@H]1CCCN1C(=O)c1coc(C)n1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCNC]

Compound 7058 was produced as depicted in Scheme 4. More analytically: Formic acid (3.7 mL) was added at compound 7057 (0.1 mmol) and the reaction mixture was left to stir for 12 h at RT. Upon completion, formic acid was removed under vacuo and 7058 was obtained without further purification.

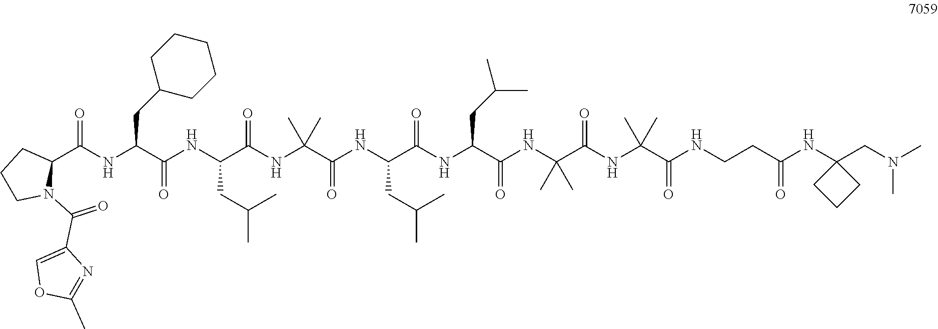 [SMILES: Cc1nc(C(=O)N2CCC[C@H]2C(=O)N[C@@H](CC2CCCCC2)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC2(CN(C)C)CCC2)co1]

### (2S)-2-[(2S)-3-cyclohexyl-2-{[(2S)-1-(2-methyl-1,3-oxazole-4-carbonyl)pyrrolidin-2-yl]formamido}propanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide

| Cpd | 1 | 2 | 3 | 4 | 5 |
|---|---|---|---|---|---|
| 7059 | Fmoc-Aib-OH | Fmoc-Aib-OH | Fmoc-Leu-OH | Fmoc-Leu-OH | Fmoc-Aib-OH |
| | 6 | 7 | 8 | 9 | 10 |
| | Fmoc-Leu-OH | Fmoc-Cha-OH | Fmoc-Pro-OH | 2-methyloxazole-4- | 1-[(dimethylamino) |
| | | | | carboxylic acid | methyl]cyclobutan- |
| | | | | | 1-amine |

| Cpd | Molecular mass | Experimentally measured mass |
|---|---|---|
| 7059 [M + H] + | 1153.77 | 1153.50 |

| Cpd | R1 | R2 | R3 |
|---|---|---|---|
| 7059 | 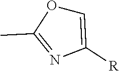 | 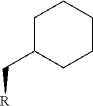 | 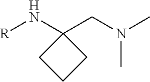 |

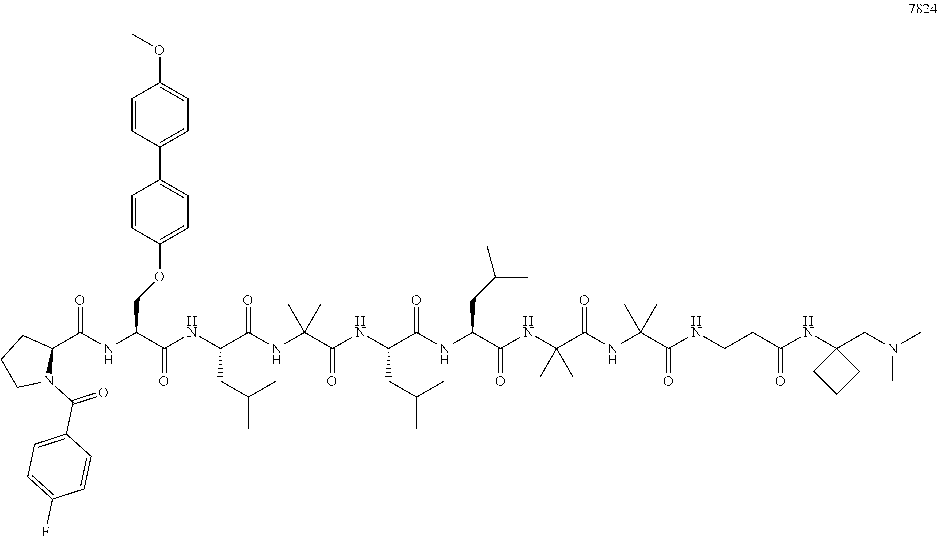 [SMILES: COc1ccc(-c2ccc(OC[C@H](NC(=O)[C@@H]3CCCN3C(=O)c3ccc(F)cc3)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC3(CN(C)C)CCC3)cc2)cc1]

### (2S)—N-{1-[(1-{[2-({1-[(dimethylamino)methyl]cyclobutyl}carbamoyl)ethyl]carbamoyl}-1-methylethyl)carbamoyl]-1-methylethyl}-2-[(2S)-2-{2-[(2S)-2-[(2S)-2-{[(2S)-1-(4-fluorobenzoyl)pyrrolidin-2-yl]formamido}-3-({4′-methoxy-[1,1′-biphenyl]-4-yl}oxy)propanamido]-4-methylpentanamido]-2-methylpropanamido}-4-methylpentanamido]-4-methylpentanamide

| Cpd | 1 | 2 | 3 | 4 | 5 |
|---|---|---|---|---|---|
| 7824 | Fmoc-Aib-OH | Fmoc-Aib-OH | Fmoc-Leu-OH | Fmoc-Leu-OH | Fmoc-Aib-OH |
| | 6 | 7 | 8 | 9 | 10 |
| | Fmoc-Leu-OH | Fmoc-L-Ser(4′- | Fmoc-Pro-OH | 4- | 1-[(dimethylamino) |
| | | OMe-Bip)-OH | | Fluorobenzoic | methyl]cyclobutan- |
| | | | | acid | 1-amine |

| Cpd | Molecular mass | Experimentally measured mass |
|---|---|---|
| 7824 [M + H]+ | 1282.76 | 1282.51 |

| Cpd | R1 | R2 | R3 |
|---|---|---|---|
| 7824 | 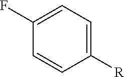 | 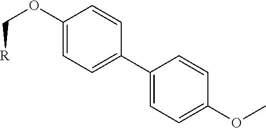 | 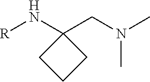 |

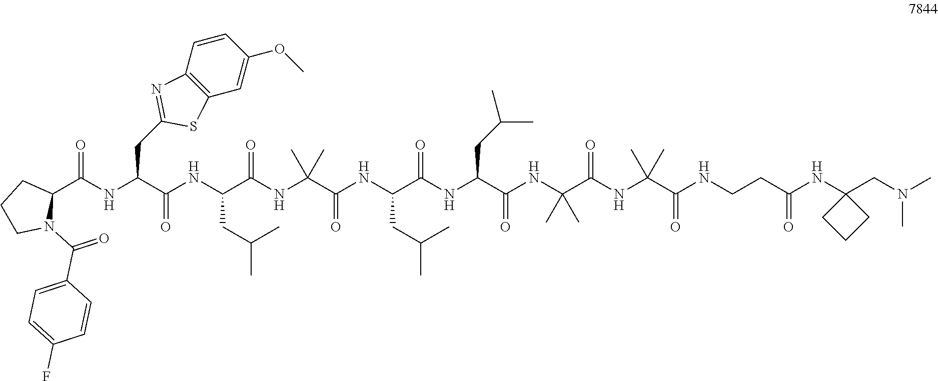 [SMILES: COc1ccc2nc(C[C@H](NC(=O)[C@@H]3CCCN3C(=O)c3ccc(F)cc3)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC3(CN(C)C)CCC3)sc2c1]

### (2S)—N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-2-{[(2S)-1-(4-fluorobenzoyl)pyrrolidin-2-yl]formamido}-3-(6-methoxy-1,3-benzothiazol-2-yl)propanamido]-4-methylpentanamide

| Cpd | 1 | 2 | 3 | 4 | 5 |
|---|---|---|---|---|---|
| 7844 | Fmoc-Aib-OH | Fmoc-Aib-OH | Fmoc-Leu-OH | Fmoc-Leu-OH | Fmoc-Aib-OH |
| | 6 | 7 | 8 | 9 | 10 |
| | Fmoc-Leu-OH | (2S)-2-{[(9H-fluoren-9- | Fmoc-Pro-OH | 4- | 1-[(dimethylamino) |
| | | ylmethoxy)carbonyl]amino}- | | Fluorobenzoic | methyl]cyclobutan- |
| | | 3-(6-methoxy-1,3- | | acid | 1-amine |
| | | benzothiazol-2-yl)propanoic | | | |
| | | acid | | | |

| Cpd | Molecular mass | Experimentally measured mass |
|---|---|---|
| 7844 [M + H]+ | 1247.58 | 1248.34 |

| | R1 | R2 | R3 |
|---|---|---|---|
| 7844 | 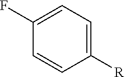 | 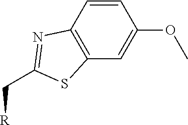 | 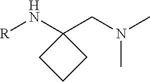 |

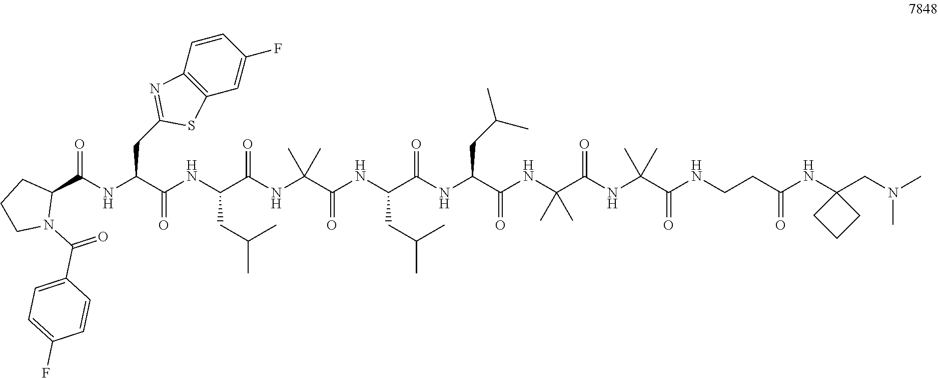 [SMILES: CC(C)C[C@H](NC(=O)[C@H](Cc1nc2ccc(F)cc2s1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC1(CN(C)C)CCC1]

### (2S)—N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-3-(6-fluoro-1,3-benzothiazol-2-yl)-2-{[(2S)-1-(4-fluorobenzoyl)pyrrolidin-2-yl]formamido}propanamido]-4-methylpentanamide

| Cpd | 1 | 2 | 3 | 4 | 5 |
|---|---|---|---|---|---|
| 7848 | Fmoc-Aib-OH | Fmoc-Aib-OH | Fmoc-Leu-OH | Fmoc-Leu-OH | Fmoc-Aib-OH |
| | 6 | 7 | 8 | 9 | 10 |
| | Fmoc-Leu-OH | (2S)-2-({[(9H-fluoren-9- | Fmoc-Pro-OH | 4- | 1-[(dimethylamino) |
| | | yl)methoxy]carbonyl}amino)- | | Fluorobenzoic | methyl]cyclobutan- |
| | | 3-(6-fluoro-1,3-benzothia- | | acid | 1-amine |
| | | zol-2-yl)propanoic acid | | | |

| Cpd | Molecular mass | Experimentally measured mass |
|---|---|---|
| 7848 [M + H]+ | 1235.68 | 1236.38 |

| | R1 | R2 | R3 |
|---|---|---|---|
| 7848 | 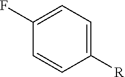 | 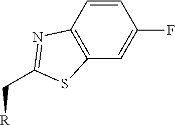 | 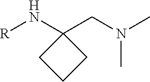 |

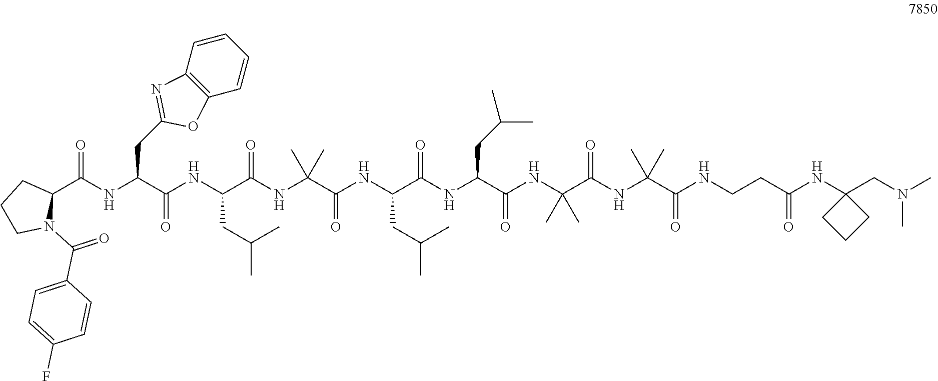 [SMILES: CC(C)C[C@H](NC(=O)[C@H](Cc1nc2ccccc2o1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC1(CN(C)C)CCC1]

### (2S)-2-[(2S)-3-(1,3-benzoxazol-2-yl)-2-{[(2S)-1-(4-fluorobenzoyl)pyrrolidin-2-yl]formamido}propanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide

| Cpd | 1 | 2 | 3 | 4 | 5 |
|---|---|---|---|---|---|
| 7850 | Fmoc-Aib-OH | Fmoc-Aib-OH | Fmoc-Leu-OH | Fmoc-Leu-OH | Fmoc-Aib-OH |
| | 6 | 7 | 8 | 9 | 10 |
| | Fmoc-Leu-OH | (2S)-3-(1,3-benzoxazol-2- | Fmoc-Pro-OH | 4- | 1-[(dimethylamino) |
| | | yl)-2-{[(9H-fluoren-9- | | Fluorobenzoic | methyl]cyclobutan- |
| | | ylmethoxy)carbonyl]amino} | | acid | 1-amine |
| | | propanoic acid | | | |

| Cpd | Molecular mass | Experimentally measured mass |
|---|---|---|
| 7850 [M + H]+ | 1235.68 | 1236.38 |

| Cpd | R₁ | R₂ | R₃ |
|---|---|---|---|
| 7850 | 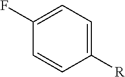 | 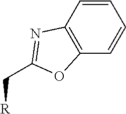 | 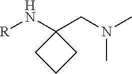 |

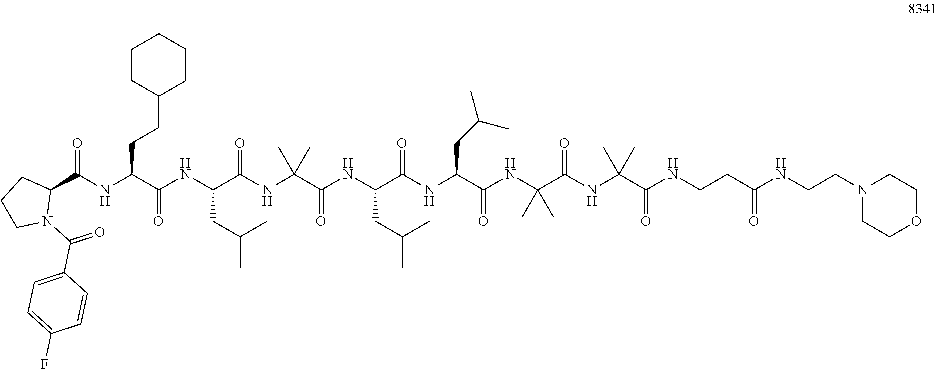 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCN1CCOCC1]

### (2S)-2-[(2S)-4-cyclohexyl-2-{[(2S)-1-(4-fluorobenzoyl)pyrrolidin-2-yl]formamido}butanamido]-4-methyl-N-(1-methyl-1-{[(1S)-3-methyl-1-{[(1S)-3-methyl-1-{[1-methyl-1-({1-methyl-1-[(2-{[2-(morpholin-4-yl)ethyl]carbamoyl}ethyl)carbamoyl]ethyl}carbamoyl)ethyl]carbamoyl}butyl]carbamoyl}butyl]carbamoyl}ethyl)pentanamide

| Cpd | 1 | 2 | 3 | 4 | 5 |
|---|---|---|---|---|---|
| 8341 | Fmoc-Aib-OH | Fmoc-Aib-OH | Fmoc-Leu-OH | Fmoc-Leu-OH | Fmoc-Aib-OH |
| | 6 | 7 | 8 | 9 | 10 |
| | Fmoc-Leu-OH | Fmoc-L- | Fmoc-Pro-OH | 4- | 2-(morpholin- |
| | | homocyclohexyl- | | Fluorobenzoic | 4-yl)ethan-1- |
| | | alanine | | acid | amine |

| Cpd | Molecular mass | Experimentally measured mass |
|---|---|---|
| 8341 [M + H]+ | 1182.77 | 1183.04 |

| Cpd | R1 | R2 | R3 |
|---|---|---|---|
| 8341 | 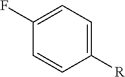 | 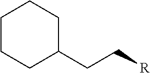 | 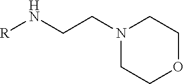 |

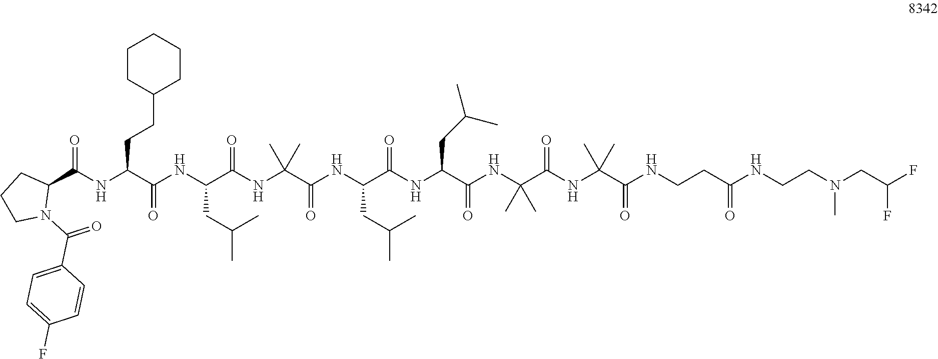 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCN(C)CC(F)F]

### (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({2-[(2,2-difluoroethyl)(methyl)amino]ethyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide

| Cpd | 1 | 2 | 3 | 4 | 5 |
|---|---|---|---|---|---|
| 8342 | Fmoc-Aib-OH | Fmoc-Aib-OH | Fmoc-Leu-OH | Fmoc-Leu-OH | Fmoc-Aib-OH |
| | 6 | 7 | 8 | 9 | 10 |
| | Fmoc-Leu-OH | Fmoc-L- | Fmoc-Pro-OH | 4-Fluorobenzoic | (2,2-difluoro- |
| | | homocyclohexyl- | | acid | ethyl)-methyl- |
| | | alanine | | | amine |

| Cpd | Molecular mass | Experimentally measured mass |
|---|---|---|
| 8342 [M + H]+ | 1190.75 | 1191.09 |

| Cpd | R1 | R2 | R3 |
|---|---|---|---|
| 8342 | 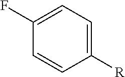 | 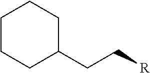 | 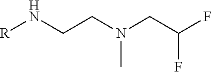 |

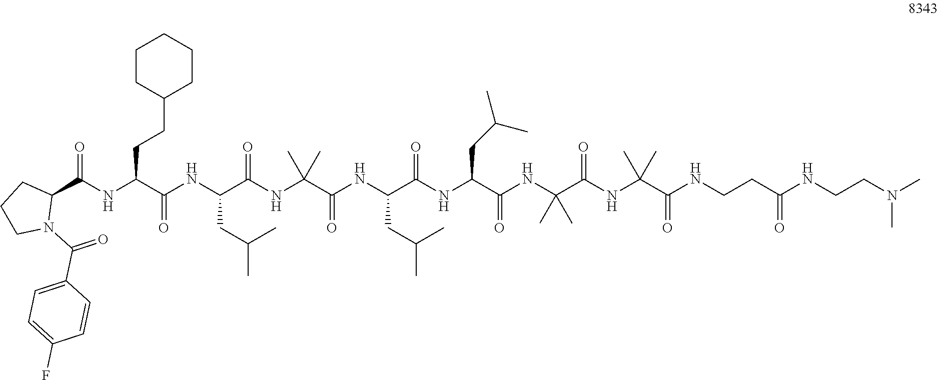 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCN(C)C]

### (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-{[1-({1-[(2-{[2-(dimethylamino)ethyl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)-4-methylpentanamide

| Cpd | 1 | 2 | 3 | 4 | 5 |
|---|---|---|---|---|---|
| 8343 | Fmoc-Aib-OH | Fmoc-Aib-OH | Fmoc-Leu-OH | Fmoc-Leu-OH | Fmoc-Aib-OH |
| | 6 | 7 | 8 | 9 | 10 |
| | Fmoc-Leu-OH | Fmoc-L- | Fmoc-Pro-OH | 4- | (2-aminoethyl) |
| | | homocyclohexyl- | | Fluorobenzoic | dimethylamine |
| | | alanine | | acid | |

| Cpd | Molecular mass | Experimentally measured mass |
|---|---|---|
| 8343 [M + H]+ | 1140.75 | 1141.04 |

| Cpd | R₁ | R₂ | R₃ |
|---|---|---|---|
| 8343 | 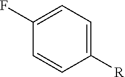 | 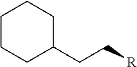 | 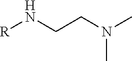 |

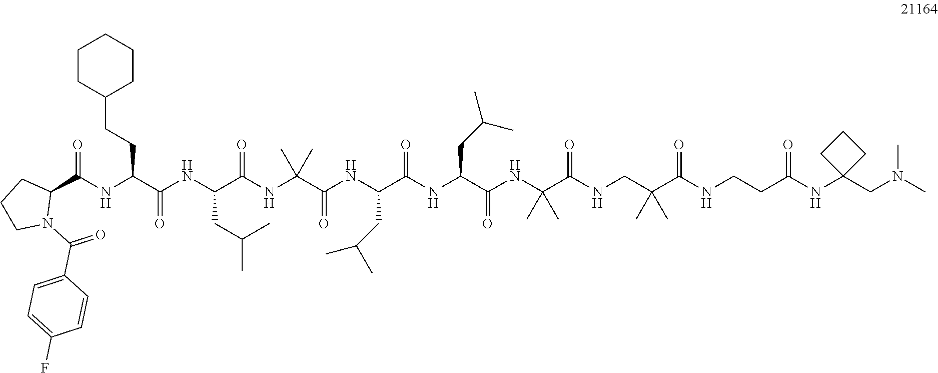 [SMILES: CC(C)C[C@H](NC(=O)[C@H](CC(C)C)NC(=O)C(C)(C)NC(=O)[C@H](CC(C)C)NC(=O)[C@H](CCC1CCCCC1)NC(=O)[C@@H]1CCCN1C(=O)c1ccc(F)cc1)C(=O)NC(C)(C)C(=O)NCC(C)(C)C(=O)NCCC(=O)NC1(CN(C)C)CCC1]

### (2S)-2-[(2S)-4-cyclohexyl-2-{[(2S)-1-(4-fluorobenzoyl)pyrrolidin-2-yl]formamido}butan-amido]-N-(1-{[(1S)-1-{[(1S)-1-({1-[(2-{[2-({1-[(dimethylamino)methyl]cyclobutyl}carba-moyl)ethyl]carbamoyl}-2,2-dimethylethyl)carbamoyl]-1-methylethyl}carbamoyl)-3-methylbutyl]carbamoyl}1-3-methylbutyl]carbamoyl}-1-methylethyl)-4-methylpentanamide

| Cpd | 1 | 2 | 3 | 4 | 5 |
|---|---|---|---|---|---|
| 21164 | Fmoc-3- | Fmoc-Aib-OH | Fmoc-Leu-OH | Fmoc-Leu-OH | Fmoc-Aib-OH |
| | amino-2,2- | | | | |
| | dimethyl- | | | | |
| | propionic acid | | | | |
| | 6 | 7 | 8 | 9 | 10 |
| | Fmoc-Leu-OH | Fmoc-L- | Fmoc-Pro-OH | 4- | 1-[(dimethyl- |
| | | homocyclohexyl- | | Fluorobenzoic | amino)methyl] |
| | | alanine | | acid | cyclobutan-1- |
| | | | | | amine |

| Cpd | Molecular mass | Experimentally measured mass |
|---|---|---|
| 21164 [M + H]+ | 1195.60 | 1195.13 |

| Cpd | R₁ | R₂ | R₃ |
|---|---|---|---|
| 21164 | 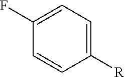 | 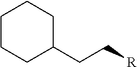 | 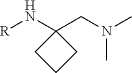 |

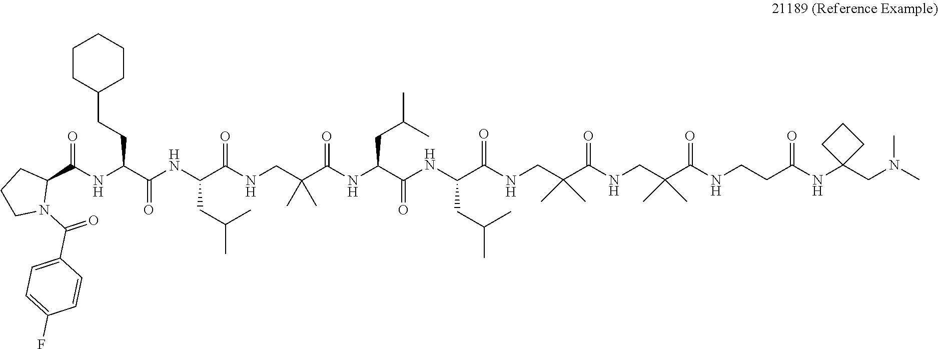 [SMILES: CC(C)C[C@H](NC(=O)[C@H](CCC1CCCCC1)NC(=O)[C@@H]1CCCN1C(=O)c1ccc(F)cc1)C(=O)NCC(C)(C)C(=O)N[C@@H](CC(C)C)C(=O)N[C@@H](CC(C)C)C(=O)NCC(C)(C)C(=O)NCC(C)(C)C(=O)NCCC(=O)NC1(CN(C)C)CCC1]

### (2S)-2-[(2S)-4-cyclohexyl-2-{[(2S)-1-(4-fluorobenzoyl)pyrrolidin-2-yl]formamido}butan-amido]-N-(2-{[(1S)-1-{[(1S)-1-({2-[(2-{[2-({1-[(dimethylamino)methyl]cyclobutyl}carbamoyl)ethyl]carbamoyl}-2,2-dimethylethyl)carbamoyl]-2,2-dimethylethyl}carbamoyl)-3-methylbutyl]carbamoyl}-3-methylbutyl]carbamoyl}-2,2-dimethylethyl)-4-methylpentanamide

| Cpd | 1 | 2 | 3 | 4 | 5 |
|---|---|---|---|---|---|
| 21189 | Fmoc-3- | Fmoc-3-amino-2,2- | Fmoc-Leu-OH | Fmoc-Leu-OH | Fmoc-3-amino- |
| | amino-2,2- | dimethyl-propionic | | | 2,2-dimethyl- |
| | dimethyl- | acid | | | propionic acid |
| | propionic acid | | | | |
| | 6 | 7 | 8 | 9 | 10 |
| | Fmoc-Leu-OH | Fmoc-L- | Fmoc-Pro-OH | 4- | 1-[(dimethyl- |
| | | homocyclohexyl- | | Fluorobenzoic | amino)methyl] |
| | | alanine | | acid | cyclobutan-1- |
| | | | | | amine |

| Cpd | Molecular mass | Experimentally measured mass |
|---|---|---|
| 21189 [M + H]+ | 1123.65 | 1123.02 |

| Cpd | R₁ | R₂ | R₃ |
|---|---|---|---|
| 21189 | 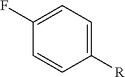 | 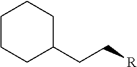 | 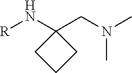 |

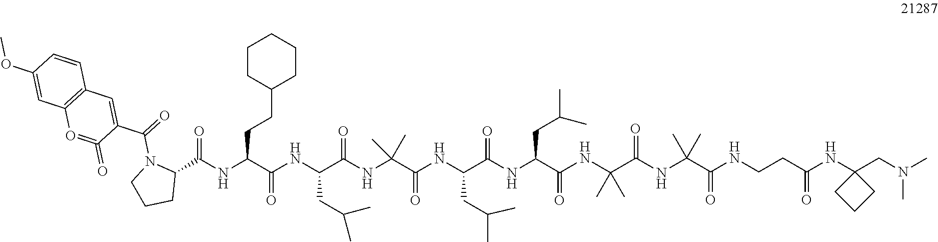

### (2S)-2-[(2S)-4-cyclohexyl-2-{[(2S)-1-(7-methoxy-2-oxo-2H-chromene-3-carbonyl) pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide

| Cpd | 1 | 2 | 3 | 4 | 5 |
|---|---|---|---|---|---|
| 21287 | Fmoc-Aib-OH | Fmoc-Aib-OH | Fmoc-Leu-OH | Fmoc-Leu-OH | Fmoc-Aib-OH |
| | 6 | 7 | 8 | 9 | 10 |
| | Fmoc-Leu-OH | Fmoc-L- | Fmoc-Pro-OH | 7-Methoxy- | 1-[(dimethyl- |
| | | homocyclohexyl- | | coumarin-3- | amino)methyl] |
| | | alanine | | carboxylic acid | cyclobutan-1- |
| | | | | | amine |

| Cpd | Molecular mass | Experimentally measured mass |
|---|---|---|
| 21287 [M + H]+ | 1261.64 | 1261.22 |

| Cpd | R₁ | R₂ | R₃ |
|---|---|---|---|
| 21287 | 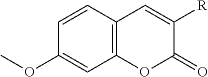 | 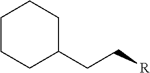 | 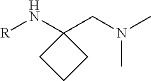 |

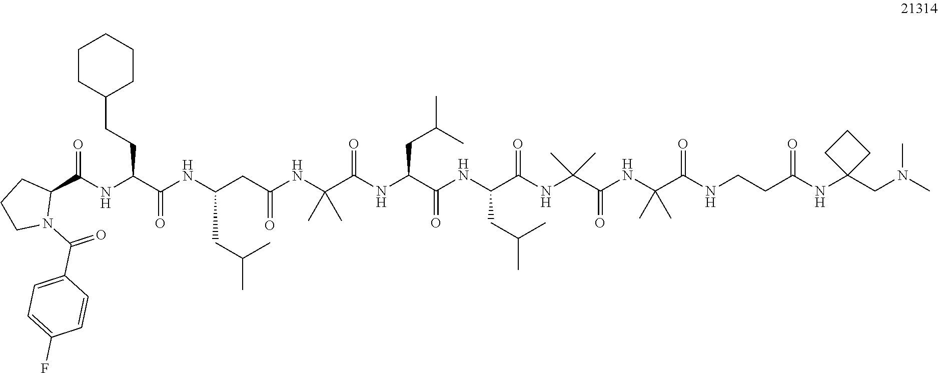 [SMILES: CC(C)C[C@@H](CC(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC1(CN(C)C)CCC1)NC(=O)[C@H](CCC1CCCCC1)NC(=O)[C@@H]1CCCN1C(=O)c1ccc(F)cc1]

### (3S)-3-[(2S)-4-cyclohexyl-2-{[(2S)-1-(4-fluorobenzoyl)pyrrolidin-2-yl]formamido}butan-amido]-N-(1-{[(1S)-1-{[(1S)-1-({1-[(1-{[2-({1-[(dimethylamino)methyl]cyclobutyl}carba-moyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-5-methylhexanamide

| Cpd | 1 | 2 | 3 | 4 | 5 |
|---|---|---|---|---|---|
| 21314 | Fmoc-Aib-OH | Fmoc-Aib-OH | Fmoc-Leu-OH | Fmoc-Leu-OH | Fmoc-Aib-OH |
| | 6 | 7 | 8 | 9 | 10 |
| | Fmoc-β- | Fmoc-L- | Fmoc-Pro-OH | 4- | 1-[(dimethyl- |
| | Homoleu-OH | homocyclohexyl- | | Fluorobenzoic | amino)methyl] |
| | | alanine | | acid | cyclobutan-1- |
| | | | | | amine |

| Cpd | Molecular mass | Experimentally measured mass |
|---|---|---|
| 21314 [M + H]+ | 1195.60 | 1195.13 |

| Cpd | R₁ | R₂ | R₃ |
|---|---|---|---|
| 21314 | 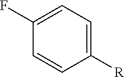 | 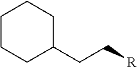 | 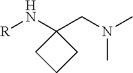 |

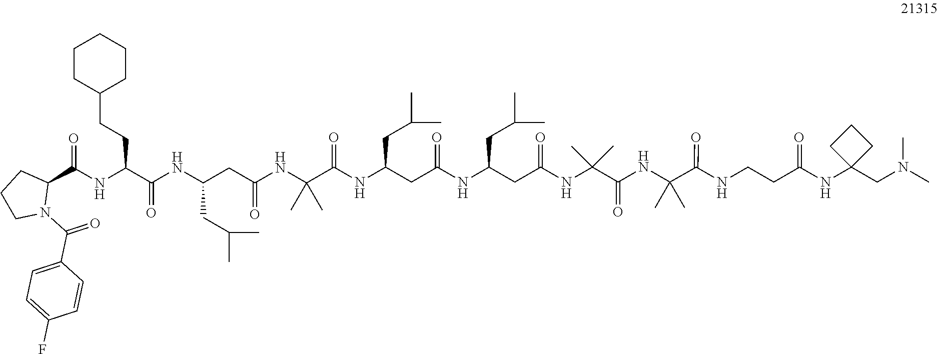 [SMILES: CC(C)C[C@@H](CC(=O)NC(C)(C)C(=O)NC(C)(C)C(=O)NCCC(=O)NC1(CN(C)C)CCC1)NC(=O)C[C@H](CC(C)C)NC(=O)C(C)(C)NC(=O)C[C@H](CC(C)C)NC(=O)[C@H](CCC1CCCCC1)NC(=O)[C@@H]1CCCN1C(=O)c1ccc(F)cc1]

### (3S)-3-[(2S)-4-cyclohexyl-2-{[(2S)-1-(4-fluorobenzoyl)pyrrolidin-2-yl]formamido}butanamido]-N-(1-{[(2S)-1-{[(2S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-4-methylpentan-2-yl]carbamoyl}-4-methylpentan-2-yl]carbamoyl}-1-methylethyl)-5-methylhexanamide

| Cpd | 1 | 2 | 3 | 4 | 5 |
|---|---|---|---|---|---|
| 21315 | Fmoc-Aib-OH | Fmoc-Aib-OH | Fmoc-β- | Fmoc-β- | Fmoc-Aib-OH |
| | | | Homoleu-OH | Homoleu-OH | |
| | 6 | 7 | 8 | 9 | 10 |
| | Fmoc-β- | Fmoc-L- | Fmoc-Pro-OH | 4- | 1-[(dimethyl- |
| | Homoleu-OH | homocyclohexyl- | | Fluorobenzoic | amino)methyl] |
| | | alanine | | acid | cyclobutan-1- |
| | | | | | amine |

| Cpd | Molecular mass | Experimentally measured mass |
|---|---|---|
| 21315 [M + H]+ | 1223.65 | 1223.49 |

| Cpd | R₁ | R₂ | R₃ |
|---|---|---|---|
| 21315 | 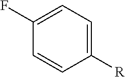 | 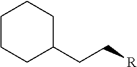 | 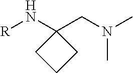 |

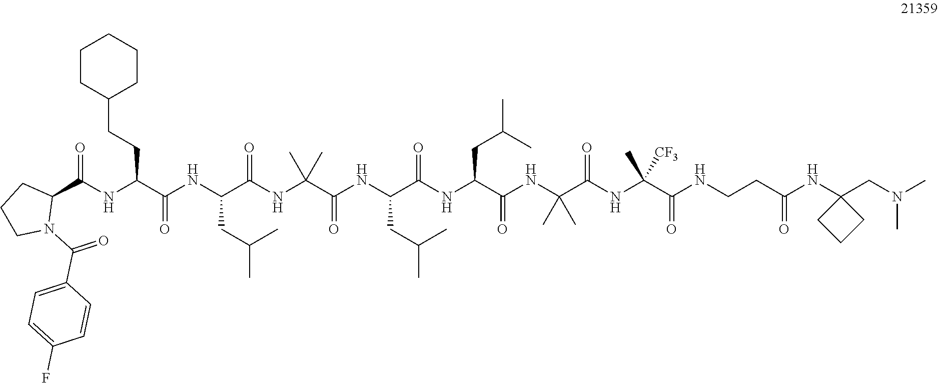 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)N[C@@](C)(C(=O)NCCC(=O)NC1(CN(C)C)CCC1)C(F)(F)F]

### (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-[(1-{[(1S)-1-{[2-({1-[(dimethylamino)methyl]cyclobutyl}carbamoyl)ethyl]carbamoyl}-2,2,2-trifluoro-1-methylethyl]carbamoyl}-1-methylethyl)carbamoyl]-3-methylbutyl]carbamoyl}-3-methylbutyl]carbamoyl}-1-methylethyl)-4-methylpentanamide

| Cpd | 1 | 2 | 3 | 4 | 5 |
|---|---|---|---|---|---|
| 21359 | Fmoc- | Fmoc-alpha- | Fmoc- | Fmoc- | Fmoc- |
| | Aib-OH | Tfm-L-Ala-OH | Leu-OH | Leu-OH | Aib-OH |
| | 6 | 7 | 8 | 9 | 10 |
| | Fmoc- | Fmoc-L- | Fmoc- | 4-Fluorobenzoic | 1-[(dimethyl- |
| | Leu-OH | homocyclohexyl | Pro-OH | acid | amino)methyl] |
| | | alanine | | | cyclobutan- |
| | | | | | 1-amine |

| | Molecular | Experimentally |
|---|---|---|
| Cpd | mass | measured mass |
| 21359 | 1166.53 | 1166.71 |
| [M + H—CF₃]+ | | |

| Cpd | R1 | R2 | R3 |
|---|---|---|---|
| 21359 | 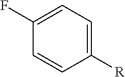 | 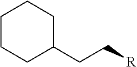 | 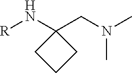 |

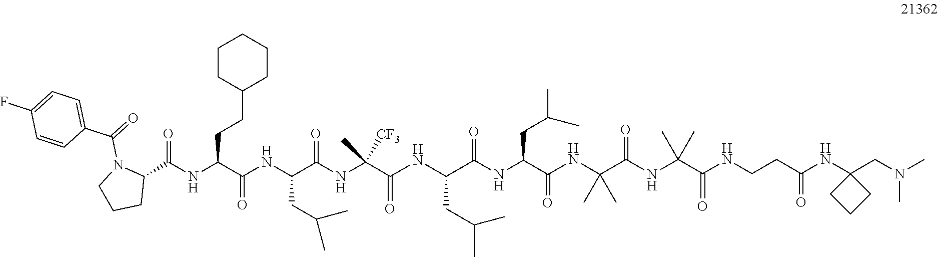 [SMILES: CC(C)C[C@H](NC(=O)[C@H](CC(C)C)NC(=O)[C@](C)(NC(=O)[C@H](CC(C)C)NC(=O)[C@H](CCC1CCCCC1)NC(=O)[C@@H]1CCCN1C(=O)c1ccc(F)cc1)C(F)(F)F)C(=O)NC(C)(C)C(=O)NC(C)(C)C(=O)NCCC(=O)NC1(CN(C)C)CCC1]

### (2S)-2-[(2S)-4-cyclohexyl-2-{[(2S)-1-(4-fluorobenzoyl)pyrrolidin-2-yl]formamido}butanamido]-N-[(1S)-1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-2,2,2-trifluoro-1-methylethyl]-4-methylpentanamide

| Cpd | 1 | 2 | 3 | 4 | 5 |
|---|---|---|---|---|---|
| 21362 | Fmoc- | Fmoc- | Fmoc- | Fmoc- | Fmoc-alpha-Tfm- |
| | Aib-OH | Aib-OH | Leu-OH | Leu-OH | L-Ala-OH |
| | 6 | 7 | 8 | 9 | 10 |
| | Fmoc- | Fmoc-L- | Fmoc- | 4-Fluorobenzoic | 1-[(dimethyl- |
| | Leu-OH | homocyclohexyl | Pro-OH | acid | amino)methyl] |
| | | alanine | | | cyclobutan- |
| | | | | | 1-amine |

| | Molecular | Experimentally |
|---|---|---|
| Cpd | mass | measured mass |
| 21362 | 1166.53 | 1166.87 |
| [M + H—CF₃]+ | | |

| Cpd | R1 | R2 | R3 |
|---|---|---|---|
| 21362 | 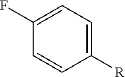 | 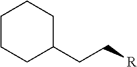 | 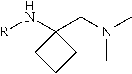 |

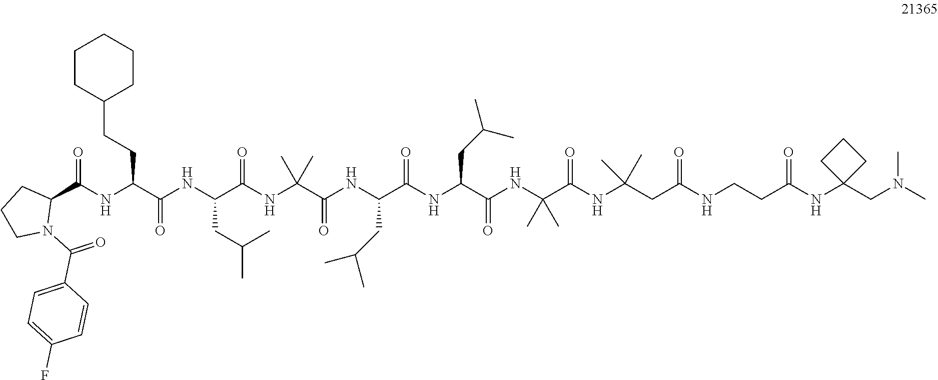 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)CC(=O)NCCC(=O)NC1(CN(C)C)CCC1]

### (2S)-2-[(2S)-4-cyclohexyl-2-{[(2S)-1-(4-fluorobenzoyl)pyrrolidin-2-yl]formamido}butan-amido]-N-(1-{[(1S)-1-{[(1S)-1-({1-[(1-{[2-({1-[(dimethylamino)methyl]cyclobutyl}carbamoyl)ethyl]carbamoyl}-2-methylpropan-2-yl)carbamoyl]-1-methylethyl}carbamoyl)-3-methylbutyl]carbamoyl}-3-methylbutyl]carbamoyl}-1-methylethyl)-4-methylpentanamide

| Cpd | 1 | 2 | 3 | 4 | 5 |
|---|---|---|---|---|---|
| 21365 | Fmoc-3- | Fmoc- | Fmoc- | Fmoc- | Fmoc- |
| | amino-3- | Aib-OH | Leu-OH | Leu-OH | Aib-OH |
| | methyl- | | | | |
| | butyric acid | | | | |
| | 6 | 7 | 8 | 9 | 10 |
| | Fmoc- | Fmoc-L- | Fmoc- | 4- | 1-[(dimethyl- |
| | Leu-OH | homocyclohexyl- | Pro-OH | Fluorobenzoic | amino)methyl] |
| | | alanine | | acid | cyclobutan- |
| | | | | | 1-amine |

| | Molecular | Experimentally |
|---|---|---|
| Cpd | mass | measured mass |
| 21365 | 1194.59 | 1194.92 |
| [M + H]+ | | |

| Cpd | R₁ | R₂ | R₃ |
|---|---|---|---|
| 21365 | 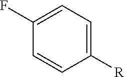 | 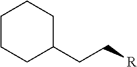 | 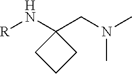 |

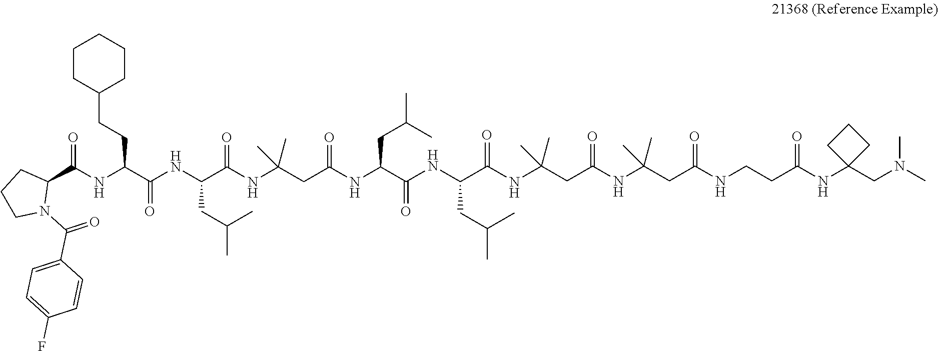 [SMILES: CC(C)C[C@H](NC(=O)CC(C)(C)NC(=O)[C@H](CC(C)C)NC(=O)[C@H](CCC1CCCCC1)NC(=O)[C@@H]1CCCN1C(=O)c1ccc(F)cc1)C(=O)N[C@@H](CC(C)C)C(=O)NC(C)(C)CC(=O)NC(C)(C)CC(=O)NCCC(=O)NC1(CN(C)C)CCC1]

### (2S)-2-[(2S)-4-cyclohexyl-2-{[(2S)-1-(4-fluorobenzoyl)pyrrolidin-2-yl]formamido}butan-amido]-N-(1-{[(1S)-1-{[(1S)-1-({1-[(1-{[2-({1-[(dimethylamino)methyl]cyclobutyl}carba-moyl)ethyl]carbamoyl}-2-methylpropan-2-yl)carbamoyl]-2-methylpropan-2-yl}carbamoyl)-3-methylbutyl]carbamoyl}-3-methylbutyl]carbamoyl}-2-methylpropan-2-yl)-4-methylpentanamide

| Cpd | 1 | 2 | 3 | 4 | 5 |
|---|---|---|---|---|---|
| 21368 | Fmoc-3- | Fmoc-3- | Fmoc- | Fmoc- | Fmoc-3- |
| | amino-3- | amino-3- | Leu-OH | Leu-OH | amino-3- |
| | methyl- | methyl- | | | methyl- |
| | butyric acid | butyric acid | | | butyric acid |
| | 6 | 7 | 8 | 9 | 10 |
| | Fmoc- | Fmoc-L- | Fmoc- | 4- | 1-[(dimethyl- |
| | Leu-OH | homocyclohexyl- | Pro-OH | Fluorobenzoic | amino)methyl] |
| | | alanine | | acid | cyclobutan- |
| | | | | | 1-amine |

| | Molecular | Experimentally |
|---|---|---|
| Cpd | mass | measured mass |
| 21368 | 1223.65 | 1223.07 |
| [M + H]+ | | |

| Cpd | R₁ | R₂ | R₃ |
|---|---|---|---|
| 21368 | 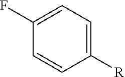 | 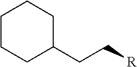 | 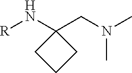 |

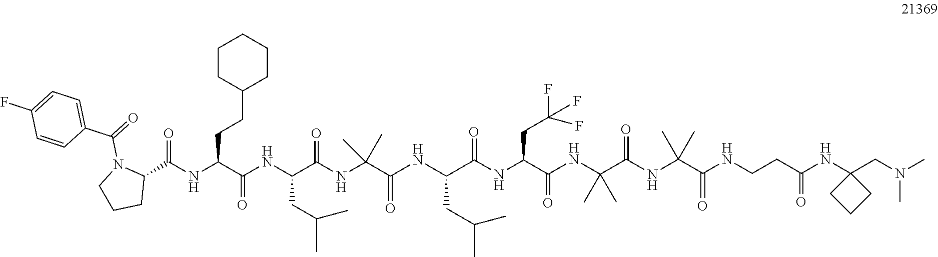 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(F)(F)F)C(=O)NC(C)(C)C(=O)NC(C)(C)C(=O)NCCC(=O)NC1(CN(C)C)CCC1]

### (2S)-2-[(2S)-4-cyclohexyl-2-{[(2S)-1-(4-fluorobenzoyl)-pyrrolidin-2-yl]formamido}butan-amido]-N-(1-{[(1S)-1-{[(1S)-1-({1-[(1-{[2-({1-[(dimethylamino)methyl]cyclobutyl}carba-moyl)ethyl]carbamoyl}-1-methylethyl)carbamoyl]-1-methylethyl}carbamoyl)-3,3,3-trifluoropropyl]carbamoyl}-3-methylbutyl]carbamoyl}-1-methylethyl)-4-methylpentanamide

| Cpd | 1 | 2 | 3 | 4 | 5 |
|---|---|---|---|---|---|
| 21369 | Fmoc- | Fmoc- | Fmoc-L- | Fmoc- | Fmoc- |
| | Aib-OH | Aib-OH | TfAbu-OH | Leu-OH | Aib-OH |
| | 6 | 7 | 8 | 9 | 10 |
| | Fmoc- | Fmoc-L- | Fmoc- | 4-Fluorobenzoic | 1-[(dimethyl- |
| | Leu-OH | homocyclohexyl | Pro-OH | acid | amino)methyl] |
| | | alanine | | | cyclobutan- |
| | | | | | 1-amine |

| | Molecular | Experimentally |
|---|---|---|
| Cpd | mass | measured mass |
| 21369 | 1206.48 | 1206.90 |
| [M]+ | | |

| Cpd | R1 | R2 | R3 |
|---|---|---|---|
| 21369 | 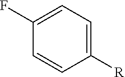 | 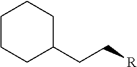 | 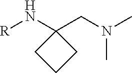 |

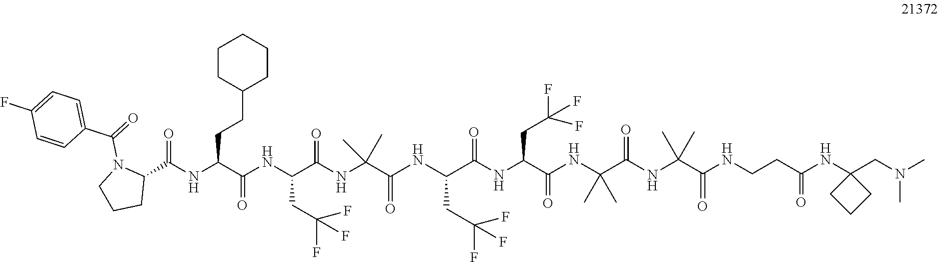 [SMILES: CN(C)CC1(NC(=O)CCNC(=O)C(C)(C)NC(=O)C(C)(C)NC(=O)[C@H](CC(F)(F)F)NC(=O)[C@H](CC(F)(F)F)NC(=O)C(C)(C)NC(=O)[C@H](CC(F)(F)F)NC(=O)[C@H](CCC2CCCCC2)NC(=O)[C@@H]2CCCN2C(=O)c2ccc(F)cc2)CCC1]

### (2S)-4-cyclohexyl-N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carba-moyl)-3,3,3-trifluoropropyl]carbamoyl}-3,3,3-trifluoropropyl]carbamoyl}-1-methylethyl) carbamoyl]-3,3,3-trifluoropropyl]-2-{[(2S)-1-(4-fluorobenzoyl)pyrrolidin-2-yl]formamido}butanamide

| Cpd | 1 | 2 | 3 | 4 | 5 |
|---|---|---|---|---|---|
| 21372 | Fmoc- | Fmoc- | Fmoc-L- | Fmoc-L- | Fmoc- |
| | Aib-OH | Aib-OH | TfAbu-OH | TfAbu-OH | Aib-OH |
| | 6 | 7 | 8 | 9 | 10 |
| | Fmoc-L- | Fmoc-L- | Fmoc- | 4-Fluorobenzoic | 1-[(dimethyl- |
| | TfAbu-OH | homocyclohexyl | Pro-OH | acid | amino)methyl] |
| | | alanine | | | cyclobutan- |
| | | | | | 1-amine |

| | Molecular | Experimentally |
|---|---|---|
| Cpd | mass | measured mass |
| 21372 | 1258.32 | 1258.63 |
| [M]+ | | |

| Cpd | R1 | R2 | R3 |
|---|---|---|---|
| 21372 | 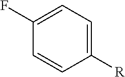 | 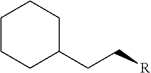 | 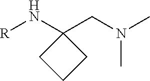 |

### Example 4

### Skin Permeation Studies

Skin permeating experiments were done with pig's skin using Franz Diffusion Cells (FDCs) using exemplarily the very preferred compound of the present invention 6027.

### Chemicals

Acetonitrile (ACN), formic acid (FA), Ethanol (EtOH) and dimethyl sulfoxide (DMSO) were all of HPLC grade and were together with Purified water was produced by reverse osmosis by Arium® pro ultrapure water system from Satorius Stedim Biotech GmbH (Göttingen, Germany).

### Skin Preparation

Skin from pig ears is widely used and accepted for skin permeation studies. Pig ears from freshly slaughtered pigs, not older than 5 months and of both sexes, were obtained from the slaughterhouse Bell Schweiz AG (Basel, Switzerland). The ears were washed with tap water and the hairs were removed with an electric clipper. The skin was separated from the underlying cartilage from the backside of the ear by using a scalpel. After washing the skin again, it was stored in aluminium foil at −20° C. for no longer than one month.

### Permeation Experiment

After thawing the frozen skin, it was cut into adequate circles and the skin thickness was measured with a calliper. The skin was mounted into the FDC, tightly sealed with Teflon band and fixed in special designed holder. Then the acceptor compartment was equipped with a magnetic stirrer, filled with placebo formulation and equilibrated for one hour. To check the barrier integrity and to be sure that there are no invisible damages from skin preparation TEWL (transepidermal water loss) was measured. The FDCs were then placed in the water bath +32° C.±1 over a magnetic stirrer (500 rpm). Due to differences of each FDC, the skin application area for drug permeation was measured and calculated for each cell in advance. Consequently, the amount of 6027 for the donor compartment had to be adjusted for the infinite-dose experiment to 284.1 μL/cm².

At predefined time points 50 μL samples were taken from the acceptor compartment. The missing volume was replaced with the corresponding placebo formulation.

At the end of the permeation experiment and before reassembling the FDCs, the donor compartments were washed out with 10 mL ACN:H₂O (55:45).

### Skin Extraction

To extract 6027 from the skin, the frozen skin was cut into small pieces and grinded by a cryogenic grinder Freezer/Mill® from Spex SamplePrep (Metuchen, USA). The setting was 10 min precooling and a rate of 10 CPS for 4 cycles with 2 minutes. The resulting powder was resuspended in 2 mL ACN:H₂O (55:45). After being vortex, the mixtures were ultrasonicated with Branson Sonifier 250 (model: 101-cx03-197) from Branson Ultrasonics Corporation (Danbury, USA). The setting for ultrasonification was output control 2 and duty cycle 30% for 60 seconds. After shaking the samples with a horizontal shaker at 37° C. for 3 minutes, the skin powder was separated by centrifugation until the supernatant appeared clear. The extraction procedure was repeated two more times.

### High Performance Liquid Chromatography Mass Spectroscopy (HPLC-MS)

The concentrations of 6027 were determined by using HPLC-MS from Agilent Technologies. The system was equipped with Infinity LAB LC/MSD XT G6135B, a degasser G1379, an isocratic pump G1310A, an autosampler G1329A with thermostat G1330B, a column oven G1316 and a C18 reversed phase column Zobrax SB—C18 Narrow Bore 2.1×150 mm 5 Micron from Agilent.

The mobile phase was a mixture of ACN:H₂O:FA (55:45:0.1) [v/v]. The flow rate was set to 0.5 mL/min, the temperature in the autosampler was set to 25° C. and the column temperature was 50° C. The injection volume was 30 μL and the total runtime was 30 min. All samples were diluted with a mixture of ACN:H₂O (55:45) to be in the calibration range. Further conditions and details such as skin thickness are listed in Table 4.

TABLE 4
Quantification of 6027 in full thickness pigs skin using Franz cells
after 48 hours. Three replicates were done, and analysis was by
HPLC-MS after skin extraction. Data shown in % of total 6027.

| | volume | | volume | API starting |
|---|---|---|---|---|
| | acceptor | medium | donor | amount |
| medium acceptor | [ml] | donor | [ml] | [mg] |
| PBS + 2% Volpo | 6 | 0.1% 6027 | 0.5 | 0.5 |
| (Brij O20, | | in DMSO | | |
| Polyalkylen- | | | | |
| glycolether) | | | | |

| skin | Total | | | Skin |
|---|---|---|---|---|
| thickness | recovery | Acceptor | Donor | extraction |
| [mm] | (%) | (%) | (%) | (%) |
| 1.4 | 95.2 | 0.2 | 79 | 16 |

A substantial skin penetration of 16% in FDC has been shown for 6027. This was in particular surprising since 6027, as well as the other compounds of formula (I), has not only a molecular weight above 1000 Da, but, moreover 6027 has a distribution coefficient octanol/water log D of 4.56 and a low water solubility of 0.022 mg/ml.

### Example 5

### In Vitro Anti-Proliferative Effect Against Cutaneous Squamous Carcinoma Cells

Cutaneous squamous cell carcinoma of the skin is a common form of skin cancer that develops in the squamous cells that make up the middle and outer layers of the skin. Cutaneous squamous cell carcinoma of the skin is usually not life-threatening, though it can be aggressive. Untreated, squamous cell carcinoma of the skin can grow large or spread to other parts of your body, causing serious complications. Most squamous cell carcinomas of the skin result from prolonged exposure to ultraviolet (UV) radiation, either from sunlight or from tanning beds or lamps. Avoiding UV light helps reduce your risk of squamous cell carcinoma of the skin and other forms of skin cancer. Squamous cells are found in many places in the body, and squamous cell carcinoma can occur anywhere squamous cells are found. Squamous cell carcinoma of the skin, and cutaneous squamous cell carcinoma (cSCC), respectively, refers to cancer that forms in the squamous cells found in the skin.

Several representative and preferred inventive compounds have been tested and showed activities against cutaneous squamous cell carcinoma cell line SCC12 in vitro.

Methodology. The cutaneous squamous cell carcinoma cell culture SCC12 was cultivated in keratinocyte-SFM media (KSFM, Cat #17005042, Gibco) supplemented with human recombinant epidermal growth factor 1-53 (EGF 1-53) and bovine pituitary extract (BFE). For the library screening, cells were distributed to 96-well plates in a cell density of 7,500 cells in 90 μl/well and let to adhere over night at 37° C. and 5% CO₂ in a cell culture incubator. Serial dilution of compounds in DMSO to 1000× was performed (10 mM, 5 mM, 1 mM, 500 μM, 250 μM, 100 μM, 10 μM, 1 μM). 2 μl of these dilutions were further diluted in 198 μl keratinocyte growth medium with supplements, mixed well and 10 μl were further dispensed in triplicates to the 96-well plates containing SCC cells in 90 μl. These dilutions resulted in a final compound concentration of 10 μM, 5 μM, 1 μM, 500 nM, 250 nM, 100 nM, 10 nM, 1 nM on cells.

Plates were incubated for additional 72 hours and cell culture medium was replaced to medium containing 0.1% of Resazurin-sodium salt stock solution (stock: 0.015 mg/ml, in PBS, Cat #R7017, Sigma-Aldrich). Cell culture plates were incubated for 2-3 hours in cell culture incubator at 37° C. and 5% CO₂. With a fluorescence-based plate reader (Infinite M200 Pro, Tecan, Switzerland) using excitations between 530-560 nM and emissions at 590 nM the cell-culture plates were readout. The calculations and graphs are performed with GraphPad Prism program. Vehicle treated wells were set to 100% viability.

TABLE 5
Half maximal inhibitory concentrations (IC50) of inventive compounds
in cutaneous squamous cell carcinoma cell line SCC12.

| Compound | IC50 (nM) |
|---|---|
| 6025 | 396 |
| 6027 | 118 |
| 6253 | 140 |
| 6328 | 1656 |
| 6781 | 6849 |
| 7058 | 717 |
| 7059 | 500 |
| 7824 | 375 |
| 7844 | 546 |
| 7848 | 182 |
| 7850 | 1042 |
| 8341 | 1019 |
| 8342 | 584 |
| 8343 | 241 |
| 21164 | 712 |
| 21189 | >10000 |
| (Reference | |
| Example) | |
| 21287 | 595 |
| 21314 | 324 |
| 21315 | 3104 |
| 21359 | 360 |
| 21362 | 717 |
| 21365 | 511 |
| 21368 | >10000 |
| (Reference | |
| Example) | |
| 21369 | 167 |
| 21372 | 442 |

Results. The tested preferred inventive showed activities and IC50 values in the nM range against cutaneous squamous cell carcinoma SCC12 in vitro confirming its usefulness as drugs for the treatment of skin cancers in particular cutaneous squamous skin cancer.

### Example 6

### In Vivo Anti-Proliferative Assay

In vitro anti-proliferative effects against 6 skin cancer derived cells including such from cutaneous melanoma (M150672, M150507, M010817), and cutaneous squamous cell carcinoma cell lines (SCC13 and A431_SCC) and cutaneous T cell lymphoma (MyLa_CTCL) have been tested for the preferred inventive compound 6027. Likewise, anti-proliferative effects against healthy skin dermal fibroblasts were established.

Methodology. For the library screening cells were distributed to 96-well plates (cell densities (cells/well): M150672 (2000), M150507 (1500), M010817 (1500), M130107 (2000), SCC13 (11000), A431_SCC (1500)), MyLa_CTCL (10′000) in 90 μl/well and let to adhere over night at 37° C. and 5% CO₂ in a cell culture incubator.

For the maintenance of healthy human fibroblast cell culture Dulbecco's Modified Eagle Medium (DMEM, Cat #11966025, Gibco) supplemented with 10% heat-inactivated fetal bovine serum (Cat #S006420H01, BioWest) was used. For the maintenance of melanoma cell cultures RPMI-1640 (Cat #R0883, Gibco) supplemented with L-glutamine (Cat #25030-081, Gibco), 1 mM sodium pyruvate (Cat #S8636, Sigma-Aldrich), 10% heat-inactivated fetal bovine serum (Cat #S006420H01, BioWest) and 1% Penicillin-Streptomycin (Cat #15140122, Gibco) was used. The MyLa_CTCL cell cultures were cultivated in RPMI 1640 Medium (R0883) supplemented with 2 mM L-Glutamine (G7513), 10 U/ml IL-2 (H7041), 10 U/ml IL-4 (H7291) and 10% Human AB Serum (H4522). The cutaneous squamous cell carcinoma cell cultures A431 and SCC13 were cultivated in Dulbecco's Modified Eagle Medium (DMEM, Cat #11966025, Gibco) supplemented with 10% heat-inactivated fetal bovine serum (Cat #S006420H01, BioWest).

To determine the half maximal inhibitory concentrations (IC50's) serial dilution of compounds in DMSO to 1000× was performed (10 mM, 5 mM, 1 mM, 500 μM, 250 μM, 100 μM, 10 μM, 1 μM). 2 μl of these dilutions were further diluted in 198 μl growth medium with supplements, mixed well and 10 μl were further dispensed in triplicates to the 96-well plates containing cells in 90 μl. These dilutions resulted in a final compound concentration of 10 μM, 5 μM, 1 μM, 500 nM, 250 nM, 100 nM, 10 nM, 1 nM.

For each drug condition and cell culture (for each row of the triplicate) a well with only RPMI-1640 complete/KSFM/DMEM media and one with media and DMSO were made as controls. DMSO was never at a higher concentration than 0.2% v/v. Triplicates were seeded for each condition. On the day of the assay, the medium was exchanged with cell culture media containing 0.1% of Resazurin-sodium salt stock solution (stock: 0.015 mg/ml, in PBS, Cat #R7017, Sigma-Aldrich). Cell culture plates were incubated for 2-3 hours in cell culture incubator at 37° C. and 5% CO₂. With a fluorescence-based plate reader (Infinite M200 Pro, Tecan, Switzerland) using excitations between 530-560 nM and emissions at 590 nM the cell-culture plates were readout. The calculations and graphs are performed with GraphPad Prism program. Vehicle treated wells were set to 100% viability.

TABLE 6
Cytotoxic potency of 6027 shown as IC50 values in nM
tested against healthy skin dermal fibroblasts, and 5 skin
cancer derived cells including such from cutaneous squamous
cell carcinoma cell lines (SCC13, and A431_SCC) as well as
such from cutaneous melanoma (M010817, M150672, M150507)
and cutaneous T cell lymphoma (MyLa_CTCL).

| | Fibroblast | M010817 | M130107 | M150672 | M150507 |
|---|---|---|---|---|---|
| 6027 | 3028 | 60 | 169 | 122 | 56 |

| | SCC13 | A431 | MyLa_CTCL |
|---|---|---|---|
| 6027 | 191 | 70 | 451 |

Results. 6027 showed IC50 values in the nM range for the various skin cancer cell lines. Against healthy skin dermal fibroblasts the cytotoxicity was less pronounced. This suggests a selectivity between skin cancer derived cells and healthy skin cells confirming the potential therapeutic window of the inventive compounds and in particular of 6027 for development as a drug to treat skin cancer.

### Example 7

### In Vitro Anti-Proliferative and Pro-Apoptotic Activity Against B16-F10 Mouse Melanoma Cells in C57BL/6 Mice Bearing B16F10 Xenografts

The murine B16 melanoma model is the most commonly used metastatic melanoma model for preclinical studies. The syngeneic B16-F10 model was used to evaluate responses in tumour development after dermal application of 6027. Leucinostatin A, a known natural peptide was initially tested similarly.

In vivo intradermal implant of B16-F10 cells in C57BL/6 mice results in aggressively growing tumours. Our growth studies show efficient growth kinetics following inoculation. Control animals stay on study for 10-15 days before they reach euthanasia criteria of excessive tumour burden or ulceration. This results in a model which can facilitate up to a two-week dosing window for test agents to elicit their anti-tumour activity.

To determine whether C57BL/6 mice bearing B16-F10 xenografts is a suitable model to study the potential in vivo efficacy of dermally applied 6027 an in vitro test for half maximum inhibition (IC50) was performed on B16F10 melanoma cells. Leucinostatin A was tested likewise.

In vitro cell viability assay methodology. For the maintenance of B16-F10 mouse melanoma cell culture RPMI-1640 (Cat #R0883, Gibco) supplemented with L-glutamine (Cat #25030-081, Gibco), 1 mM sodium pyruvate (Cat #S8636, Sigma-Aldrich), 10% heat-inactivated fetal bovine serum (Cat #S006420H01, BioWest) and 1% Penicillin-Streptomycin (Cat #15140122, Gibco) was used.

Half maximal inhibitory concentration (IC50) of the compounds was determined by seeding of cell culture into 96 well-plates (Cat #353072, Corning) and treated the day after with dose-escalating concentrations of the compounds (from 0.001 μM to 10 μM in RPMI-1640 complete) for 72 hours. 1000 cells were seeded per well. For each drug condition and cell culture (for each row of the triplicate) a well with only RPMI-1640 complete media and one with media and DMSO were made as controls. DMSO was never at a higher concentration than 0.2% v/v. Triplicates were seeded for each condition. On the day of the assay, the medium was exchanged with cell culture media containing 0.1% of Resazurin-sodium salt stock solution (stock: 0.015 mg/ml, in PBS, Cat #R7017, Sigma-Aldrich). Cell culture plates were incubated for 2-3 hours in cell culture incubator at 37° C. and 5% CO₂. With a fluorescence-based plate reader (Infinite M200 Pro, Tecan, Switzerland) using excitations between 530-560 nM and emissions at 590 nM the cell-culture plates were readout. The calculations and graphs are performed with GraphPad Prism program. Vehicle treated wells were set to 100% viability. 6027 had an IC50 of 208 nM, leucinostatin A had an IC50 of 161 nM.

In vivo efficacy test methodology. C57Bl/6 mice (Janvier Labs); females; 6-8 weeks old at beginning of the treatment. The tumour was induced through intradermal inoculation of 1×105 cells per mouse intradermally in the skin of the neck. A total of 24 mice were used (n=8 for vehicle, n=8 for 5% 6027, n=8 for leucinostatin A 0.2%). Test item was applied starting on day 1 after inoculation of the tumour cells. The test items of 20 μl of 5% 6027 and of 0.2% Leucinostatin A were applied dermally with a pipette once a day every day for up to 21 days to the intact skin area of about 1 cm² on the site were the B16-F10 cells had been injected. If ulceration occurred then the animal was removed from the test and sacrificed.

The read out was tumour volume measured three times a week expressed as tumour volume (in cm³). Additionally, tumour samples were taken for histological analysis (Tumour FFPE) (2 mice per group).

Results. Dermal treatment with 20 μl of 5% 6027 on the area in which the tumours had been inoculated was well tolerated. No skin irritations or other side effects were observed. B16F10 tumours grew progressively in the neck region of mice after intradermal inoculation. At lowest test concentration of 0.2% leucinostatin A showed strong skin irritation with destruction of the epidermis. Therefore, treatment with 0.200 leucinostatin A was not tolerated; and for ethical reasons the treatment regimen could not be continued longer than 7 days. The application site was left untreated thereafter until the end of the experiment on day 16. The animals in this group showed no significant reduction of tumour volume throughout the duration of the test.

TABLE 7
Tumour volume of from C57BL/6 mice bearing intradermal B16F10 xenografts which
had received daily treatment of either 20 μl of 5% 6027 (group B: n =
8) or vehicle control (group A: n = 8)) on the skin area of the intradermal inoculation
(1 cm²). Tumour volume was measured 3 times a week with a calipper and expressed as volume
in cm³. The control groups mean tumour volume was normalized to 1 for the control
group (RTV) and the ratio of mean tumour volume of treated over control is indicated
(T/C %). Tumours were implanted on day 0 and treatment with 6027 and vehicle control
was started on day 1. Data are shown through day 11.

Meas.

| | | | 1. | 2. | 3. | 4. | 5. | 6. | 7. |
|---|---|---|---|---|---|---|---|---|---|
| | | | Meas. | Meas. | Meas. | Meas. | Meas. | Meas. | Meas. |
| Group | Day: | | 1 | 2 | 4 | 7 | 9 | 10 | 11 |
| A | (n) | | 8 | 8 | 8 | 8 | 8 | 8 | 8 |
| Placebo | | | | | | | | | |
| | Tumour | Median | 0.001 | 0.001 | 0.001 | 0.011 | 0.033 | 0.066 | 0.107 |
| | Volume | | | | | | | | |
| | [cm³] | Mean | 0.001 | 0.001 | 0.001 | 0.012 | 0.044 | 0.086 | 0.136 |
| | | [S.D.] | 0.00 | 0.00 | 0.00 | 0.01 | 0.04 | 0.06 | 0.10 |
| | RTV | Median | 1.0 | 1.0 | 1.0 | 10.5 | 32.5 | 66.0 | 106.5 |
| | | Mean | 1.0 | 1.0 | 1.0 | 12.4 | 44.3 | 85.8 | 135.5 |

| | | | Meas. | | | | | | |
|---|---|---|---|---|---|---|---|---|---|
| | | | Gr. B | Gr. B | Gr. B | Gr. B | Gr. B | Gr. B | Gr. B |
| | | | M1 | M2 | M3 | M4 | M5 | M6 | M7 |
| B | (n) | | 8 | 8 | 8 | 8 | 8 | 8 | 8 |
| 5% 6027 | | | | | | | | | |
| | Tumour | Median | 0.001 | 0.001 | 0.001 | 0.009 | 0.026 | 0.026 | 0.031 |
| | Volume | | | | | | | | |
| | [cm³] | Mean | 0.001 | 0.001 | 0.001 | 0.009 | 0.028 | 0.030 | 0.045 |
| | | [S.D.] | 0.00 | 0.00 | 0.00 | 0.00 | 0.01 | 0.01 | 0.03 |
| | RTV | Median | 1.0 | 1.0 | 1.0 | 9.0 | 25.5 | 25.5 | 31.0 |
| | | Mean | 1.0 | 1.0 | 1.0 | 8.8 | 28.4 | 29.9 | 44.6 |
| | T/C [%] | | 100 | 100 | 100 | 71 | 64 | 35 | 33 |

Histopathology. After sacrifice non ulcerated tumours were sampled and paraffin blocks were prepared for immunohistochemistry stainings. Ki-67 staining was used as an immunohistochemical cellular marker for proliferation to visualize regions of the slice with proliferating cells. During interphase, the Ki-67 antigen can be exclusively detected within the cell nucleus, whereas in mitosis most of the protein is relocated to the surface of the chromosomes. Ki-67 protein is present during all active phases of the cell cycle (G1, S, G2 and mitosis), but is absent in resting (quiescent) cells (G0). Cellular content of Ki-67 protein markedly increases during cell progression through S phase of the cell cycle.

TUNEL staining was used to determine areas of the tumour in which apoptosis was observed. Terminal deoxynucleotidyl transferase dUTP nick end labeling (TUNEL) staining is a method for detecting DNA fragmentation by labeling the 3′-hydroxyl termini in DNA double-stranded breaks generated during apoptosis.

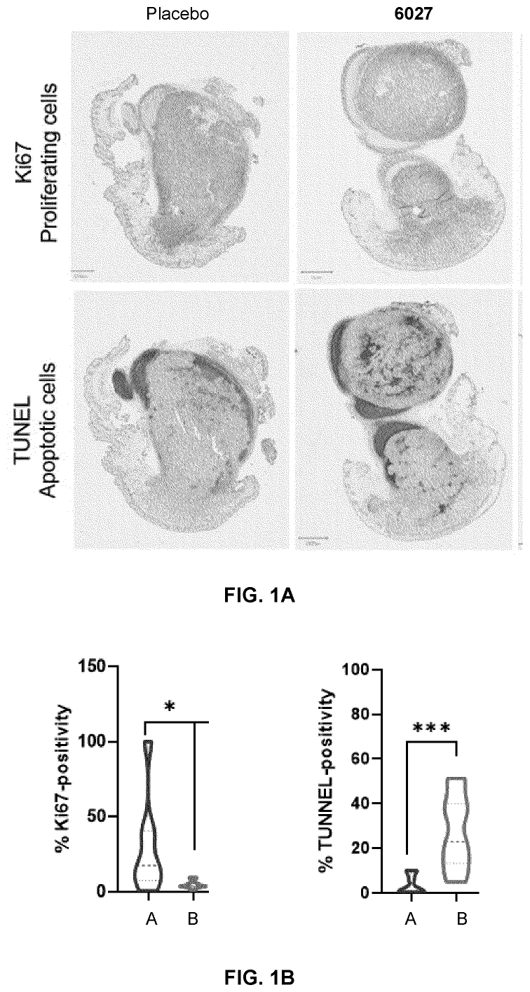

Results. In tumour slices from C57BL/6 mice bearing B16F10 xenografts which had received daily treatment of 20 μl of 6027, the tumours showed a significant decrease in proliferative cells and a significant increase of apoptotic cells for 6027 as compared to the tumours from vehicle treated controls (Two-Way ANOVA, *p<0.01; ***p<0.0001) as shown in FIG. 1A and FIG. 1B.

Thus, the topical treatment of the inventive compound 6027 shows significant reduction of tumour growth against B16-F10 mouse melanoma cells in C57BL/6 mice bearing B16F10 xenografts the most used metastatic melanoma model for preclinical studies. Strong proapoptotic potential is shown in histological studies of the tumours compared to control. It is of note that leucinostatin A, a known natural peptide, while having a similar in vitro cytotoxicity against B16-F10 cells, is not suitable for dermal application as it is 6027 because it has a strong skin irritating effect down to concentrations of 0.2% which is 25 times lower than the 5% at which 6027 did not show skin irritations.

### Example 8

### Inhibitory Effects of 6027 on Ex Vivo Patient-Derived Basal Cell Carcinoma (BCC)

Tumour material was harvested from an infiltrative basal cell carcinoma (BCC) from a 91 year old patient's nose.

Fresh tumour material of said patient was cut into similar sized pieces, put on cell culture inserts (0.4 μM, Millicell cell culture; Millipore, Cat #PICM03050) and incubated with 1 ml of co-culture media (3 parts DMEM (Gibco, Cat. #41966052), 1 part Ham's F-12 Nutrient Mix (Gibco, Cat. #11765054), 10% Fetal Bovine serum heat inactivated (Gibco, Cat. #1050), 0.1 mg/ml of Normocin™ (Invivogen, Cat. #ant-nr-1), 21.8 g/mL of Adenin (Sigma-Aldrich, Cat. #A2786), 5.45 μg/mL of Apotransferrin (Sigma-Aldrich, Cat. #T1147), 2.18 nM of Triiodothyronine (Sigma-Aldrich, Cat. #T6397), 0.44 g/mL of Hydrocortisone (Sigma-Aldrich, Cat. #H0888), 0.11 nM of Cholera toxin (Sigma-Aldrich, Cat. #C8052), 5.50 g/mL of Insulin (Sigma-Aldrich, Cat. #16634) and 0.01 g/mL of Epidermal growth factor (Sigma-Aldrich, Cat. #E4127)) from below for 24 hours at 37° C. and 5% CO₂ to let the tissue piece recover. After 24 hours, the cell culture media was replaced with cell culture media containing drug conditions and incubated for another 4 days at 37° C. and 5% CO₂. One made sure that tissue piece is covered with a drop of media containing drugs (day 0 and day 2). After 4 days of treatment, tissue was fixed in 4% buffered formaldehyde followed by immunohistochemistry stainings for histopathological examination and comparison of treatment conditions to untreated controls (DMSO).

Immunohistochemistry of ex vivo tumour pieces: Tumour slices were embedded into paraffin blocks. Consecutive cuts were stained with antibodies of interest. Following antibodies were used for stainings: Ki67 (Dako, clone MIB-1, Cat #M7240, Dilution 1:50, Antigen Retrieval: Leica Bond ER2: 30 min at 95° C.). BERep4 (Dako Agilent, M0804, Dilution 1:300, Antigen Retrieval Ph9) and HE (Hämalaun nach Mayer, Artechemis, Cat #T.865.3; Eosin 1% wässrig—Morphisto, Cat #10177). The slides were scanned at Vectra Polaris Slide Scanner (Akoya Biosystems) and analyzed with Qpath software.

BERep4 staining was used as an immunohistochemical cellular marker for regions of the slice with basal cell carcinoma cells. BERep4 (also Ber-EP4) is a histologic stain mainly used to aid in the diagnosis of basal cell carcinoma (BCC) and is an antibody to EpCAM (epithelial cell adhesion molecule). KI-67 staining was used as an immunohistochemical cellular marker for proliferation to visualize regions of the slice with proliferating cells. Vismodegib (20 μM), as the standard of care, was further used as a positive control.

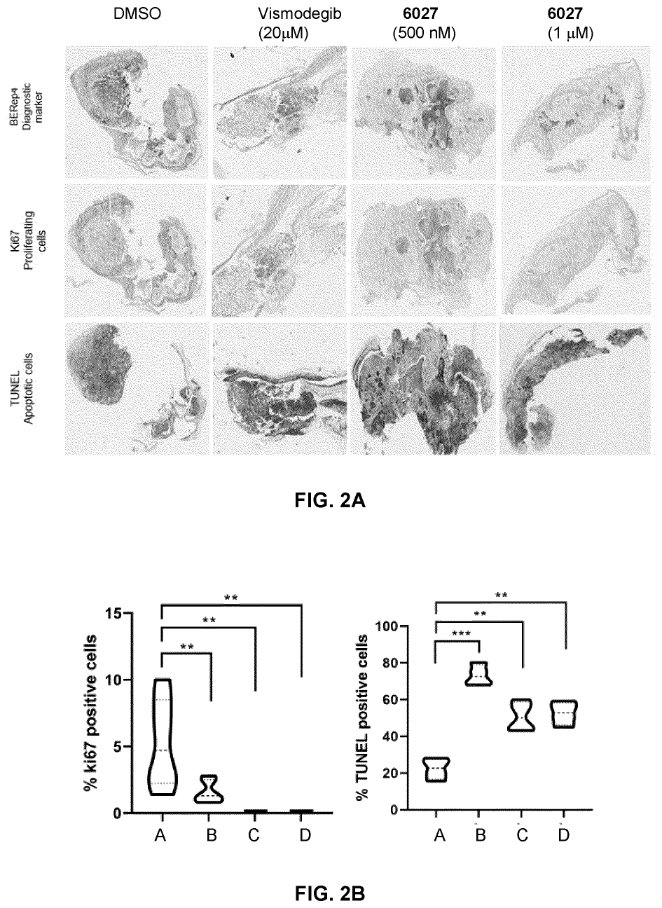

Results: Incubation of patient derived BCC tumours for 4 days showed that cell proliferation was significantly reduced in the presence of 500 nM and 1 μM of 6027 as well as Vismodegib (20 μM) compared to negative control DMSO (Two-Way ANOVA; **p<0.002). Significant increase in apoptotic cells after treatment with 6027 in 500 nM and 1p M concentration indicated by TUNEL staining (Two-Way ANOVA; ***p<0.0001; **p<0.002) as shown in FIG. 2A and FIG. 2B.

### Example 9

### Inhibitory Effects of 6027 on Ex Vivo Patient-Derived Basal Cell Carcinoma BCC

Tumour material was harvested from a patient's infiltrative basal cell carcinoma (BCC).

Fresh punch biopsy tumour material of said patient was cut into similar sized pieces, put on cell culture inserts (0.4 μM, Millicell cell culture; Millipore, Cat #PICM03050) and incubated with 1 ml of co-culture media (3 parts DMEM (Gibco, Cat. #41966052), 1 part Ham's F-12 Nutrient Mix (Gibco, Cat. #11765054), 10% Fetal Bovine serum heat inactivated (Gibco, Cat. #1050), 0.1 mg/ml of Normocin™ (Invivogen, Cat. #ant-nr-1), 21.8 g/mL of Adenin (Sigma-Aldrich, Cat. #A2786), 5.45 g/mL of Apotransferrin (Sigma-Aldrich, Cat. #T1147), 2.18 nM of Triiodothyronine (Sigma-Aldrich, Cat. #T6397), 0.44 g/mL of Hydrocortisone (Sigma-Aldrich, Cat. #H0888), 0.11 nM of Cholera toxin (Sigma-Aldrich, Cat. #C8052), 5.50 g/mL of Insulin (Sigma-Aldrich, Cat. #16634) and 0.01 g/mL of Epidermal growth factor (Sigma-Aldrich, Cat. #E4127)) from below for 24 hours at 37° C. and 5% CO₂ to let the tissue piece recover. After 24 hours, the cell culture media was replaced with cell culture media containing drug conditions and incubated for another 4 days at 37° C. and 5% CO₂. One made sure that tissue piece is covered with a drop of media containing drugs (day 0 and day 2). After 4 days of treatment, tissue was fixed in 4% buffered formaldehyde followed by immunohistochemistry stainings for histopathological examination and comparison of treatment conditions to untreated controls (DMSO).

Immunohistochemistry of ex vivo tumour pieces: Tumour slices were embedded into paraffin blocks. Consecutive cuts were stained with antibodies of interest. Following antibodies were used for stainings: Ki67 (Dako, clone MIB-1, Cat #M7240, Dilution 1:50, Antigen Retrieval: Leica Bond ER2: 30 min at 95° C.). BERep4 (Dako Agilent, M0804, Dilution 1:300, Antigen Retrieval Ph9) and HE (Hämalaun nach Mayer, Artechemis, Cat #T.865.3; Eosin 1% wässrig—Morphisto, Cat #10177). The slides were scanned at Vectra Polaris Slide Scanner (Akoya Biosystems) and analyzed with Qpath software.

BERep4 staining was used as an immunohistochemical cellular marker for regions of the slice with basal cell carcinoma cells. BERep4 (also Ber-EP4) is a histologic stain mainly used to aid in the diagnosis of basal cell carcinoma (BCC) and is an antibody to EpCAM (epithelial cell adhesion molecule). KI-67 staining was used as an immunohistochemical cellular marker for proliferation to visualize regions of the slice with proliferating cells. Vismodegib (20 μM) as the standard of care, was further used as a positive control.

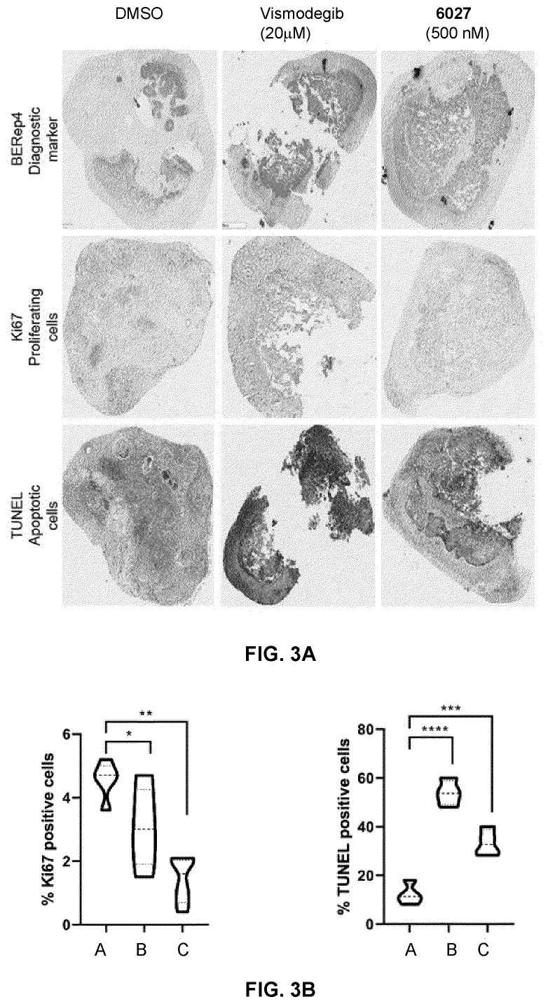

Results. Incubation of patient derived BCC tumours for 4 days showed that cell proliferation was significantly reduced in the presence of 500 nM of 6027 as well as Vismodegib (20 μM) compared to negative control DMSO (Two-Way ANOVA; *p<0.01; **p<0.002). Significant increase in apoptotic cells after treatment with 6027 (500 nM) and Vismodegib (20 μM) indicated by TUNEL positive cells (Two-Way ANOVA; ***p<0.0001; ****p<0.00002) as shown in FIG. 3A and FIG. 3B.

### Example 10

### Inhibitory Effects of 6027 on Ex Vivo Patient-Derived Basal Cell Carcinoma BCC

Tumour material was harvested from a nodular basal cell carcinoma (BCC) from a 73 year old patient's upper back.

Fresh tumour material of said patient was cut into similar sized pieces put on cell culture inserts (0.4 μM, Millicell cell culture; Millipore, Cat #PICM03050) and incubated with 1 ml of co-culture media (3 parts DMEM (Gibco, Cat. #41966052), 1 part Ham's F-12 Nutrient Mix (Gibco, Cat. #11765054), 10% Fetal Bovine serum heat inactivated (Gibco, Cat. #1050), 0.1 mg/ml of Normocin™ (Invivogen, Cat. #ant-nr-1), 21.8 g/mL of Adenin (Sigma-Aldrich, Cat. #A2786), 5.45 g/mL of Apotransferrin (Sigma-Aldrich, Cat. #T1147), 2.18 nM of Triiodothyronine (Sigma-Aldrich, Cat. #T6397), 0.44 g/mL of Hydrocortisone (Sigma-Aldrich, Cat. #H0888), 0.11 nM of Cholera toxin (Sigma-Aldrich, Cat. #C8052), 5.50 g/mL of Insulin (Sigma-Aldrich, Cat. #16634) and 0.01 g/mL of Epidermal growth factor (Sigma-Aldrich, Cat. #E4127)) from below for 24 hours at 37° C. and 5% CO₂ to let the tissue piece recover. After 24 hours the cell culture media was replaced with cell culture media containing drug conditions and incubated for another 4 days at 37° C. and 5% CO₂. One made sure that tissue piece is covered with a drop of media containing drugs (day 0 and day 2). After 4 days of treatment tissue was fixed in 4% buffered formaldehyde followed by immunohistochemistry stainings for histopathological examination and comparison of treatment conditions to untreated controls (DMSO).

Immunohistochemistry of ex vivo tumour pieces: Tumour slices were embedded into paraffin blocks. Consecutive cuts were stained with antibodies of interest. Following antibodies were used for stainings: Ki67 (Dako, clone MIB-1, Cat #M7240, Dilution 1:50, Antigen Retrieval: Leica Bond ER2: 30 min at 95° C.). BERep4 (Dako Agilent, M0804, Dilution 1:300, Antigen Retrieval Ph9) and HE (Hämalaun nach Mayer, Artechemis, Cat #T.865.3; Eosin 1% wässrig—Morphisto, Cat #10177). The slides were scanned at Vectra Polaris Slide Scanner (Akoya Biosystems) and analyzed with Qpath software.

BERep4 staining was used as an immunohistochemical cellular marker for regions of the slice with basal cell carcinoma cells. BERep4 (also Ber-EP4) is a histologic stain mainly used to aid in the diagnosis of basal cell carcinoma (BCC) and is an antibody to EpCAM (epithelial cell adhesion molecule). Ki-67 staining was used as an immunohistochemical cellular marker for proliferation to visualize regions of the slice with proliferating cells. Vismodegib (20 μM) was used as a positive control.

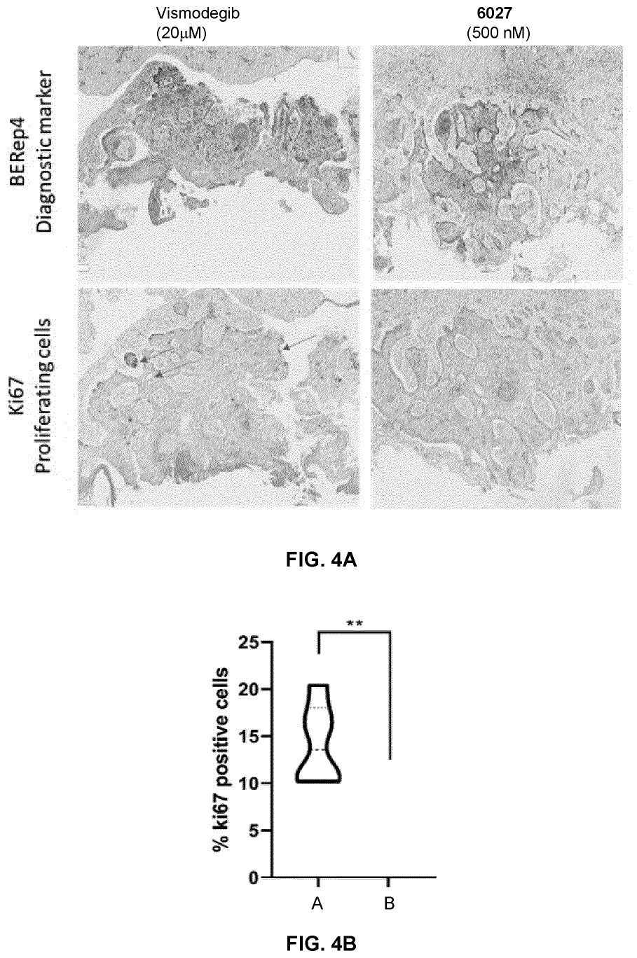

Results. Incubation of patient derived BCC tumours for 4 days showed that cell proliferation was significantly reduced in the presence of 500 nM of 6027 compared to standard of care treatment Vismodegib (20 μM) (Two-Way ANOVA; **p<0.002) as shown in FIG. 4A and FIG. 4B.

### Example 11

### Inhibitory Effects of 6027 on Ex Vivo Patient-Derived Cutaneous Squamous Cell Carcinoma cSCC

Tumour material was harvested from high grade cutaneous squamous cell carcinoma (cSCC) from a 79-year-old patient's head.

Fresh tumour material of said patient was cut into similar sized pieces put on cell culture inserts (0.4 μM, Millicell cell culture; Millipore Cat #PICM03050) and incubated with 1 ml of co-culture media (3 parts DMEM (Gibco, Cat. 41966052), 1 part Ham's F-12 Nutrient Mix (Gibco, Cat. #11765054), 10% Fetal Bovine serum heat inactivated (Gibco, Cat. #1050), 0.1 mg/ml of Normocin™ (Invivogen, Cat. #ant-nr-1), 21.8 g/mL of Adenin (Sigma-Aldrich, Cat. #A2786), 5.45 g/mL of Apotransferrin (Sigma-Aldrich, Cat. #T1147), 2.18 nM of Triiodothyronine (Sigma-Aldrich, Cat. #T6397), 0.44 g/mL of Hydrocortisone (Sigma-Aldrich, Cat. #H0888), 0.11 nM of Cholera toxin (Sigma-Aldrich, Cat. #C8052), 5.50 g/mL of Insulin (Sigma-Aldrich, Cat. #16634) and 0.01 g/mL of Epidermal growth factor (Sigma-Aldrich, Cat. #E4127)) from below for 24 hours at 37° C. and 5% CO₂ to let the tissue piece recover. After 24 hours, the cell culture media was replaced with cell culture media containing drug conditions and incubated for another 4 days at 37° C. and 5% CO₂. One made sure that tissue piece is covered with a drop of media containing drugs (day 0 and day 2). After 4 days of treatment, tissue was fixed in 4% buffered formaldehyde followed by immunohistochemistry stainings for histopathological examination and comparison of treatment conditions to untreated controls (DMSO).

Immunohistochemistry of ex vivo tumour pieces: Tumour slices were embedded into paraffin blocks. Consecutive cuts were stained with antibodies of interest. Following antibodies were used for stainings: Ki67 (Dako, clone MIB-1, Cat #M7240, Dilution 1:50, Antigen Retrieval: Leica Bond ER2: 30 min at 95° C.). BERep4 (Dako, M0804, Dilution 1:300, Antigen Retrieval Ph9) and HE (Hämalaun nach Mayer, Artechemis, Cat #T.865.3; Eosin 1% wässrig —Morphisto, Cat #10177). The slides were scanned at Vectra Polaris Slide Scanner (Akoya Biosystems) and analyzed with Qpath software.

HE staining: Hematoxylin and eosin (HE) stain is one of the principal tissue stains used in histology. HE is the combination of two histological stains: hematoxylin and eosin. The hematoxylin stains cell nuclei as purplish blue and eosin stains the extracellular matrix and cytoplasm pink with other structures taking on different shades hues and combinations of these colours to easily differentiate between the nuclear and cytoplasmic parts of a cell. Additionally, the overall patterns of coloration from the stain show the general layout and distribution of cells and provides a general overview of a tissue sample's structure and can be used as a SCC diagnostic tool due to the typical immunohistochemical characteristics of a SCC.

Ki-67 staining was used as an immunohistochemical cellular marker for proliferation to visualize regions of the slice with proliferating cells.

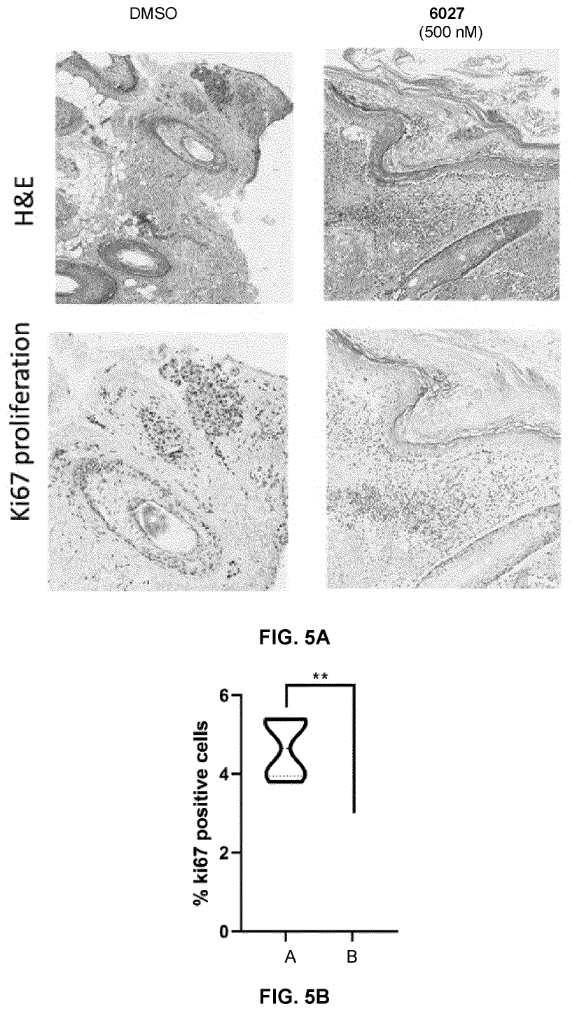

Results: Incubation of patient derived SCC tumours for 4 days showed that cell proliferation was significantly reduced in the presence of 500 nM of 6027 compared to the negative control DMSO (Two-Way ANOVA; **p<0.003) as shown in FIG. 5A and FIG. 5B.

1. A compound of formula (I) for use in a method of topical treatment of a skin cancer of a mammal, wherein said method comprises topical administration of said compound to said mammal, 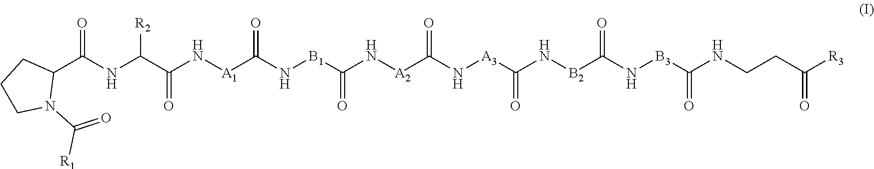
wherein
A₁ is 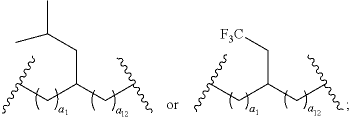
A₂ is 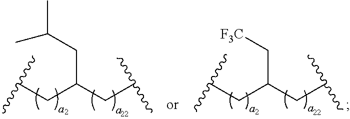
A₃ is 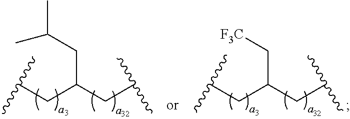
B₁ is 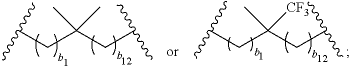
B₂ is 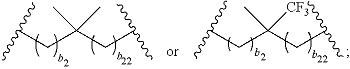
B₃ is 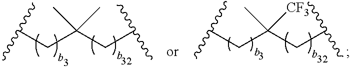
and wherein
a₁, a₁₂, a₂, a₂₂, a₃, a₃₂, b₁, b₁₂, b₂, b₂₂, b₃ and b₃₂ are independently selected from 0 and 1, wherein a₁+a₁₂, a₂+a₂₂, a₃+a₃₂, b₁+b₁₂, b₂+b₂₂, and b₃+b₃₂ are independently selected from 0 and 1, and wherein a₁+a₁₂+a₂+a₂₂+a₃+a₃₂ is 0, 1, 2 or 3, and wherein b₁+b₁₂+b₂+b₂₂+b₃+b₃₂ is 0, 1 or 2;
and wherein
R₁ is selected from carbocyclyl or heterocyclyl, each independently optionally and preferably substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl;
R₂ is selected from C₄-C₁₂alkyl, C₄-C₁₀alkoxy, C₁-C₃alkylene-cycloalkyl, C₁-C₃alkylene-aryl, C₁-C₃alkylene-heteroaryl, wherein independently in said C₁-C₃alkylene one —CH₂— moiety is optionally replaced by —CH(NH)— or —O—; and wherein said alkyl, cycloalkyl, aryl and heteroaryl are each independently optionally and preferably substituted with one or more, preferably one or two, substituents selected from C₁-C₂alkyl, C₁-C₂haloalkyl, oxo, OH, halogen, C₁-C₂alkoxy, C₆H₅ or C₆H₅ substituted with C₁-C₃alkyl or OC₁-C₃alkyl;
R₃ is 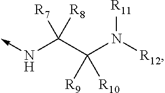
wherein
R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a carbocyclic or heterocyclic ring, preferably a carbocyclic ring, and wherein
R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a heteroaryl or a heterocyclyl, each independently optionally and preferably substituted with halogen, C₁-C₄alkyl, OR₁₃, NR₁₄R₁₅; wherein R₁₃, R₁₄, R₁₅ are independently at each occurrence H, C₁-C₄alkyl, and wherein the arrow indicates the attachment to the C(O)-moiety depicted in formula (I);
and pharmaceutically acceptable salts of said compound of formula (I).

2. The compound for use of claim 1, wherein said compound of formula (I) is a compound of formula (I*) 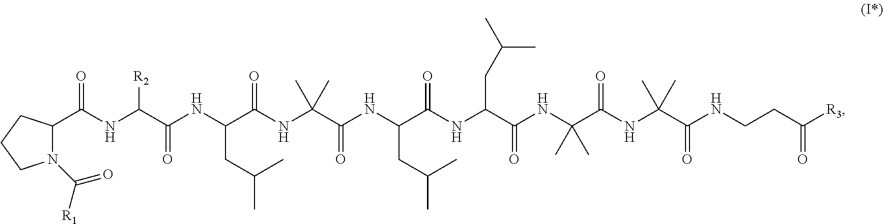
and wherein preferably said compound of formula (I) is a compound of any one of the formula (II) to (IV) 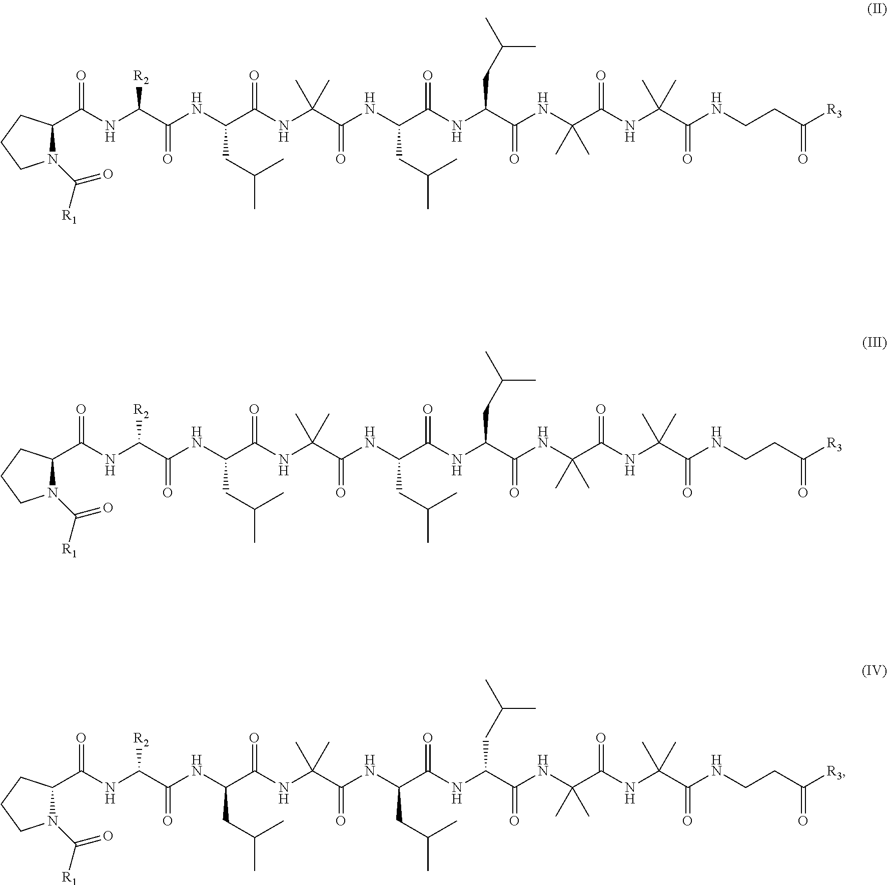
wherein further preferably said compound of formula (I) is a compound of formula (II).

3. The compound for use of claim 1 or claim 2, wherein said R₁ is selected from cycloalkyl, aryl or heteroaryl, preferably selected from cycloalkyl, monocyclic or bicyclic aryl or monocyclic or bicyclic heteroaryl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

4. The compound for use of any one of the claims 1 to 3, wherein said R₁ is selected from phenyl or a monocyclic or bicyclic heteroaryl comprising one or two heteroatoms selected from N, O and S, preferably from phenyl or oxazolyl; each independently optionally substituted with C₁-C₄alkyl, halogen, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

5. The compound for use of any one of the claims 1 to 4, wherein said R₁ is selected from the formula 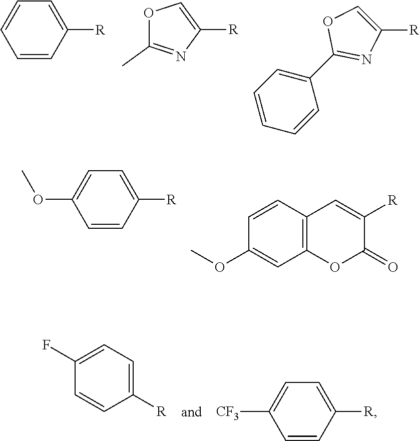
wherein R indicates the attachment to the C(O)-moiety depicted in formula (I).

6. The compound for use of any one of the claims 1 to 5, wherein said R₂ is selected from C₅-C₁₂alkyl, C₄-C₁₀alkoxy, C₁-C₃alkylene-C₅-C₆cycloalkyl, C₁-C₃alkylene-phenyl, C₁-C₃alkylene-biphenyl, C₁-C₃alkylene-(mono- or bicyclic-heteroaryl), wherein independently in said C₁-C₃alkylene one —CH₂— moiety is optionally replaced by —CH(NH)— or —O—, and wherein said phenyl, biphenyl, C₅-C₆-cycloalkyl, and mono- or bicyclic-heteroaryl are each independently optionally substituted with one or more, preferably with one or two, substituents selected from C₁-C₂alkyl, C₁-C₂haloalkyl, halogen, C₁-C₂alkoxy.

7. The compound for use of any one of the claims 1 to 6, wherein said R₂ is selected from C₅-C₁₂alkyl, C₁-C₂alkylene-C₅-C₆cycloalkyl, C₁-C₂alkylene-phenyl, C₁-C₂alkylene-biphenyl, C₁-C₂alkylene-(mono- or bicyclic-heteroaryl), wherein independently in said C₁-C₂alkylene one —CH₂— moiety is optionally replaced by —CH(NH)— or —O—, preferably by —O—, and wherein said phenyl biphenyl, C₅-C₆-cycloalkyl, and mono- or bicyclic-heteroaryl are independently optionally substituted with one or two substituents selected from methyl, ethyl, fluorine, chlorine and methoxy.

8. The compound for use of any one of the claims 1 to 7, wherein said R₂ is selected from 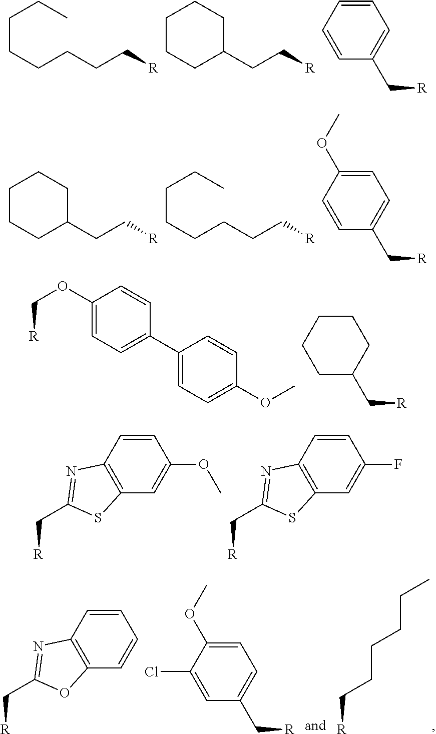
wherein R indicates the attachment to the CH-moiety depicted in formula (I).

9. The compound for use of any one of the claims 1 to 8, wherein said R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic or monocyclic heterocyclic ring, preferably a monocyclic carbocyclic ring, and wherein R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heteroaryl or a monocyclic heterocyclyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

10. The compound for use of any one of the claims 1 to 9, wherein said R₃ is selected from 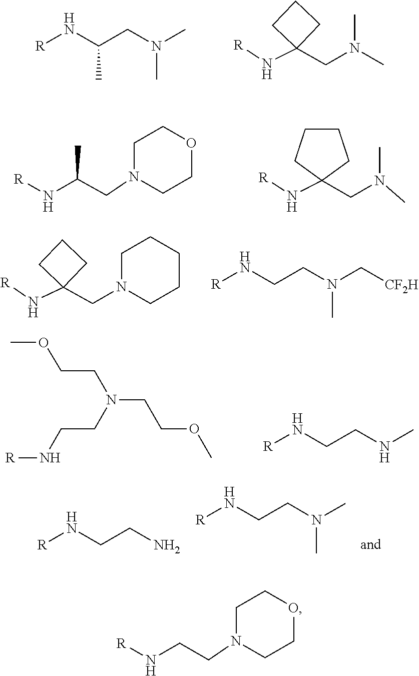
wherein R indicates the attachment to the C(O)-moiety depicted in formula (I).

11. The compound for use according to claim 1, wherein said compound is selected from

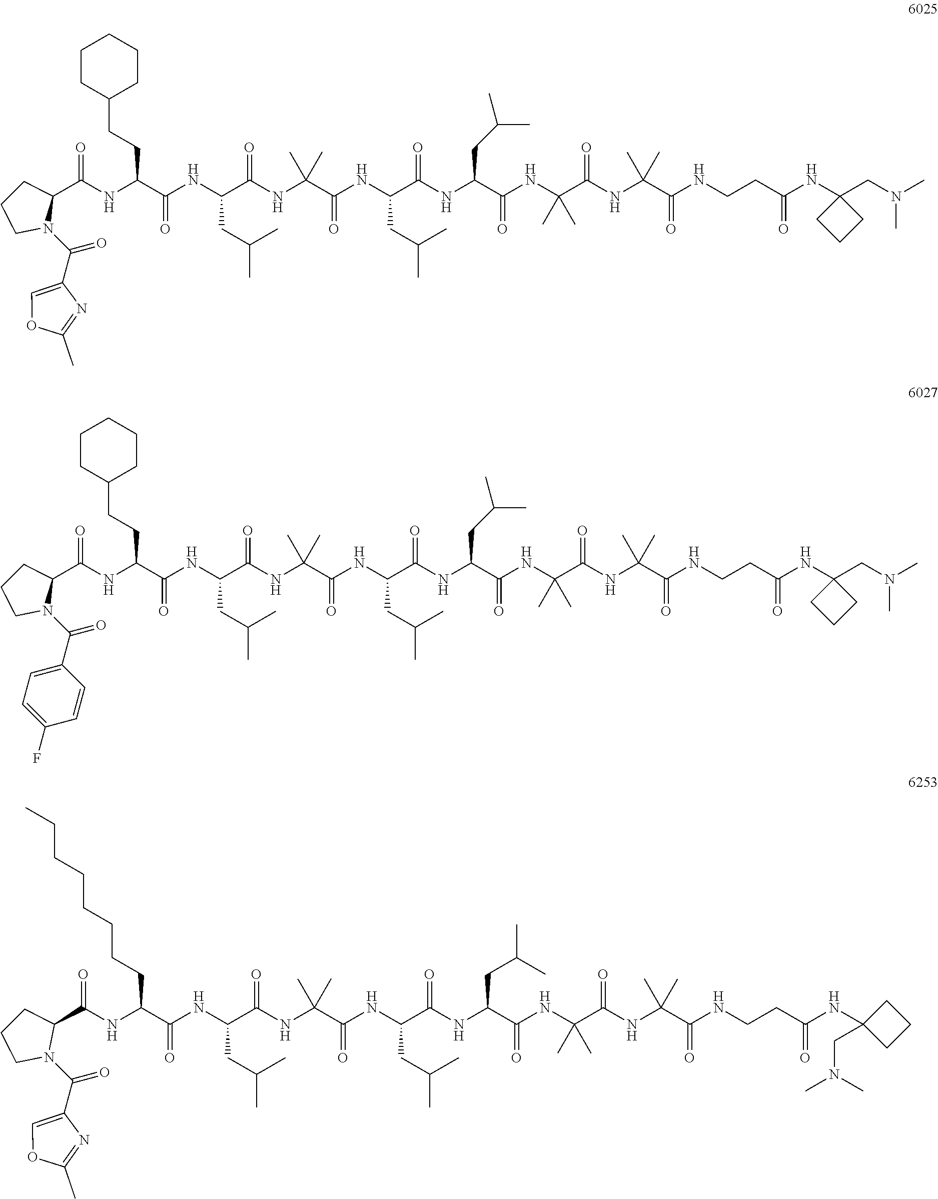

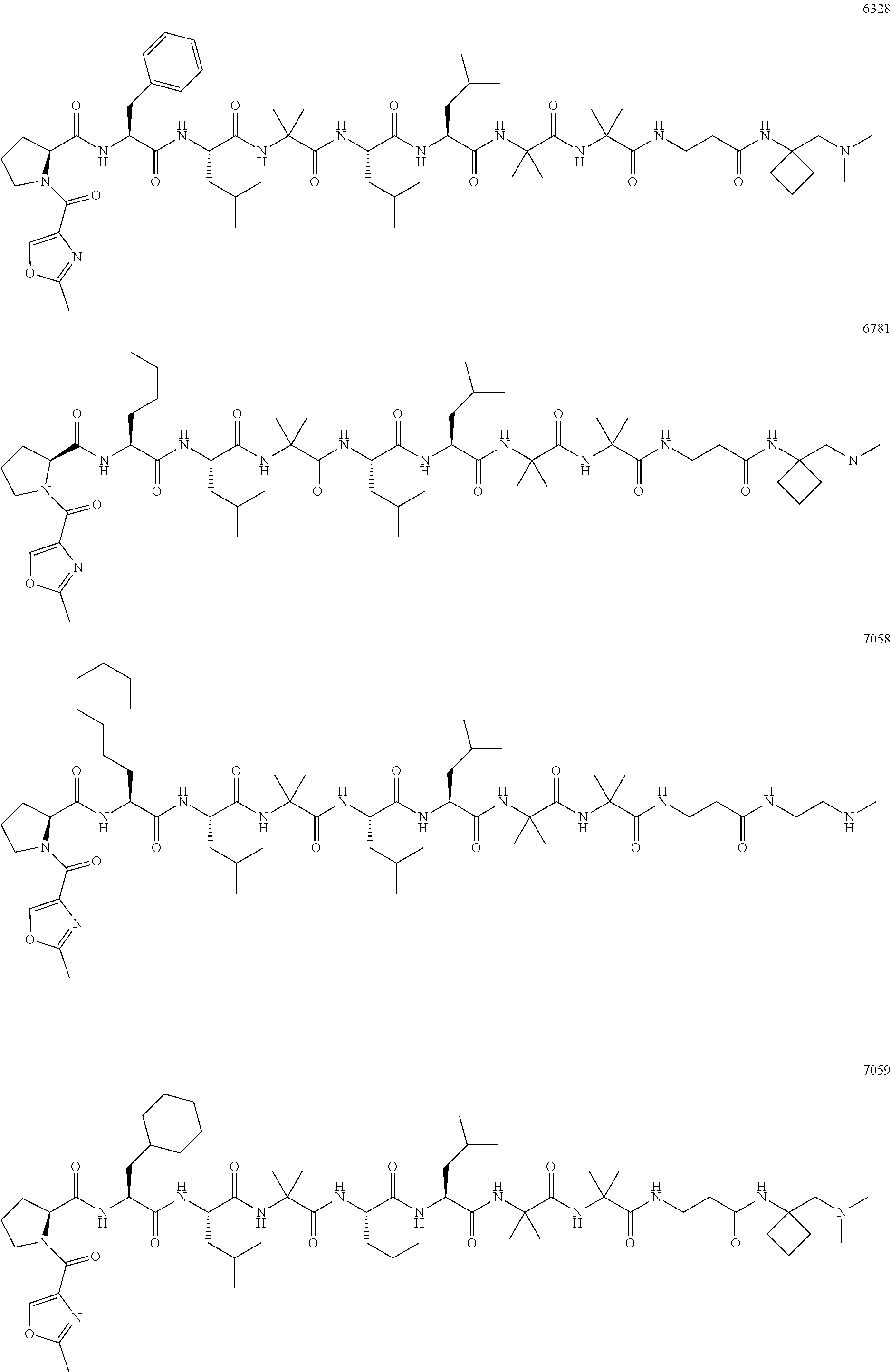

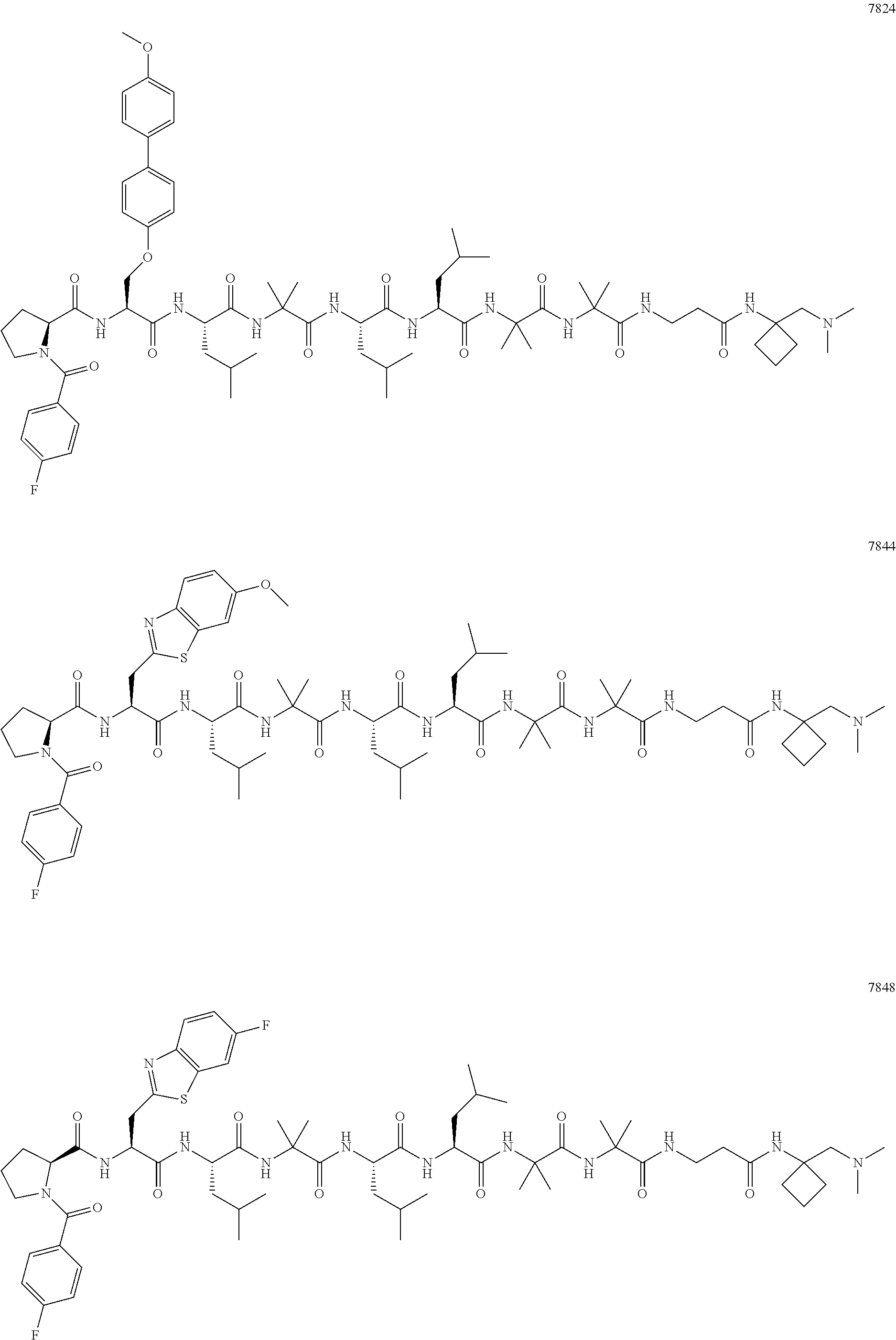

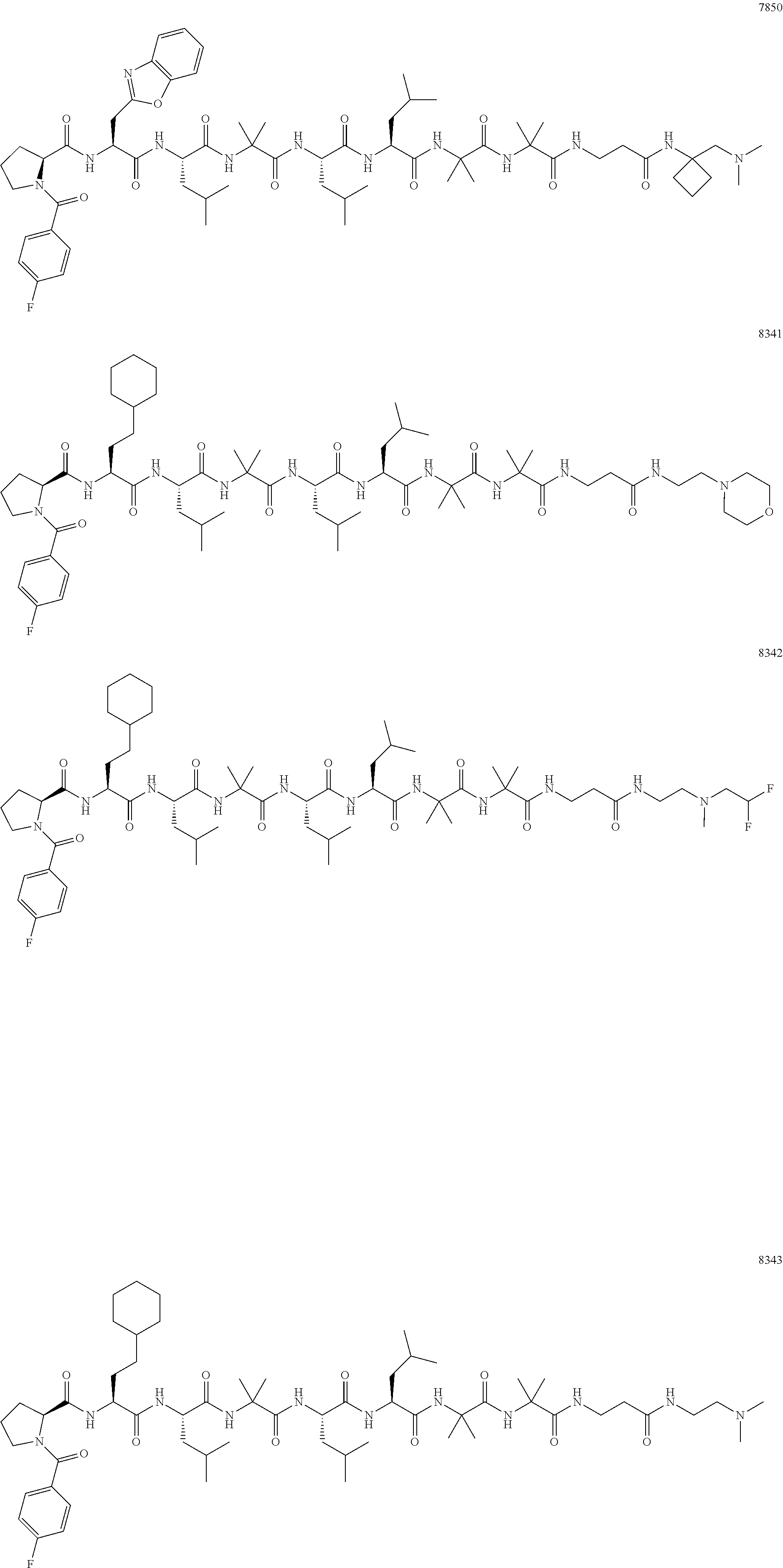

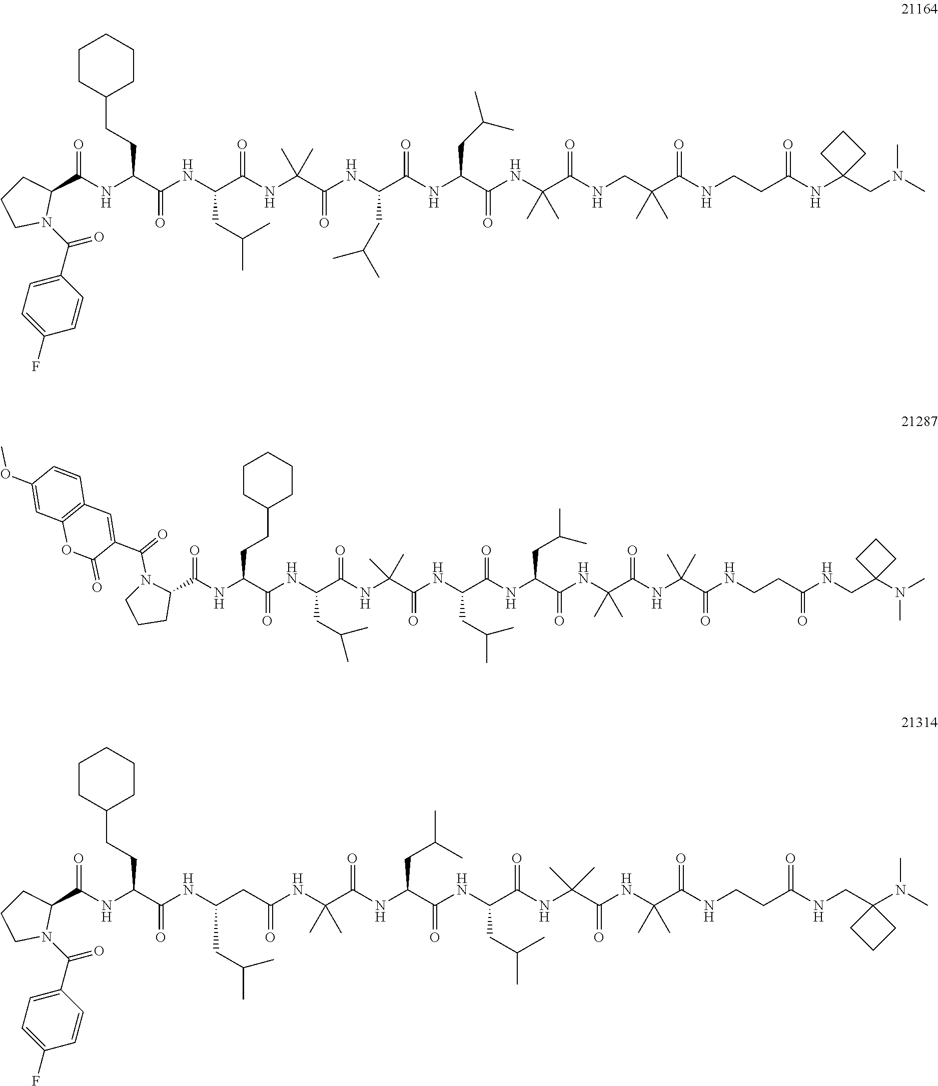

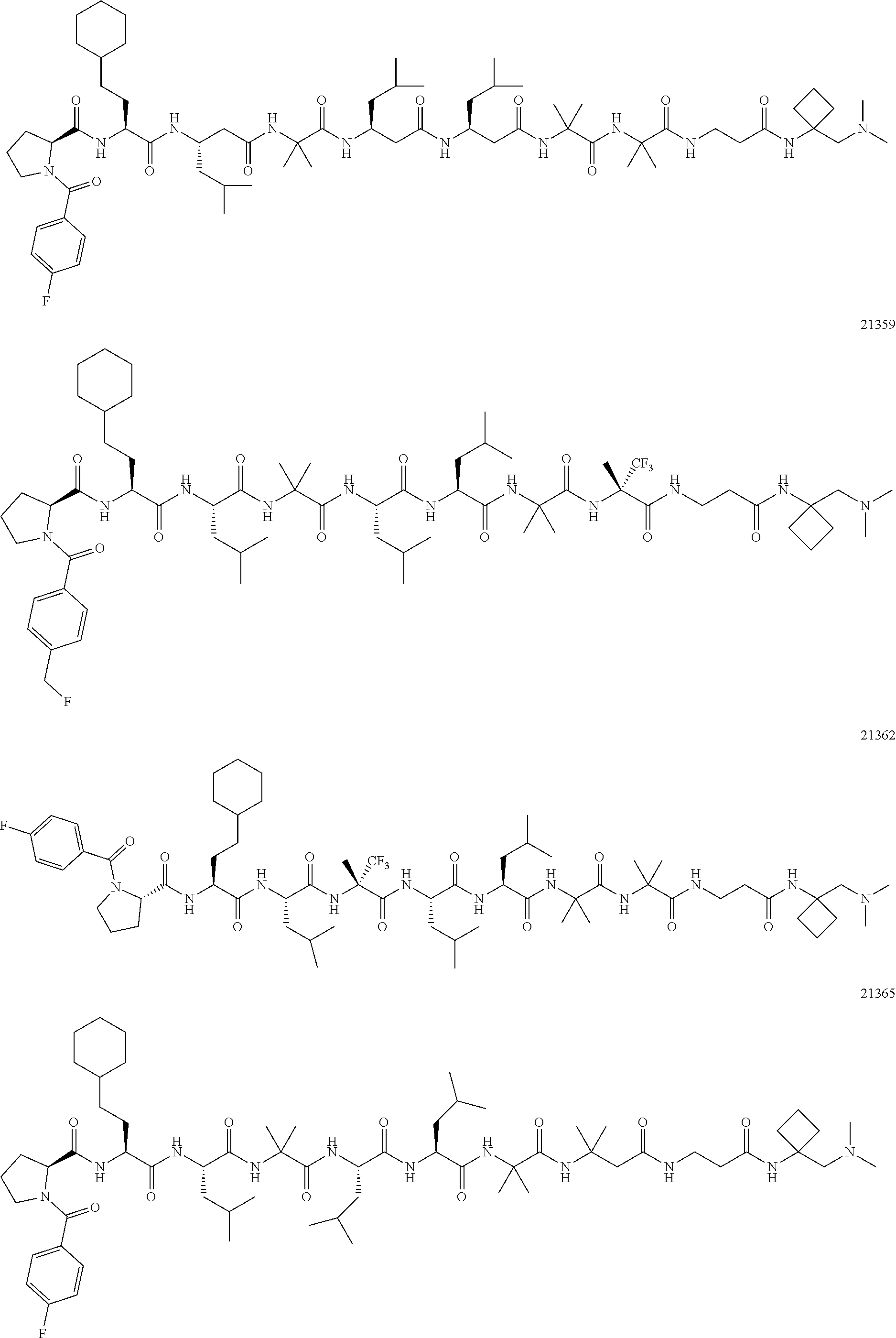

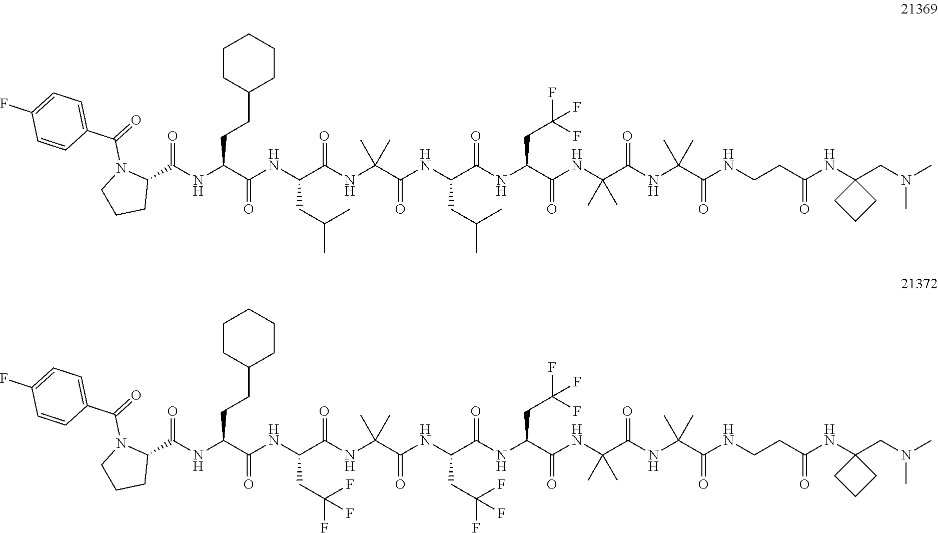 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(F)(F)F)C(=O)NC(C)(C)C(=O)NC(C)(C)C(=O)NCCC(=O)NC1(CN(C)C)CCC1 ; CN(C)CC1(NC(=O)CCNC(=O)C(C)(C)NC(=O)C(C)(C)NC(=O)[C@H](CC(F)(F)F)NC(=O)[C@H](CC(F)(F)F)NC(=O)C(C)(C)NC(=O)[C@H](CC(F)(F)F)NC(=O)[C@H](CCC2CCCCC2)NC(=O)[C@@H]2CCCN2C(=O)c2ccc(F)cc2)CCC1]

12. The compound for use of any one of the claims 1 to 11, wherein said topical administration is applying said compound to a skin of a mammal, preferably to a skin of a human.

13. The compound for use of any one of the claims 1 to 12, wherein said skin cancer is selected from a melanoma and non-melanoma skin cancer, preferably wherein said skin cancer is a melanoma.

14. The compound for use of any one of the claims 1 to 12, wherein said skin cancer is a non-melanoma skin cancer, wherein preferably wherein said non-melanoma skin cancer is selected from a basal cell carcinoma or a cutaneous squamous cell carcinoma.

15. A pharmaceutical composition for use in a method of topical treatment of a skin cancer of a mammal, wherein said pharmaceutical composition comprises a compound of formula (I) or a pharmaceutically acceptable salt thereof as defined in any one of the claims 1 to 14 and a pharmaceutically acceptable carrier or adjuvant, and wherein said method comprises topical administration of said pharmaceutical composition to said mammal.
